(19) United States
(12) Patent Application Publication
(10) Pub. No.: US 2005/0203549 A1
(43) Pub. Date: Sep. 15, 2005

(54) Methods and apparatus for off pump aortic valve replacement with a valve prosthesis

(76) Inventor: Fidel Realyvasquez, Palo Cedro, CA (US)

Correspondence Address: HELLER EHRMAN LLP, 275 MIDDLEFIELD ROAD, MENLO PARK, CA, 94025-3506, US

(21) Appl. No.: 11/077,192

(22) Filed: Mar. 9, 2005

Related U.S. Application Data
(60) Provisional application No. 60/551,992, filed on Mar. 9, 2004.

(51) Int. Cl. A61B 17/10

(52) U.S. Cl. 606/142; 623/2.11

(57) ABSTRACT

Methods and apparatus are provided for valve repair or replacement. In one embodiment, the method comprises providing an apparatus having a valve prosthesis, a valve leaflet support and a valve excisor, the apparatus having a first configuration and a second configuration; accessing the aortic root without placing the patient on a heart-lung machine; advancing the apparatus in the first configuration where the valve leaflet support is advanced through a valve, wherein the support is positioned below a valve annulus; expanding the apparatus into a second configuration so that the support will engage the valve; and moving the valve leaflet support and valve excisor together to remove leaflets of the valve. Penetrating members may be advanced into the tissue wherein the penetrating members may act as fasteners to hold the prosthesis in place.

### CROSS-REFERENCE TO RELATED APPLICATION

The present application claims the benefit of priority to copending U.S. Provisional Application No. 60/551,992 fully incorporated herein by reference for all purposes.

### BACKGROUND OF THE INVENTION

The invention relates to apparatus and methods for minimally invasive heart valve replacement and is especially useful in aortic valve repair procedures.

Essential to normal heart function are four heart valves, which allow blood to pass through the four chambers of the heart in one direction. The valves have either two or three cusps, flaps, or leaflets, which comprise fibrous tissue that attaches to the walls of the heart. The cusps open when the blood flow is flowing correctly and then close to form a tight seal to prevent backflow.

The four chambers are known as the right and left atria (upper chambers) and right and left ventricles (lower chambers). The four valves that control blood flow are known as the tricuspid, mitral, pulmonary, and aortic valves. In a normally functioning heart, the tricuspid valve allows one-way flow of deoxygenated blood from the right upper chamber (right atrium) to the right lower chamber (right ventricle). When the right ventricle contracts, the pulmonary valve allows one-way blood flow from the right ventricle to the pulmonary artery, which carries the deoxygenated blood to the lungs. The mitral valve, also a one-way valve, allows oxygenated blood, which has returned to the left upper chamber (left atrium), to flow to the left lower chamber (left ventricle). When the left ventricle contracts, the oxygenated blood is pumped through the aortic valve to the aorta.

Certain heart abnormalities result from heart valve defects, such as valvular insufficiency. Valve insufficiency is a common cardiac abnormality where the valve leaflets do not completely close. This allows regurgitation (i.e., backward leakage of blood at a heart valve). Such regurgitation requires the heart to work harder as it must pump both the regular volume of blood and the blood that has regurgitated. Obviously, if this insufficiency is not corrected, the added workload can eventually result in heart failure.

Another valve defect or disease, which typically occurs in the aortic valve is stenosis or calcification. This involves calcium buildup in the valve which impedes proper valve leaflet movement.

In the case of aortic valve insufficiency or stenosis, treatment typically involves removal of the leaflets and replacement with valve prosthesis. However, known procedures have involved generally complicated approaches that can result in the patent being on cardio-pulmonary bypass for an extended period of time.

Applicants believe that there remains a need for improved valvular repair apparatus and methods that use minimally invasive techniques and/or reduce time in surgery. Although known technology have described methods to replace a human aortic valve with a prosthesis, these methods are, however, designed to be used while the patient is on cardiopulmonary bypass and an open aorta technique. It is understood that there are potentially adverse effects from cardiopulmonary bypass. Recently, methods have been introduced to insert a stented aortic valve using percutaneous techniques but, unfortunately, the native aortic valve is left in situ and presently limited to very ill patients not suitable for valve replacement by conventional means. The need remains for further improved methods of valve repair and/or replacement.

### SUMMARY OF THE INVENTION

The present invention provides solutions for at least some of the drawbacks discussed above. Specifically, some embodiments of the present invention provide improved methods for treating various aortic valve ailments. In one embodiment, the present invention provides an alternative technique where the native aortic valve is replaced using a partial or full sternotomy while the patient is under general anesthesia but without cardiopulmonary bypass assistance. Advantageously, the patient may have a more rapid recovery and improved outcomes using such a minimally invasive cardiac surgery technique. In one embodiment, the present technique is intended to be used in patients who are not candidates for conventional aortic valve replacement techniques and would be suited for patients who need aortic valve replacement because of a severely regurgitant aortic valve with thin or fibrotic leaflets and minimal calcification. At least some of these and other objectives described herein will be met by embodiments of the present invention.

In one embodiment, the present invention provides a method of off-pump valve replacement. The method comprises the following: once the patient is under general anesthesia and a partial or full sternotomy is completed, the ascending aorta is exposed. The method then comprises accessing the aortic root; engaging or deploying the aortic cone anvil; deploying the aortic valve prosthesis; extracting the assembly; and closing the aortic access. In one embodiment, accessing the aortic root may involve attaching a graft to the aorta. The graft may include a hemostatic cap and may include a minimially invasive device for delivery of a valve prosthesis.

More specifically in one embodiment, the method comprises providing an apparatus having a valve prosthesis, a valve leaflet support and a valve excisor, the apparatus having a first configuration and a second configuration; accessing the aortic root without placing the patient on a heart-lung machine; advancing the apparatus in the first configuration where the valve leaflet support is advanced through a valve, wherein the support is positioned below a valve annulus; expanding the apparatus into a second configuration so that the support will engage the valve; and moving the valve leaflet support and valve excisor together to remove leaflets of the valve. Penetrating members may be advanced into the tissue wherein the penetrating members may act as fasteners to hold the prosthesis in place.

In one embodiment of the present invention, the method may involve accessing the aortic root without placing the patient on a heart-lung machine further comprises attaching a graft to the aorta. The aortic root may be accessed without placing the patient on a heart-lung machine and further comprises: attaching a first graft to the aorta, clamping said first graft, and attaching a second graft to the first graft, wherein the second graft includes a hemostatic cap on a proximal end to prevent excessive blood loss and the apparatus contained in the second graft, wherein the apparatus is movable out of the graft and into the aorta. The graft may be compressed in an accordian-like fashion when the apparatus is advanced. The method further comprise using a ratchet to move the valve support to engage it against the valve leaflets. The apparatus may further comprise using a ratchet to move the valve support to engage it against said valve leaflets, wherein the ratchet extends outward from the hemostatic cap. The method may include injecting the patient with medication to cause temporary asystole. The method may include injecting the patient with adenosine phosphate. A pericardial tent may be used on the apparatus to shield leaflets on the valve prosthesis from being damaged by the cutting element. A tent may also be used to capture leaflets and tissue that has been excised. The valve excisor may be turned with simultaneous proximal counter traction to remove the valve leaflets. The valve leaflets may be captured between the pericardial tent and the cutting element. The apparatus may be delivered minimally invasively. The attachment may comprise delivering a shape memory clip that holds the valve prosthesis to the tissue.

In one embodiment of the present invention, a valve delivery device comprises a heart valve prosthesis support having a proximal portion and a distal portion. The device may have a plurality of fasteners ejectably mounted on the support and a heart valve prosthesis that is releasably coupled to the distal portion of said heart valve prosthesis support. The heart valve prosthesis and support may be configured for delivery to the heart through an aortotomy formed in the patient's aorta. The device may include a valve excisor and an anvil movable along a longitudinal axis of the device to engage tissue disposed between the anvil and the valve prosthesis.

The device may include a pericardial tent positioned to capture valve leaflets between the tent and the valve excisor.

In another embodiment of the present invention, a valve delivery device comprises a heart valve prosthesis support having a proximal portion and a distal portion, a plurality of fasteners ejectably mounted on the support, and a heart valve prosthesis being releasably coupled to said distal portion of said heart valve prosthesis support. The heart valve prosthesis and support may be configured for delivery to the heart through an aortotomy formed in the patient's aorta. The device may include a valve cutting element and a pericardial tent positioned to capture valve leaflets between the tent and the valve cutting element. The device may also include a leaflet support movable along a longitudinal axis of the device to engage tissue disposed between the support and the valve prosthesis.

In a still further embodiment of the present invention, an end-to-side access device comprises a first portion of a tissue clamp; a second portion of the tissue clamp, wherein the second portion has a collapsed configuration and an expanded configuration. The device may also include a tissue cutter and a hollow shaft wherein the second portion may be delivered through said hollow shaft into the vessel to engage an inner surface of the wall of the vessel while the first portion engages an outer surface of the wall. The tissue cutter may be configured to cut the tissue engaged by the tissue clamp, said first portion and second portion slidable to remove tissue engaged between the tissue clamp from the vessel. The device may further include a graft housing said tissue cutter and tissue clamp. The device may also include a ring of fasteners used to secure the graft to the tissue. A hemostatic cap may be attached to on the graft.

In a still further embodiment of the present invention, a device is provided for use with a heart surgery device in an off-pump heart procedure. The device comprises a blood containment device wherein one portion of the device is configured to be attached to a blood vessel while blood is flowing in the vessel; a hemostatic cap located on another portion of the blood containment device, wherein the hemostatic cap allows a portion of the heart device to extend through the hemostatic cap without creating a leak; and a vent allowing for air to be removed from the containment device.

The blood containment device may be sized to house at least a portion of a valve prosthesis delivery device therein. A release device may allow the portion of the device with the hemostatic cap to be removed from the portion of the containment device attached to the blood vessel. The blood containment device may be a graft or a graft made of a pliable, flexible material that can be clamped to created a fluid seal. The blood containment device may be attached to the blood vessel without substantial blood leakage and without creating a life-threatening loss of blood. The blood containment device may house a portion of a multi-fire valve prosthesis delivery device. The blood containment device has a diameter of less than 30 mm, less than 20 mm, or less than 10 mm. The blood containment device may be sized to fit inside a four inch incision into the sternum.

In yet another embodiment of the present invention, a method is provided for valve replacement. The method comprises providing a valve prosthesis delivery apparatus; attaching a blood containment device to the aorta to prevent substantial loss of blood when the aorta is cut to provide access to an interior of the heart; cutting the aorta to access the aortic root without placing the patient on a heart-lung machine; advancing the apparatus to the area of the diseased valve; delivering the valve to the target site; retracing the valve prosthesis delivery apparatus into the blood containment device; and sealing off a portion of the containment device to allow the delivery device contained therein to be removed without significant blood loss.

For this method, the blood containment device may be sized to house at least a portion of a valve prosthesis delivery device therein. A release device may allow the portion of the device with the hemostatic cap to be removed from the portion of the containment device attached to the blood vessel. The blood containment device may be a graft or a graft made of a pliable, flexible material that can be clamped to created a fluid seal. The blood containment device may be attached to the blood vessel without substantial blood leakage and without creating a life-threatening loss of blood. The blood containment device may house a portion of a multi-fire valve prosthesis delivery device. The blood containment device has a diameter of less than 30 mm, less than 20 mm, or less than 10 mm. The blood containment device may be sized to fit inside a four inch incision into the sternum.

A further understanding of the nature and advantages of the invention will become apparent by reference to the remaining portions of the specification and drawings.

### BRIEF DESCRIPTION OF THE DRAWINGS

FIG. 1 illustrates an aortic root pulled back to show the aortic valve leaflets to be removed in an aortic valve replacement procedure of the present invention;

FIG. 2A is perspective view of minimally invasive valve cutting apparatus suitable for removing the valve leaflets from an aortic valve in accordance with the present invention and shown in a collapsed state;

FIG. 2B is a perspective view of the apparatus of FIG. 2A shown in an expanded state and illustrated for exemplary purposes positioned in an aortic valve;

FIG. 2C is a perspective view of the apparatus of FIG. 2B illustrating the cutting members of the apparatus engaged after cutting the aortic valve leaflets from the aortic valve;

FIG. 3A is a perspective view of another minimally invasive valve cutting apparatus in accordance with the present invention;

FIGS. 3B, 3C, and 3D are diagrammatic partial sectional views of the apparatus of FIG. 3A where FIG. 3B shows the pair of cooperating cutting elements of the apparatus above the valve leaflets, FIG. 3C shows one of the cooperating cutting elements positioned below the valve leaflets, and FIG. 3D shows the upper cooperating cutting element rotated and the valve leaflets separated form the original valve;

FIG. 4A is a perspective view of valve prosthesis and clip delivery apparatus in accordance with the invention shown supporting valve prosthesis and being in a collapsed state for minimally invasive delivery of the valve prosthesis (e.g., through an aortotomy);

FIG. 4B is another perspective view of the delivery apparatus of FIG. 4A with the support arm slide retracted to place the arms in an expanded state;

FIG. 4C is another perspective view of the delivery apparatus of FIG. 4A with clip ejection actuator moved distally to eject the fasteners, which fasten the valve prosthesis to the surgical site;

FIG. 4D is another perspective view of the delivery apparatus of FIG. 4A illustrating removal of the delivery apparatus after the clips have been released;

FIGS. 5A-5D are partial sectional views of the distal end of the delivery apparatus of FIG. 4A and the valve prosthesis seated on an aortic valve diagrammatically illustrating clip delivery where FIG. 5A shows the ends of the support arms penetrated through the sides of the replacement valve, FIG. 5B shows the ejection of the clips into the aortic root wall, FIG. 5C illustrates withdrawal of the ends of the support arms and the clips fully released and securing the valve prosthesis to the aortic valve annulus, and FIG. 5D illustrates complete removal of the prosthesis and clip delivery apparatus;

FIG. 5E is a detailed view illustrating a pusher member of the valve prosthesis and clip delivery apparatus ejecting a clip;

FIG. 5F illustrates the clip of FIG. 5E discharges from the delivery apparatus support arm and in place where it secures a portion of the valve prosthesis to the aortic annulus;

FIG. 6 illustrates how the valve prosthesis attachment would appear if the aortic root were cut and pulled back after implantation;

FIG. 7 illustrates placement of an expandable balloon within the valve prosthesis after the valve prosthesis is secured to the aortic annulus with the balloon expanded and compressing the outer wall surfaces of prosthesis having bio-glue applied thereto against the aortic inner wall;

FIG. 8 is a perspective view of the delivery apparatus of FIG. 4A supporting a mechanical valve;

FIG. 9A is a side view of the mechanical valve of FIG. 8 in an open state;

FIG. 9B is a side view of the mechanical valve of FIG. 8 in a closed state;

FIG. 10 is a perspective view of the mechanical valve secured to the aortic annulus after delivery with the delivery apparatus of FIG. 9; and

FIG. 11 is a top plan view the fastener clip depicted in various of the foregoing Figures shown in a relaxed or free state.

FIG. 12 shows a prosthesis delivery device for use with a support device.

FIG. 13 shows one embodiment of the support device.

FIG. 14 shows a cross-sectional view of the aorta with a graft according to the present invention.

FIGS. 15A to 15C shows embodiments of the present invention attached to or inside the aorta.

FIGS. 16A to 17 shows embodiments of the present invention attached to or inside the aorta.

FIG. 18 shows the device of the present invention removed from the aorta.

FIG. 19 shows a valve prosthesis in position.

FIGS. 20 through 26B show various embodiments of a valve leaflet cutter according to the present invention.

FIGS. 27A and 27B show a graft with a valve delivery device therein.

FIG. 28A through 38 show various embodiments of devices for removing a section of tissue from a tubular vessel.

FIG. 39 shows a view of an incision used to provide minimally invasive access.

FIG. 40 shows a perspective view of an aorta with a graft according to the present invention.

FIGS. 41A through 42B show various views of a graft attachment device.

FIGS. 43 through 46 show various views of a tissue cutter for use with a graft.

FIGS. 47 through 48B show various view of the cutter according to the present invention.

FIG. 49 through 52 show various views of a valve delivery device according to the present invention.

FIG. 53 shows one embodiment of a valve positioned in the aorta.

FIGS. 54A and 54B shows another embodiment of a valve for use with the present invention.

FIGS. 55A and 55B show various views of another prosthesis delivery device according to the present invention.

FIG. 56A shows one embodiment of a support device according to the present invention.

FIG. 56B shows one embodiment of a fastener housing according to the present invention.

FIGS. 57A-57B show various views of the device of FIG. 55B.

FIG. 58 shows a cross-sectional view of yet another embodiment of a delivery device according to the present invention.

FIG. 59A shows a valve prosthesis without a sewing ring.

FIG. 59B shows an enlarged cross-sectional view of the device.

FIG. 60A shows a portion of one embodiment of the hollow sharpened member.

FIG. 60B shows a cross-section of one embodiment of a fastener housing.

FIGS. 61 and 62 show enlarged cross-sectional views of a fastener being delivered to a secure a prosthesis.

### DESCRIPTION OF THE SPECIFIC EMBODIMENTS

It is to be understood that both the foregoing general description and the following detailed description are exemplary and explanatory only and are not restrictive of the invention, as claimed. It may be noted that, as used in the specification and the appended claims, the singular forms “a”, “an” and “the” include plural referents unless the context clearly dictates otherwise. Thus, for example, reference to “a material” may include mixtures of materials, reference to “a chamber” may include multiple chambers, and the like. References cited herein are hereby incorporated by reference in their entirety, except to the extent that they conflict with teachings explicitly set forth in this specification.

In this specification and in the claims which follow, reference will be made to a number of terms which shall be defined to have the following meanings:

“Optional” or “optionally” means that the subsequently described circumstance may or may not occur, so that the description includes instances where the circumstance occurs and instances where it does not. For example, if a device optionally contains a feature for using an inflatable valve support, this means that the inflatable feature may or may not be present, and, thus, the description includes structures wherein a device possesses the inflatable feature and structures wherein the inflatable feature is not present.

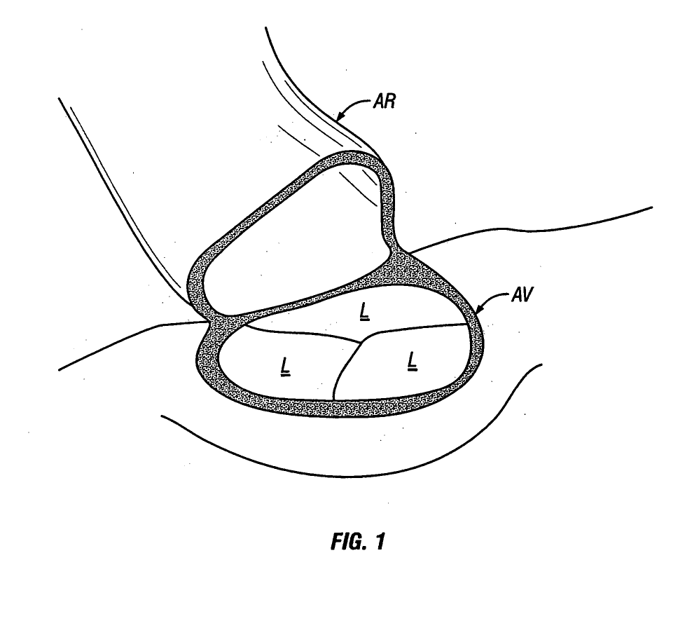

Referring to FIG. 1, an aortic root (AR) is shown pulled back to show the right, left, and posterior leaflets (L) of an aortic valve (AV) to be removed in a minimally invasive valve replacement procedure of the present invention where valve leaflet removal and valve prosthesis delivery apparatus can be delivered to the aortic root via an aortotomy.

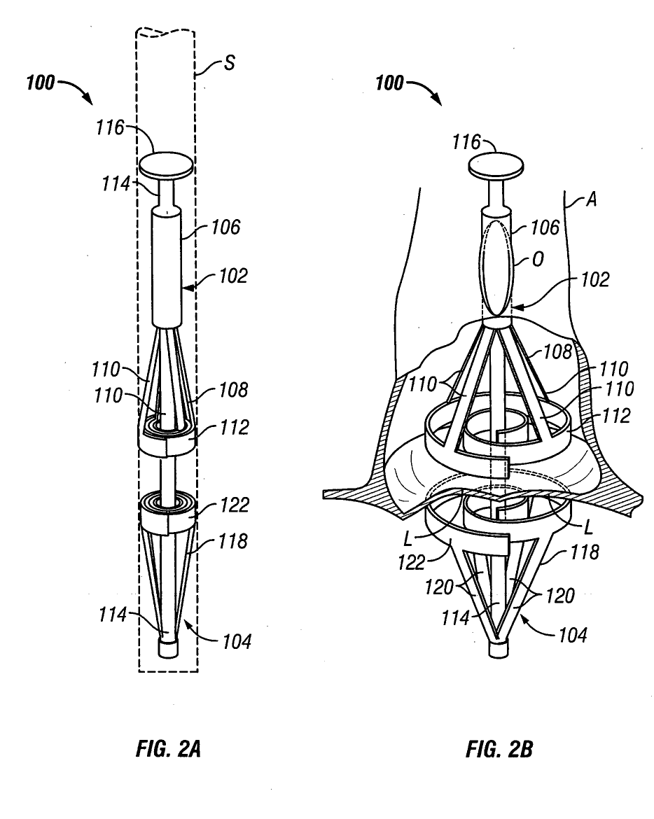

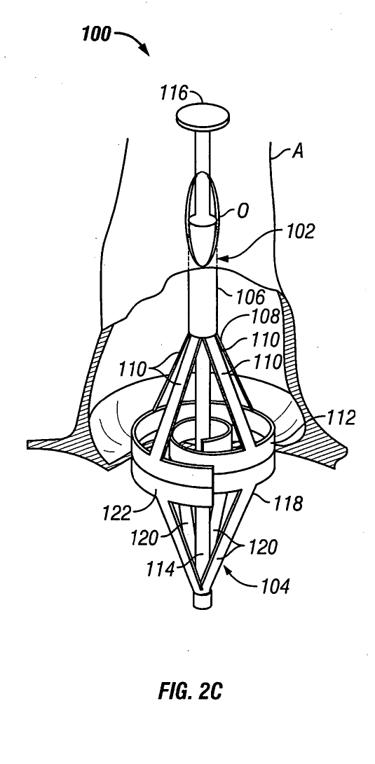

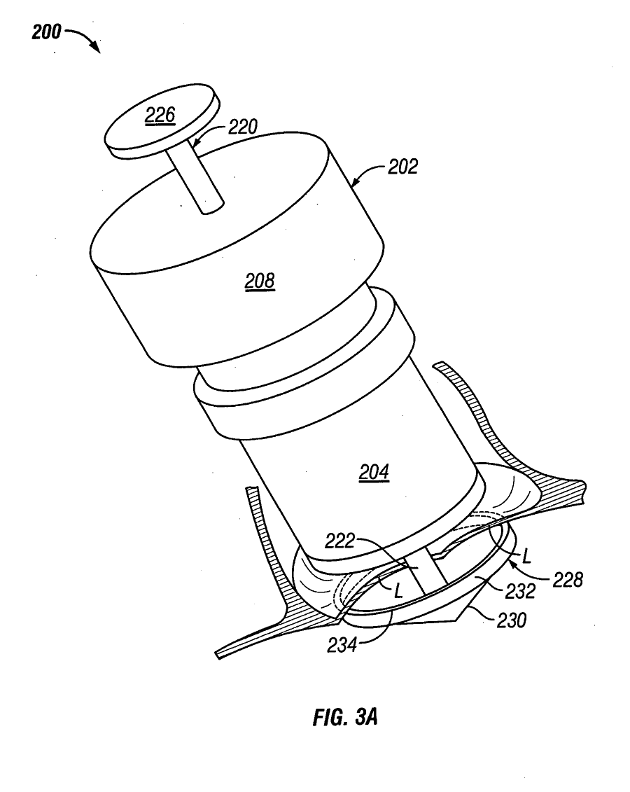

Referring to FIGS. 2A-C, one embodiment of minimally invasive valve cutting or removal apparatus is shown and generally designated with reference numeral 100. Apparatus 100 includes a first body member 102 and a second body member 104. First body member 102 includes a tubular member 106 and an umbrella having umbrella arms 110 and a cutting element 112, which is in the form of a spiral. Cutting element 112 can be formed from flat metal wire, such as flat stainless steel wire or ribbon or any other materials suitable cutting. Umbrella arms 110 each have one end secured to or integrally formed with tubular member 106 and one end secured to or integrally formed with cutting element 112.

Second body member 104 includes and elongated member 114, which can included a knob 116 at one end thereof. Second body member 104 also includes an umbrella 118, which is similar to umbrella 108. Umbrella 118 includes umbrella arms 120 and umbrella cutting element 122, which also is in the form of a spiral. Cutting element 122 can be formed from flat metal wire, such as flat stainless steel wire or ribbon or any other material suitable for cutting. Umbrella arms 120 each have one end secured to or integrally formed with elongated member 114 and one end secured to or integrally formed with cutting element 122.

As shown in FIG. 2A, the first and second umbrellas 108 and 118 are radially compressible or collapsible. A tube or sheath such as shown in dashed lines and indicated with reference character “S” in FIG. 2A can be placed around apparatus 100 to hold it in a collapsed state. With the sheath in place so that the umbrellas are in the radially compressed or collapsed state, where the umbrellas have a radial dimension less that of their uncompressed or uncollapsed state as shown in FIGS. 2A and 2B, sheath S and valve removal apparatus 100 are introduced through an opening 0 or aortotomy formed in the aorta (A) of a patient. When the second umbrella is positioned below the aortic leaflets (L) and the first umbrella is positioned above the aortic leaflets (L), the umbrellas are allowed to expand to their memory or relaxed state shown in FIG. 2B by retracting the sheath. If the umbrellas are not aligned as shown in FIG. 2A, members 106 and 114 can be manipulated to adjust the umbrella positions. Other mechanisms for holding elements 112 and 122 or the umbrellas radially compressed can be used. For example, a wire can be wrapped around elements 112 and 122 and pulled away from the apparatus when the umbrellas are in place and ready to deploy.

Referring to FIG. 2C, tubular member 106 and elongated member 114 are then moved in opposite directions to compress the leaflets between the opposed cutting edges of cutting elements 112 and 122, which edges can be sharpened to enhance cutting. Tubular member 106 and/or elongated member 114 also can be rotated to complete the cut if necessary. The cut leaflets can fall into second umbrella 118, which forms a holder for the leaflets if they do not remain between the cutting edges during removal of the apparatus.

Before removing the apparatus 100, it again is radially compressed. This can be done by sliding sheath S through over apparatus 100. If the second umbrella does not close with the first umbrella, the surgeon retract the apparatus so that the second umbrella is in the vicinity of the aortotomy and manipulate spiral cutting element 122 to reduce the diameter of the second umbrella. In this manner, apparatus 100, together with the cut leaflets are removed from the site through the aortotomy.

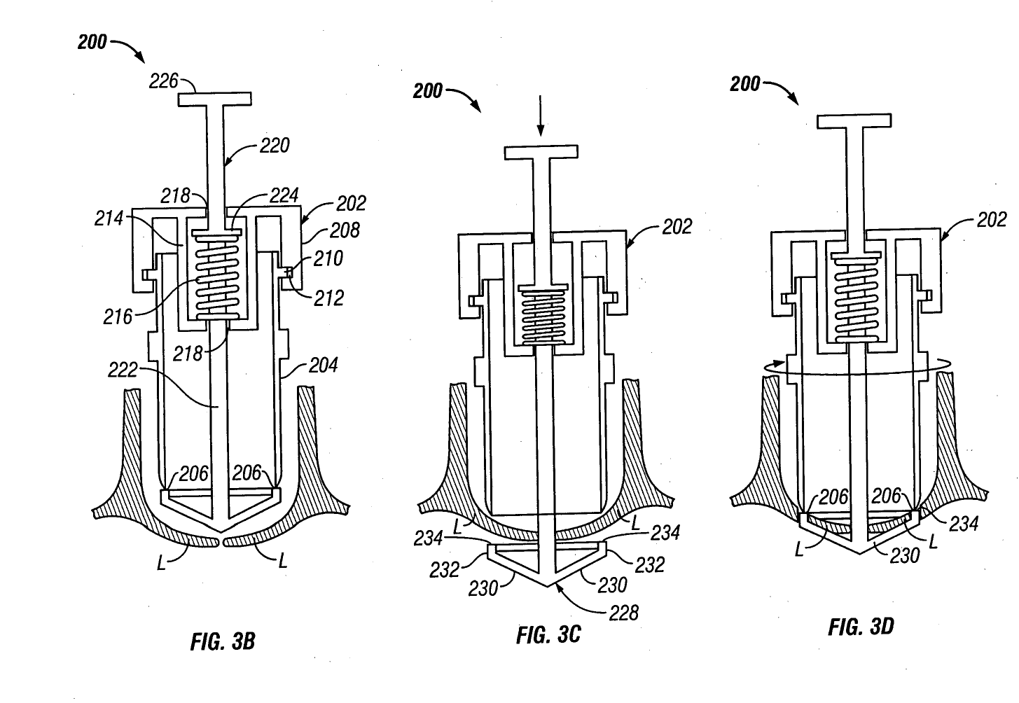

Referring to FIGS. 3A-D, another minimally invasive valve cutting or removal apparatus in shown accordance with the present invention and generally designated with reference numeral 200. Valve removal apparatus 200 generally includes a housing 202 and plunger 220 slidably mounted therein.

Housing 202 includes a first tubular portion or member 204, which has an annular cutting edge or element 206 at the distal end thereof, and a second portion or member 208 coupled thereto or integrally formed with first portion or member 204. First and second portions or members 204 and 206 can be rotatably coupled to one another through an annular tongue 210 and groove 212 arrangement as shown in FIGS. 3B-D. However, other coupling arrangements can be used and members 204 and 206 can be fixedly secured to one another. Second member or portion 208 includes a chamber 214 that houses and supports spring 216 and includes vertically aligned holes 218 through which plunger 220 is slidably mounted.

Plunger 220 includes an elongated member or rod 222 having an enlarged disc shaped portion 224 for interfacing with spring 216, a handle or knob 226 and a cutting and leaflet holding member 228 that cooperates with cutting edge 206. In the illustrative embodiment, cutting member 228 includes conical section 230 and cylindrical section 232, which forms annular cutting block or surface 234. Annular surface or element 234 cooperates with annular cutting edge or element 206 to cut the valve leaflets.

The distal portion of leaflet removal apparatus 200, which is adapted for passage through an aortotomy, is passed through such an aortotomy and positioned above the aortic valve leaflets a shown in FIG. 3B. Referring to FIG. 3C, the plunger is pressed or translated to position plunger cutting block 234 below the aortic leaflets. Compression spring 216 is allowed to return toward its relaxed state to drive the plunger proximally and squeeze the leaflets between surface 234 and cutting edge 206. In this position, housing portion 204 is rotated, as indicated with the arrow in FIG. 3D, to cut the leaflets. The cut leaflets fall into conical section or holder 230, which holds the cut leaflets as apparatus 200 is removed from the aortotomy.

According to another aspect of the invention, valve prosthesis delivery apparatus is provided to rapidly deliver the valve prosthesis to the surgical site and to secure the prosthesis at the desired location.

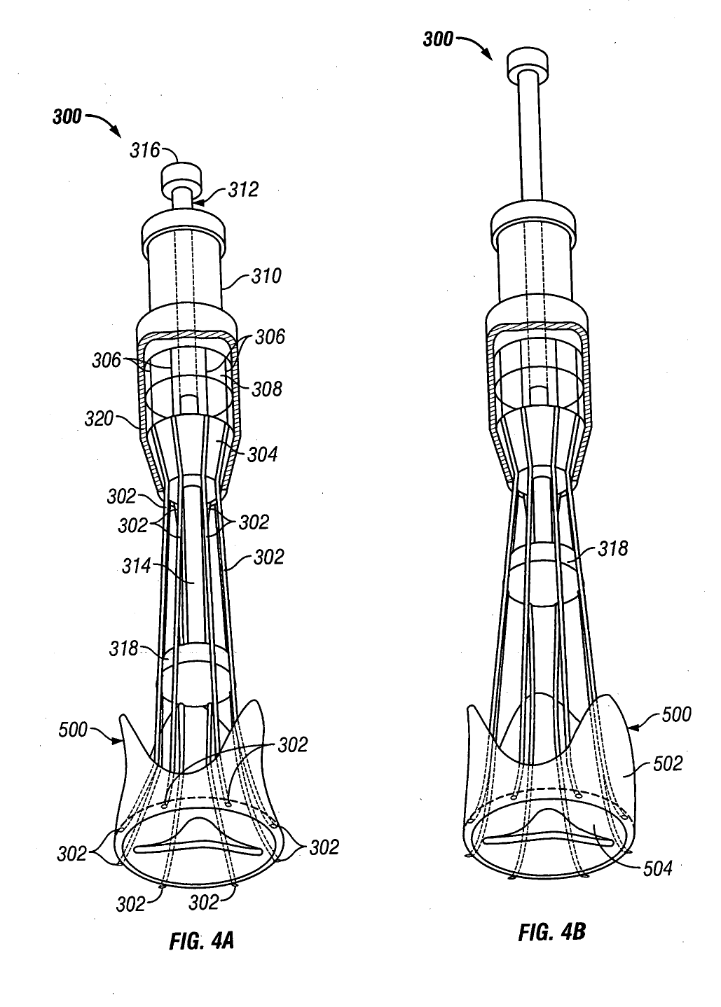

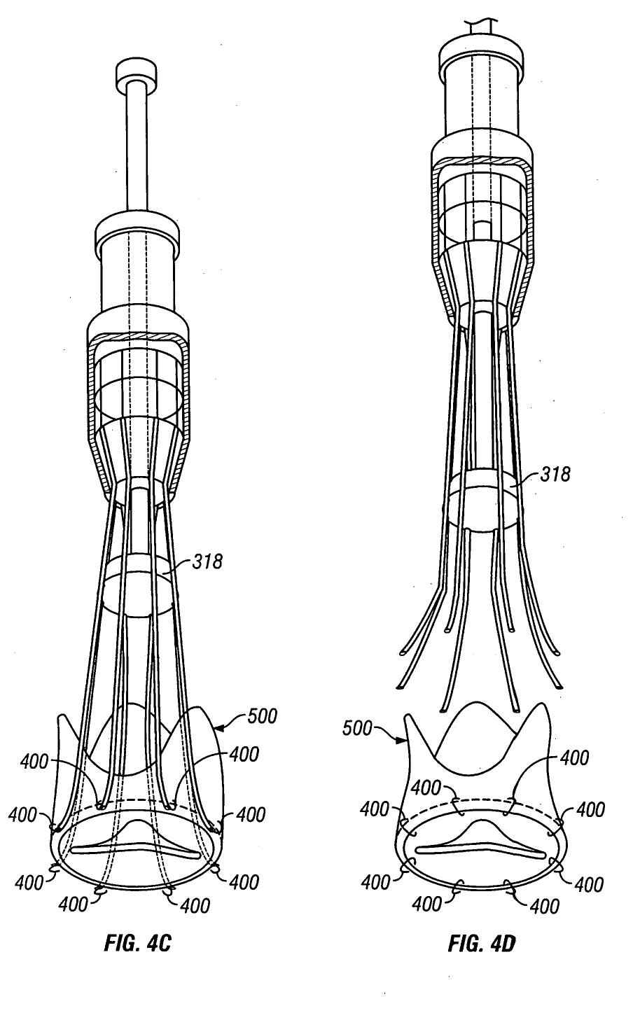

Referring to FIGS. 4A-C, an exemplary embodiment of a valve prosthesis delivery mechanism, which is generally designated with reference numeral 300, is shown. Valve prosthesis delivery apparatus 300 generally includes a support for supporting the prosthesis and a plurality of fastener ejectably mounted in the support.

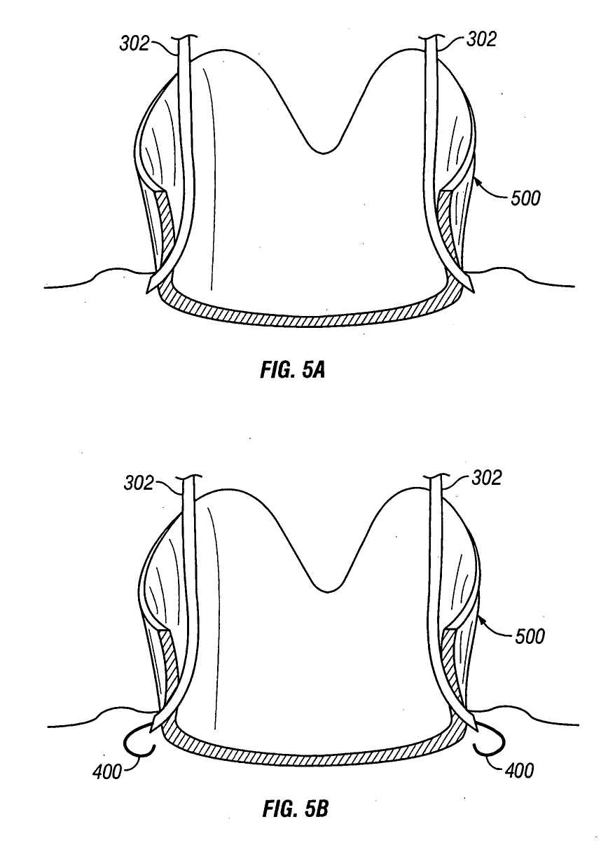

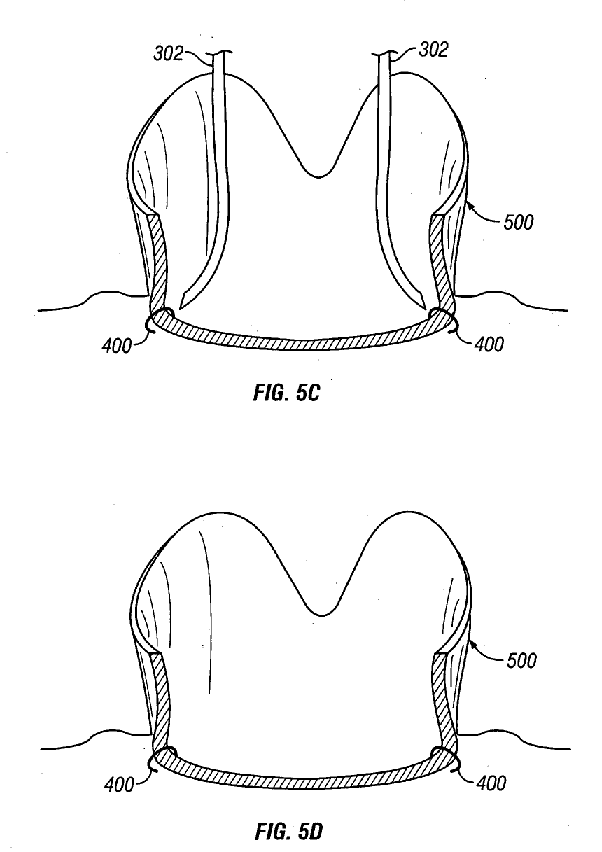

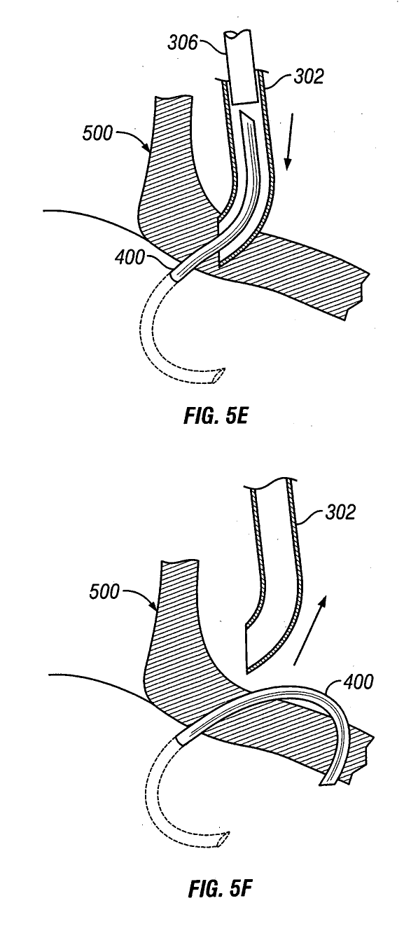

Referring to FIG. 4A, valve prosthesis mechanism 300 includes a prosthesis support comprising a plurality of tubes 302, each having a free distal end and a proximal portion fixedly secured to member 304, which in the illustrative embodiment, is frustoconical. A wire or pusher 306 is slidably mounted in each support tube 302 and includes a proximal portion that extends therefrom and is fixedly secured to plug 308, which can have the disc shape shown in the drawings. Grooves can be formed in member 304 and plug 308 for receiving support tubes 302 and wires 306, which can be formed form metal such as stainless steel, which has desirable stiffness. However, other suitable materials including nitinol can be used. Tubes 302 and wires 306 can be secured in the grooves by compressing sizing the grooves to be slightly smaller than the tubes and/or wires and/or by gluing. Plug 308 can be secured to cylindrical member 310 or integrally formed therein and form a portion thereof. Accordingly, when cylindrical member 310 is moved distally, wires 306 move distally to eject fastener clips 400 from support tubes 302 as shown in FIGS. 5E and 5F.

Valve prosthesis delivery apparatus 300 also can include apparatus or a mechanism for expanding support tubes 302 radially outward. In the illustrative embodiment, apparatus 300 includes a plunger 312, which includes elongated member 314. Elongated member 314 has a knob 316 at its proximal end and a slide member 318 at its distal end. Slide member 318 has a plurality of grooves formed therein in which support tubes 302 are slidably mounted. Slide member 318 is sized and/or configured so that when plunger 312 is moved proximally with slide member 318, slide member 318 urges support tubes radially outward. Plug 308 can be slidably mounted in a tubular housing 320, which can be secured to frustoconical member 304 as shown in the drawings. Housing 320 also is configured to slidably receive cylinder 310.

In use, valve prosthesis such as valve prosthesis 500 is secured to valve prosthesis delivery apparatus 300. Valve prosthesis 500 is shown as a conventional stentless tissue valve, which can be harvested from a suitable animal heart such as a porcine heart and prepared according to known methods. Valve prosthesis 500 includes a root portion 502 and a valve leaflet portion 504, which is shown in the drawings in an open position. In a closed configuration, the valve leaflet edges coapt to seal the valve and prevent regurgitation.

When securing valve prosthesis 500 to delivery apparatus 300, sliding member 318 is moved distally to allow the support tubes to return to their radially inward biased position as shown in FIG. 4A. Valve prosthesis 500 is then mounted on apparatus 300 so that a sharp pointed distal end of each support tube 302 extends through the lower wall portion of tissue valve prosthesis 500.

Referring to FIGS. 4A-D, FIG. 4A, sliding member 318 can be advanced to allow the support arms to move radially inward to a collapsed state as a result of the biasing effect of frustoconically shaped member 304. This position is used to introduce the apparatus through an aortotomy to the surgical site. FIG. 4B shows sliding member 318 retracted to place the arms in a radially expanded state. FIG. 4C shows cylinder 310 moved distally to eject the fastener clips 400, which are self-closing clips and fasten the valve prosthesis to the heart. FIG. 4D illustrates removal of the delivery apparatus after the clips have been released.

Self-closing clips 400 can comprise wire made from shape memory alloy or elastic material or wire so that it tends to return to its memory shape after being released from the clip delivery apparatus. As is well known in the art, shape memory material has thermal or stress relieved properties that enable it to return to a memory shape. For example, when stress is applied to shape memory alloy material causing at least a portion of the material to be in its martensitic form, it will retain its new shape until the stress is relieved as described in U.S. Pat. No. 6,514,265 to Ho et al. and which is hereby incorporated herein by reference. Then it returns to its original, memory shape. Accordingly, at least a portion of the shape memory alloy of clip 400 is converted from its austenitic phase to its martensitic phase when the wire is in its deformed, open configuration inside the curved distal end portion of a respective tube 302 (see e.g., FIG. 5E). When the stress is removed and clip 400 unrestrained, the material undergoes a martensitic to austenitic conversion and springs back to its undeformed configuration (FIG. 11).

One suitable shape memory material for the clip 400 is a nickel titanium (nitinol) alloy, which exhibits such pseudoelastic (superelastic) behavior.

The clip can be made by wrapping a nitinol wire having a diameter in the range of about 0.003 to 0.015 inch, and preferably 0.010 inch, and wrapping it around a mandrel having a diameter in the range of about 0.020 to 0.150, and preferably 0.080 inch. The heat treatment of the nitinol wire to permanently set its shape as shown in FIG. 11 can be achieved by heat-treating the wire and mandrel in either a convection oven or bath at a temperature range of 400 to 650° C., preferably 520° C., for a duration of 1 to 45 minutes, and preferably 15 minutes.

The following example is set forth with reference to FIGS. 5A-5E, 6, and 7 to further illustrate operation of valve prosthesis delivery apparatus 300 in replacing a malfunctioning aortic valve. It should be understood, however, that this example is not intended to limit its scope of the invention.

A patient is placed on cardiopulmonary bypass and prepared for open chest/open heart surgery, which typically requires a sternotomy. The surgeon removes the aortic leaflets using valve removal apparatus 100 or 200 as described above. Once the valve has been excised and removed with the valve removal apparatus, the surgeon then places a conventional aortic gazer through the aortotomy to determine the size of the aortic valve placement (e.g., valve prosthesis 500) as is known in the art.

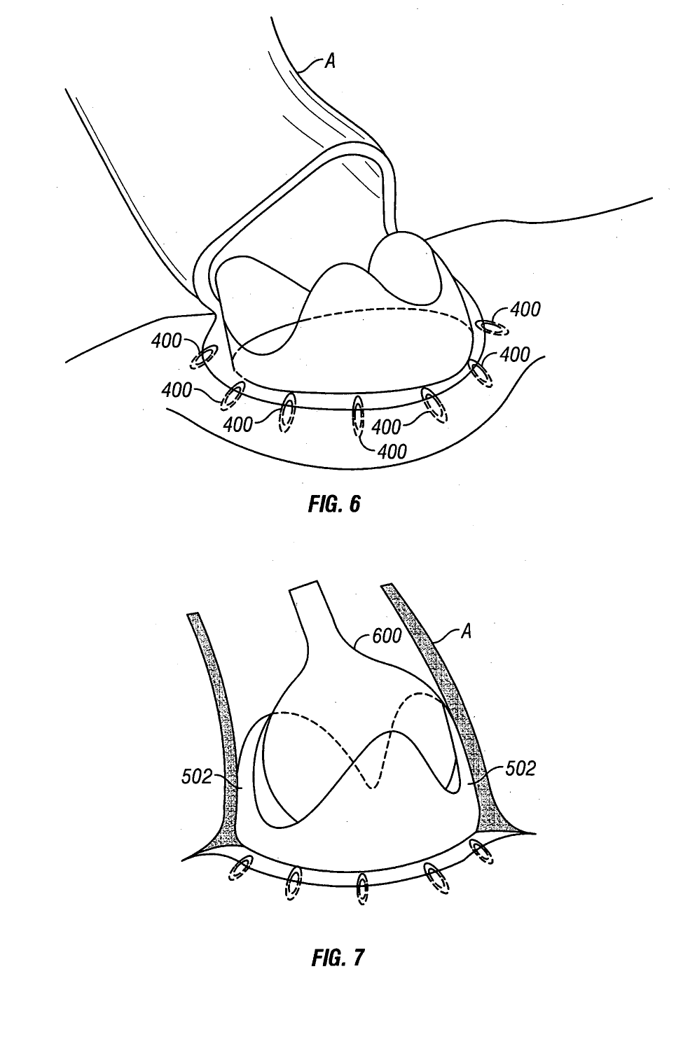

While in the generally collapsed state shown in FIG. 4A, valve prosthesis apparatus 300 is introduced through the aortotomy and the valve aligned with its natural location just below the two coronary arteries as is known in valve surgery. The sliding member 318 is retracted to have the piercing ends of support tubes 302 penetrate into the aortic root tissue as shown in FIG. 5A where the aorta is not shown for purposes of simplification. With valve prosthesis 500 seated and the sharp distal ends of the support arms 302 penetrated through the sides of the replacement valve 500 and slightly pushed further into adjacent the wall tissue, clips 400 are ejected into the adjacent wall tissue as shown in FIG. 5B. Specifically, cylinder 310 is moved distally so that pushers or wires 306 eject all of the clips 400 simultaneously (see FIGS. 4C and 5E). This one shot clip deliver can significantly reduce the time required to implant valve prosthesis as compared to other known techniques. After the clips are fully released and have tended to move toward their memory shape to secure valve prosthesis 500 in place as diagrammatically shown in FIG. 5C and more particularly in FIG. 5F, valve prosthesis delivery apparatus 300 is removed leaving the replacement valve secured at the desired site (FIG. 5D). FIG. 6 illustrates how the valve prosthesis attachment would appear if the aortic root were cut and pulled back after implantation.

Referring to FIG. 7, a conventional aortic balloon catheter including a balloon, such as balloon 600, is used to urging the outer surface of the root of the valve prosthesis against the inner wall of the aorta. Before introducing the valve prosthesis through the aortotomy, the outer surface of the root of the valve prosthesis is coated with bio-glue. Accordingly, as the balloon is expanded, it compresses the outer wall surfaces of prosthesis aortic root and the bio-glue applied thereto against the aortic inner wall and can hold it there while the glue sets. After the glue sets, the balloon is deflated and removed from the aortotomy and the aortotomy closed by conventional means.

Although the foregoing method has been described in connection with open chest surgery, the leaflet removal apparatus and prosthesis delivery apparatus described herein can be used with minimally invasive approaches that typically require a thoracotomy between adjacent ribs. Further, although the minimally invasive valve prosthesis replacement procedure has been described with reference to one prosthetic tissue valve, it should be understood that variations of such prosthesis or other valve prosthesis types can be used.

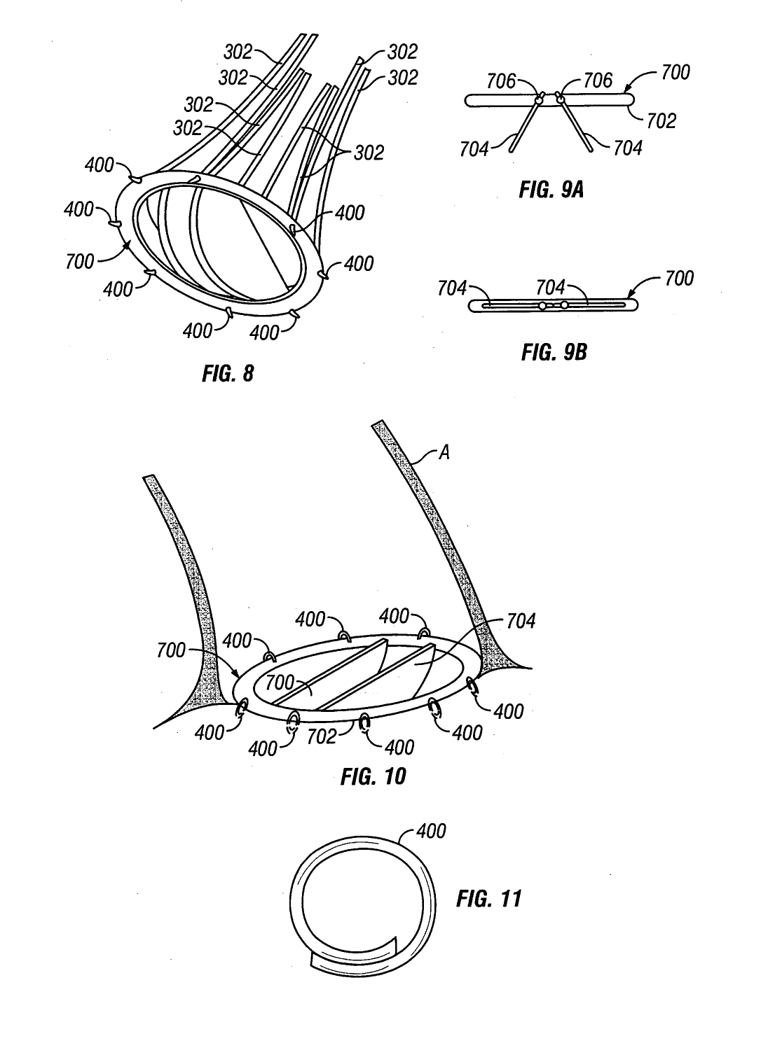

Referring to FIG. 8, valve prosthesis delivery apparatus 300 is shown in combination with a conventional mechanical hart valve prosthesis generally designated with reference numeral 700. Mechanical heart valve prosthesis 700 comprises an annular ring or housing 702, which can be metal or carbon material, to which two valve leaflets 704 are pivotally mounted. Each leaflet is pivotally mounted to ring 702 with two pivots 706 (two of the four pivots being hidden from view in FIG. 9A). A portion of each leaflet extends beyond its respective pivot as shown In FIG. 9A so that the leaflets can fully close the valve opening that ring 702 forms. Although a particular mechanical heart valve prosthesis is shown, it should be understood that any suitable mechanical heart valve prosthesis (or other valve prosthesis) can be used without departing from the scope of the invention. For example, a mechanical valve having a ball can be used.

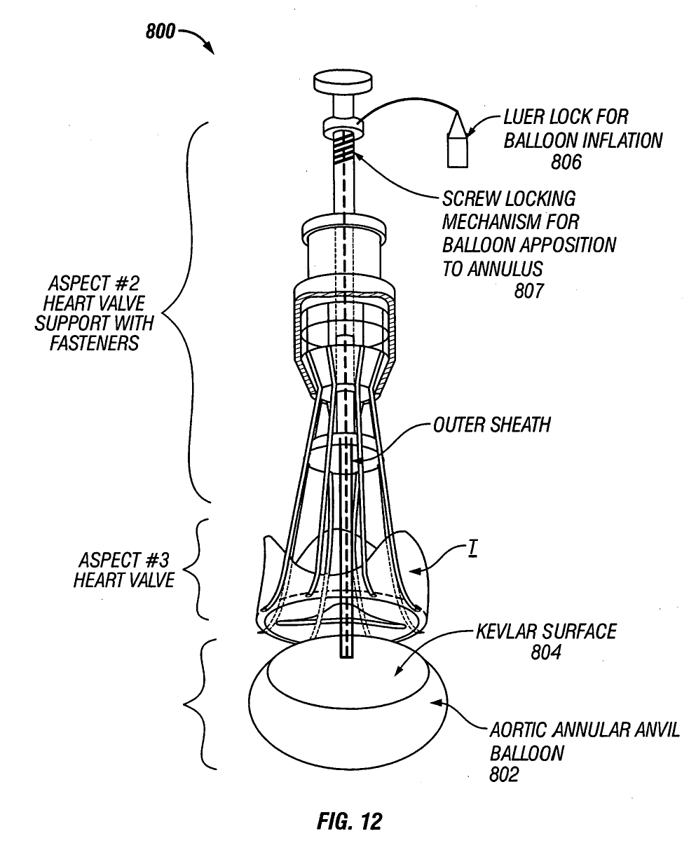

Referring now to FIG. 12, a still further embodiment of the present invention is shown. In this embodiment, an apparatus 800 is shown with an aortic anvil balloon 802. This balloon 802 is used to engage and/or grasp tissue T while clips and fasteners are being advanced by the apparatus 800. The balloon 802 may optionally be, but is not necessarily, integrated with the apparatus 800. In this particular embodiment, the balloon 802 is inflatable to secure tissue between the balloon and the apparatus, thus facilitating delivery of sutures and/or clips through the tissue. Use of the balloon 802 may improve consistency and repeatability of suture and/or clip delivery since the targeted tissue may be grasped prior to engagement by the suture and/or clip. At least a portion 804 of the balloon 802 may optionally be covered with a material, such as but not limited to Kevlar, DARON™, Dacron™, a firm rubber substance, GORTEX™, any combination of the above, or similar substances to prevent clips or penetrating members from bursting the balloon during delivery into the tissue. In this embodiment, a Kevlar shield 804 may optionally be used with the balloon 802. As seen in FIG. 12, a luer lock 806 may provided to enable inflation and/or deflation of balloon 802. It should be understood that during delivery, the balloon 802 may be in an uninflated condition to facilitate entry and positioning of the balloon. In this embodiment, a screw locking mechanism 807 may used for balloon apposition to the annulus or tissue T. This may occur during, before, or after inflation of balloon 802.

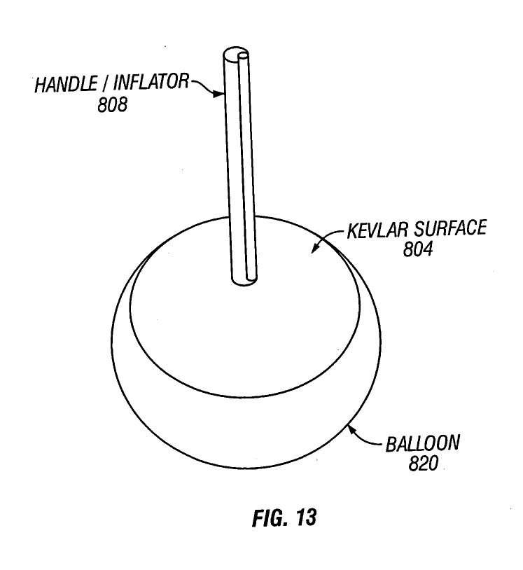

FIG. 13 provides an isolated view of just the balloon 802 in an inflated condition. As seen in FIG. 13, needle or fastener proof surface 804 may optionally be provided on the balloon 802. A handle and/or balloon inflator 808 is also provided to enable positioning and inflation of the balloon. Further details of this and other suitable devices can be found in commonly assigned, copending U.S. patent application Ser. No. ______ (Attorney Docket No. 40405-0002) filed Nov. 13, 2003, fully incorporated herein by reference for all purposes. The anvil or balloon 802 may optionally be used to bring tissue together and facilitate delivery of fasteners into the tissue. In one embodiment, the anvil 802 may also allow the embodiment to deliver the fasteners without having the device expand radially outward as shown in FIG. 4B to engage tissue.

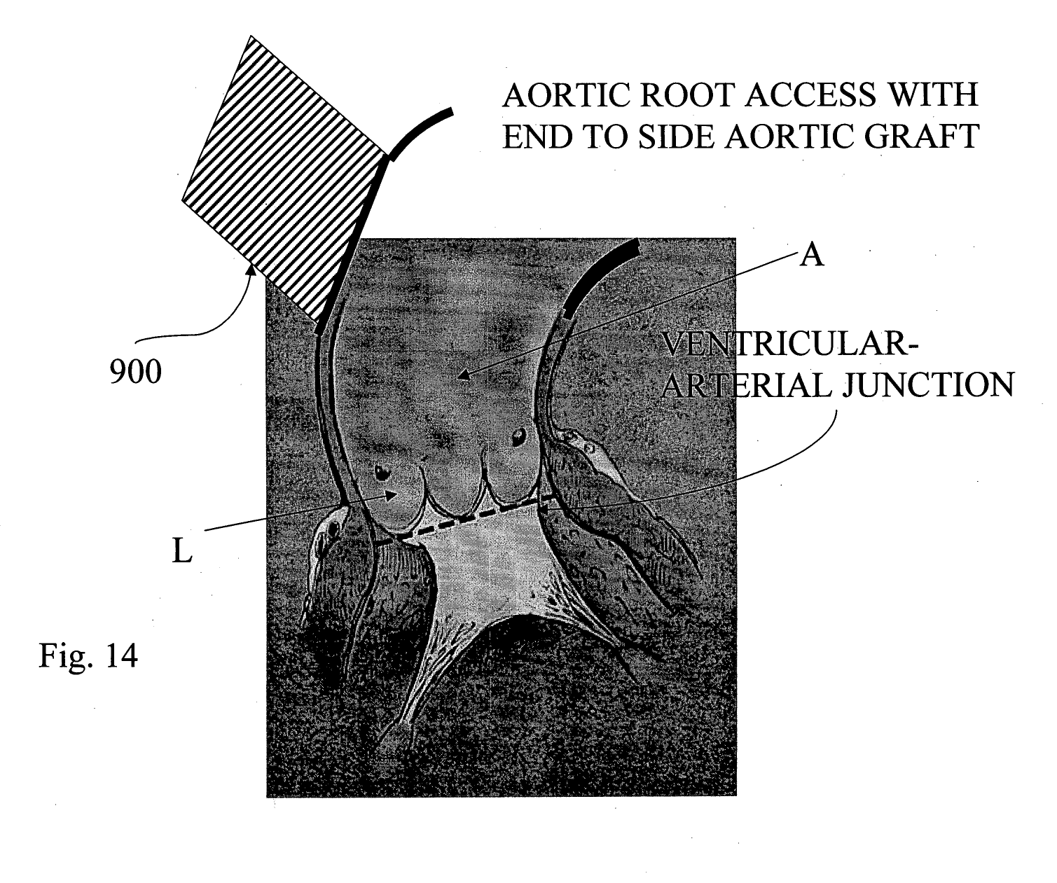

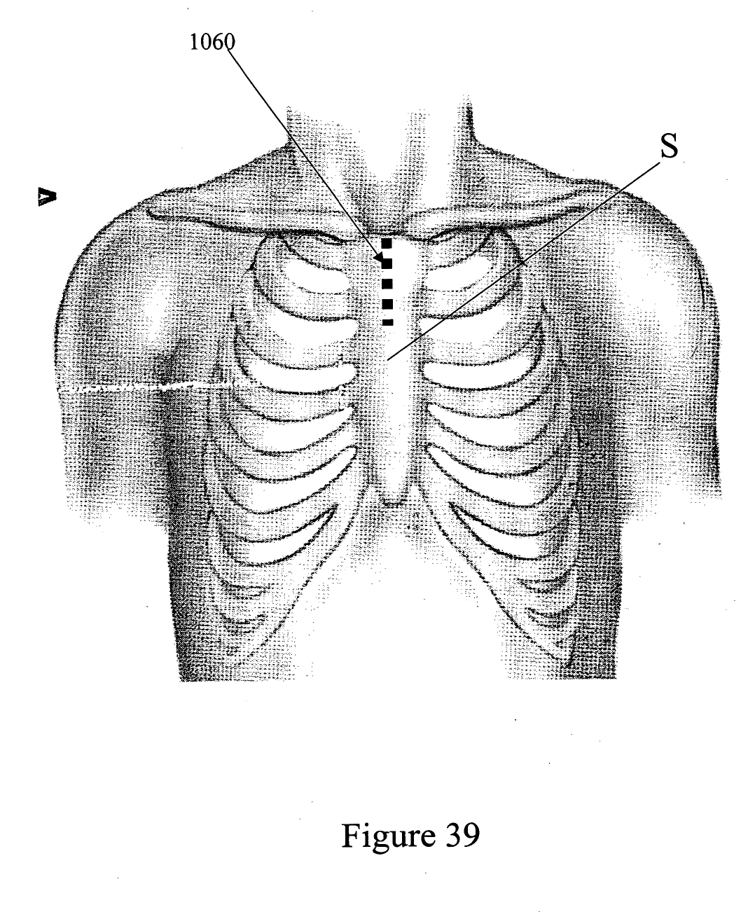

Referring now to FIG. 14, another aspect of the present invention will now be described. This aspect of the invention provides devices and methods for off-pump aortic valve replacement. In one embodiment of a method for off-pump valve replacement, the first step or Stage I comprises accessing to the aortic root A. As will be described in FIG. 39, the access to the aortic root A may use a significantly smaller incision that does not involve cutting the entire sternum.

In this embodiment of the invention, access to the aortic root A is accomplished by attaching a Dacron™ graft 900 of appropriate size such as but not limited to about 25 to 31 mm, onto the ascending aorta in an end-to-side fashion (FIG. 14). It should be understood that the graft is not limited to Dacron™ and other materials such as nylon, polymeric material, other materials listed herein, combinations of materials, or the like may also be used. It should also be understood that the graft may also be sized to accommodate much smaller devices. In one nonlimiting example, the graft 900 may have a diameter of about 8 to 25 mm. Some may have a 10-12 mm diameter to accommodate expandable valve prosthesis which may be designed for delivery through 10 mm openings. The graft 900 may also be considered one embodiment of a blood containment device that prevents the loss of blood from the blood vessel during an off-pump procedure. The graft 900 is not limited to use on the aorta and may be used on other blood vessels throughout the body such as but not limited to the femoral artery, the lower aorta, or the like.

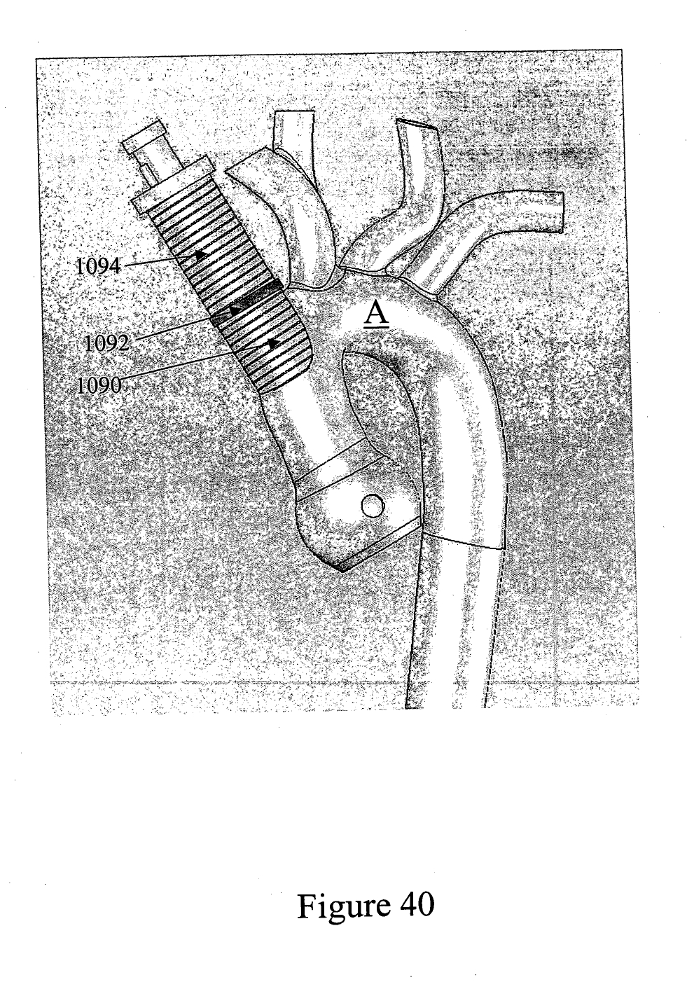

In one embodiment, the attachment of graft 900 may occur by placing a side-biting clap on the ascending aorta and sewing the graft 900 with polypropylene suture to the aorta or by using a graft-fastening device where the graft is attached in one-fell swoop and does not use side biting of the aorta. As described in regards to FIG. 40, a multi-fire device may also be used to attached the graft 900 to the aorta.

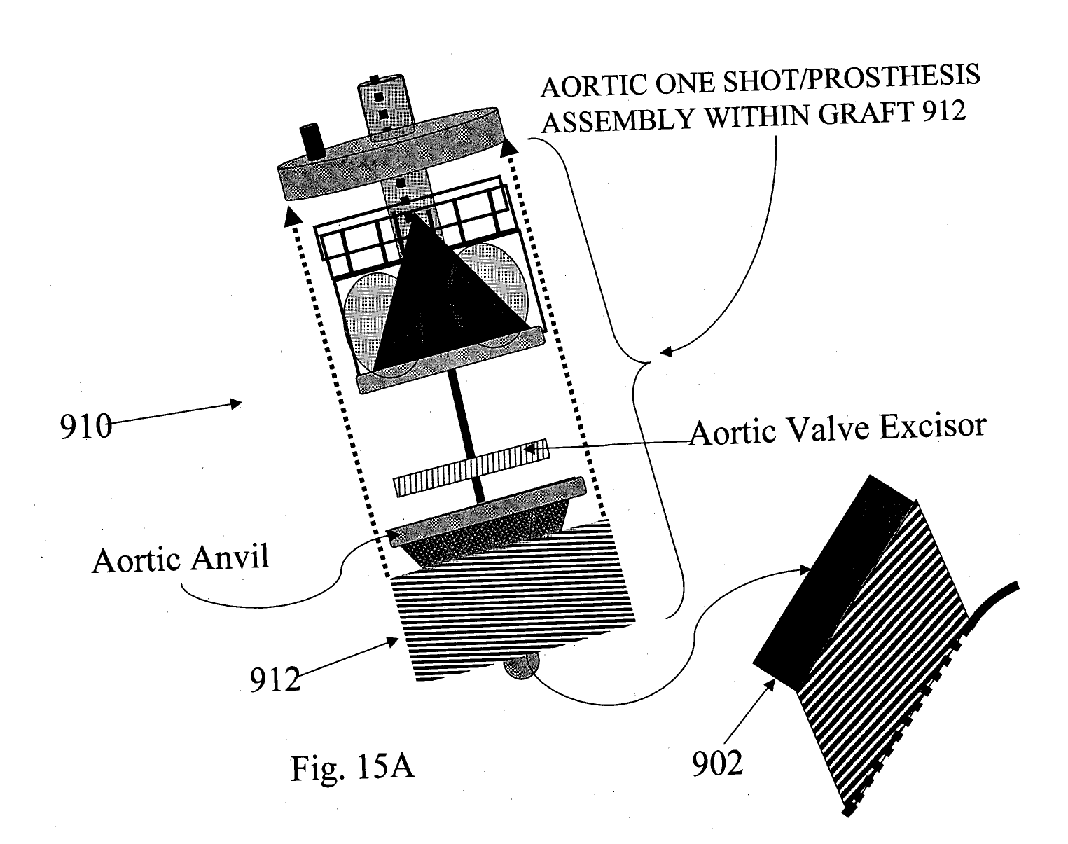

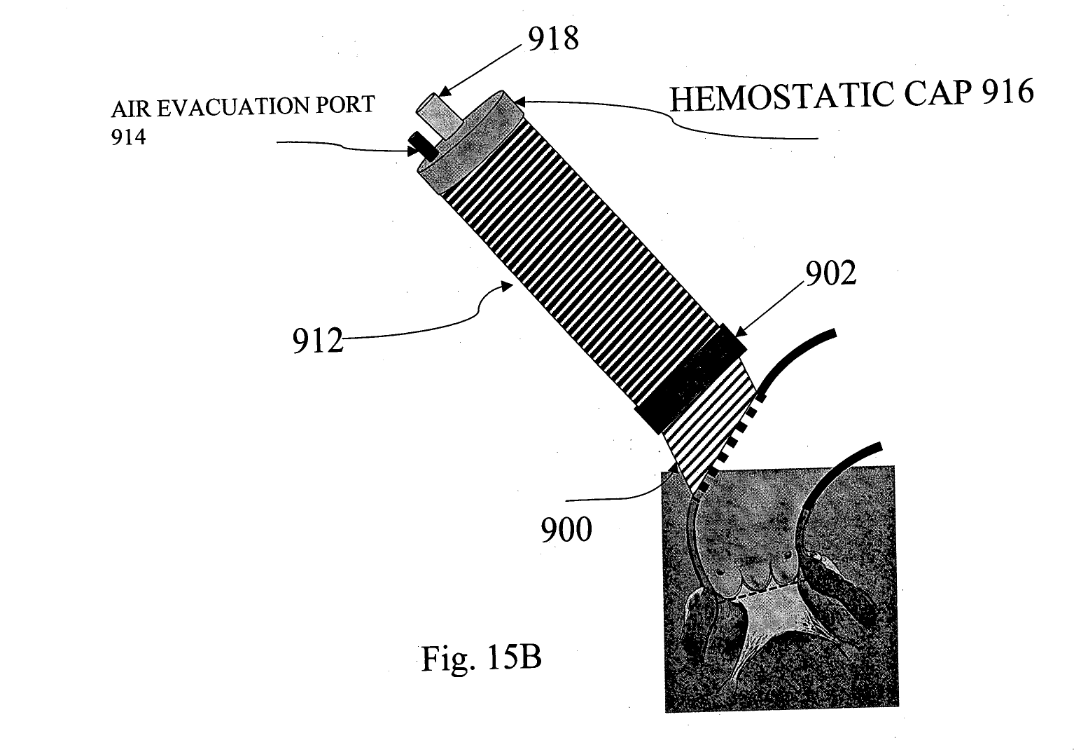

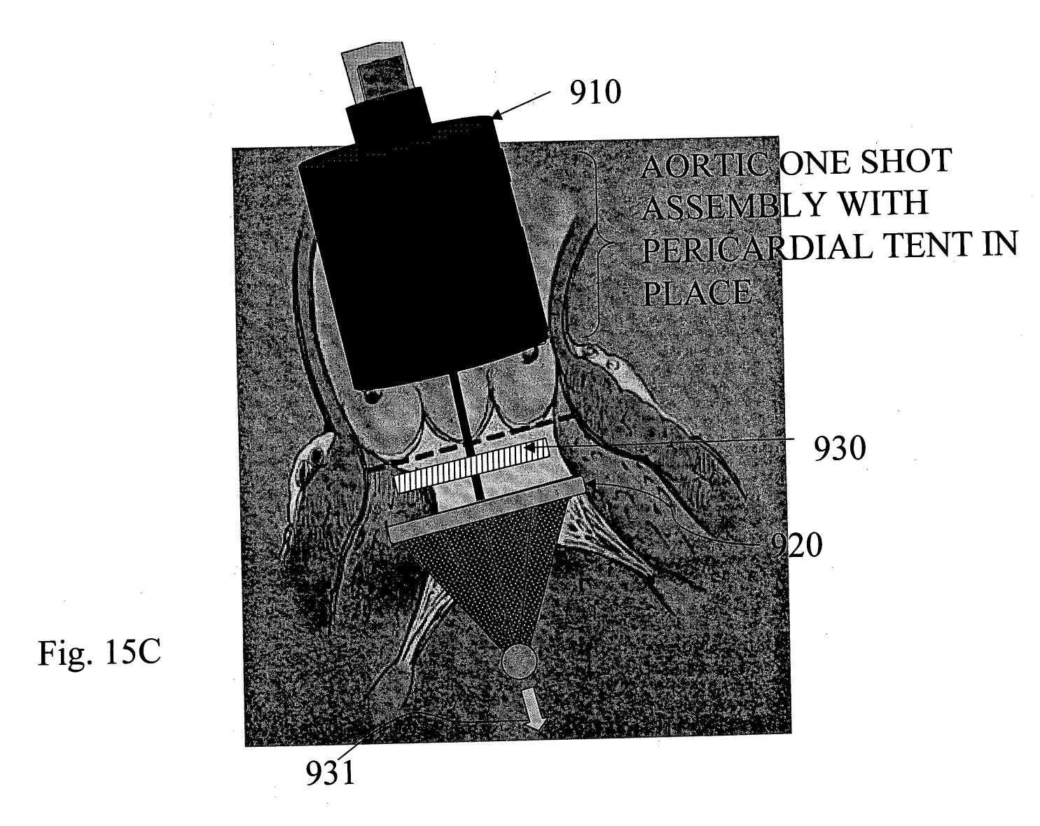

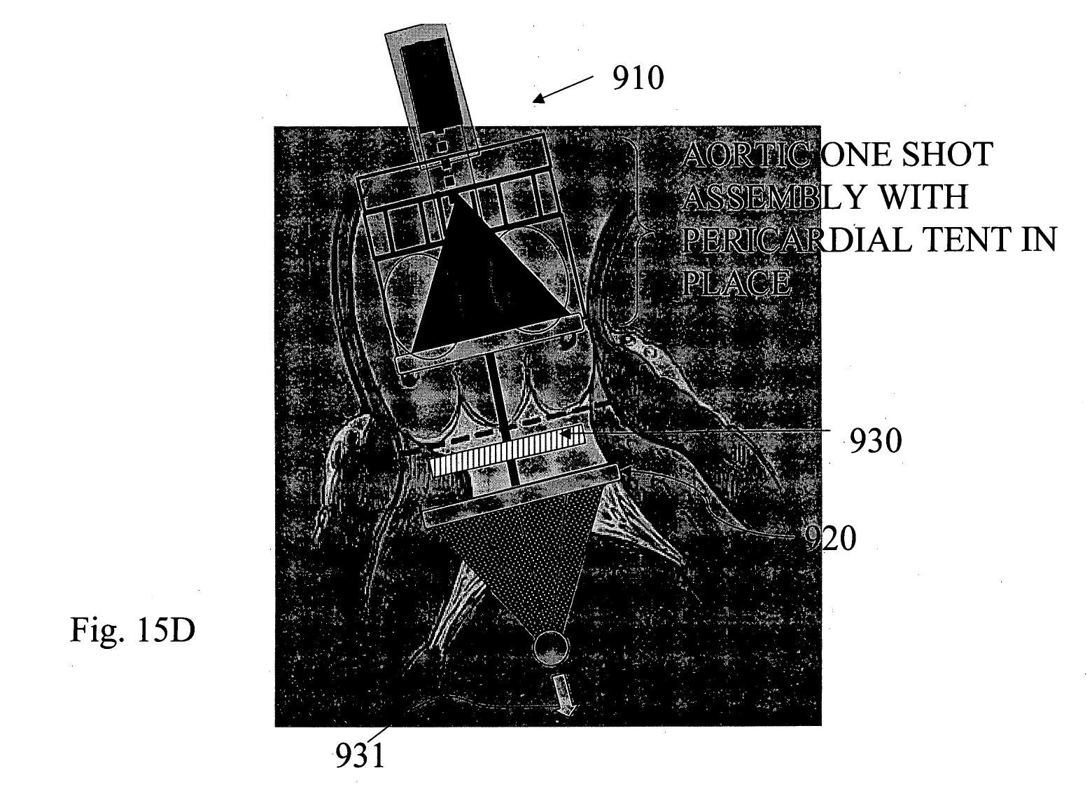

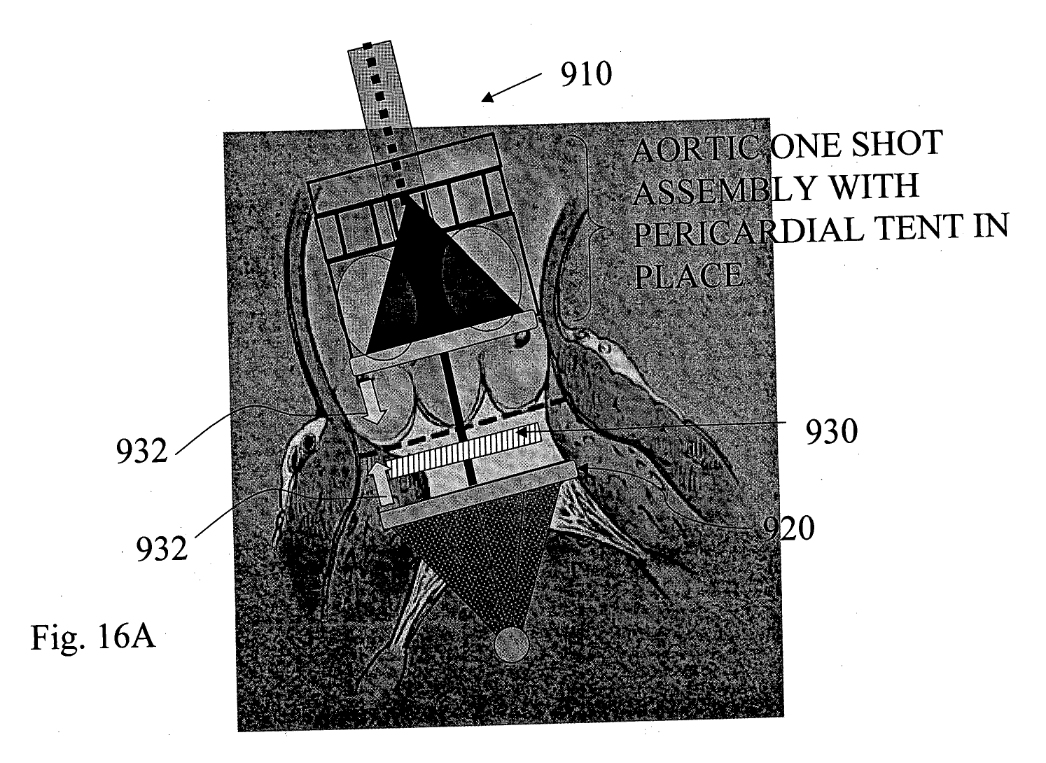

Referring now to FIGS. 15A and 15B, the second step or Stage II of the method comprises engagement of aortic cone anvil. In this embodiment of the invention, once the graft 900 is attached to the ascending aorta, a clamp 902 is placed across the Dacron™ graft 900. In this embodiment, the aortic one shot assembly 910 which may be pre-inserted into a second Dacron™ tube graft 912 is then attached to the graft 900 that has been previously attached to the ascending aorta, as seen in FIG. 15B. It should be understood, in other embodiments, the one shot assembly 910 may be included in the first Dacron™ graft 900. As will be described in the description of assembly 910, a “Screw on” type device, in an end-to-end fashion, facilitates this attachment. The assembly 910 may optionally include an air evacuation port 914 and a hemostatic cap 916 at the proximal end of the graft 912. The port 914 may be used to bleed off any air that may remain in the graft 912. An opening 918 may optionally be included to allow a shaft or other extension from the assembly 110 to extend outward from the graft 912. In the present embodiment, the assembly 910 also includes the aortic anvil 920 and an aortic valve excisor 930. The anvil 920 may be viewed as a valve leaflet support or engagement device. Once attached, the clamp 902 on the Dacron™graft stump is then removed and blood is allowed to fill the rest of the Dacron™tube graft 912. The air evacuation port 914 is used to remove air from within the Dacron™ tube. This completes access to the ascending aorta.

Referring now to FIGS. 15C and 15D, the assembly 910 is advanced through the graft and into the ascending aorta to position the assembly 910 to deploy the valve prosthesis. As seen more clearly in FIG. 15D (with the outer portion of assembly 910 removed for ease of illustration), engagement of aortic cone anvil will comprise advancing the aortic anvil 920 and the excisor 930 through the aortic outflow tract. First, the assembly 910 is advanced in the direction indicated by arrow 931, toward the aortic root A. Under echocardiography, fluoroscopic guidance, or other forms of visualization, the aortic valve cutting element 930 and anvil 920 are advanced through the native aortic valve. The aortic anvil 920 is advanced through the aortic outflow tract and seated below the aortic annulus.

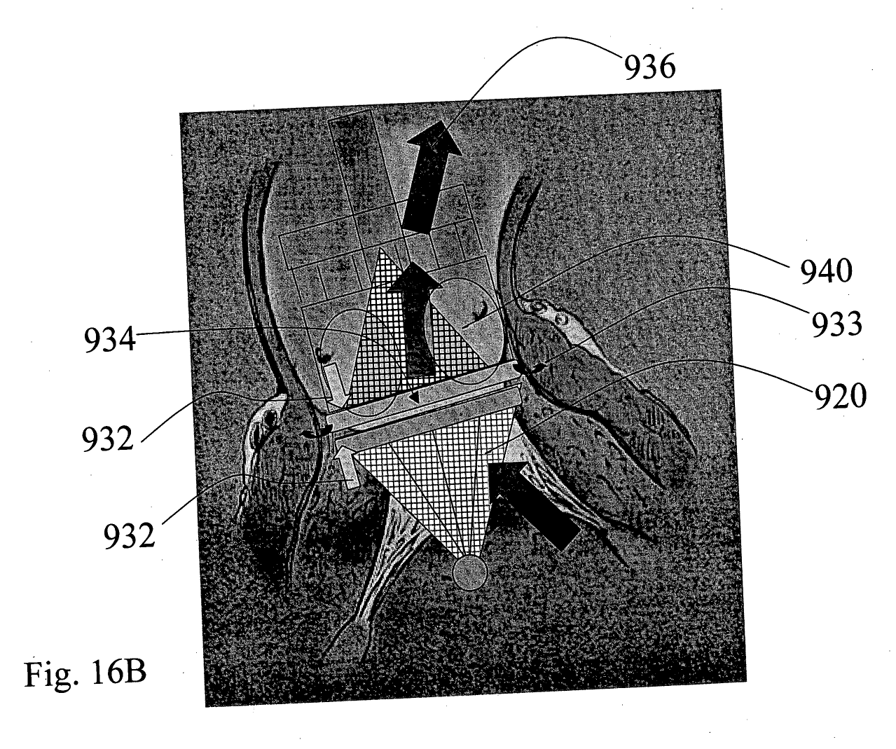

Referring now to FIGS. 16A and 16B, Stage III of the method comprises deployment of aortic valve prosthesis. Referring now to FIG. 16A, the elements of the assembly 910 and the aortic anvil 920 are brought together as indicated by arrows 932. Once the aortic anvil 920 is engaged and seated below the aortic annulus, the aortic valve and fastening device is advanced onto the native aortic valve in an orientation determined by the aortic anvil 920. Fasteners 933 and the stented bioprothesis 934 on assembly 910 may be then be delivered in method as described in U.S. patent Application (Attorney Docket No. 40450-0002). Although not limited to the following, the fasteners 933 may be of the designs set forth in U.S. patent Application (Attorney Docket No. 40450-0002) and included herein by reference. Prior to deployment of the aortic valve prosthesis 936, adenosine phosphate may be administered intravenously or through some other method to the patient. This will cause temporary asystole while the aortic valve prosthesis and fasteners are deployed. In some embodiments, this should take less than 60 seconds. It should be understood that other methods and/or medications which can cause this temporary asystole may also be used in conjuction with the present invention. During this time blood flow will continue to flow as indicated by arrows 936 but a reduce rate and pressure.

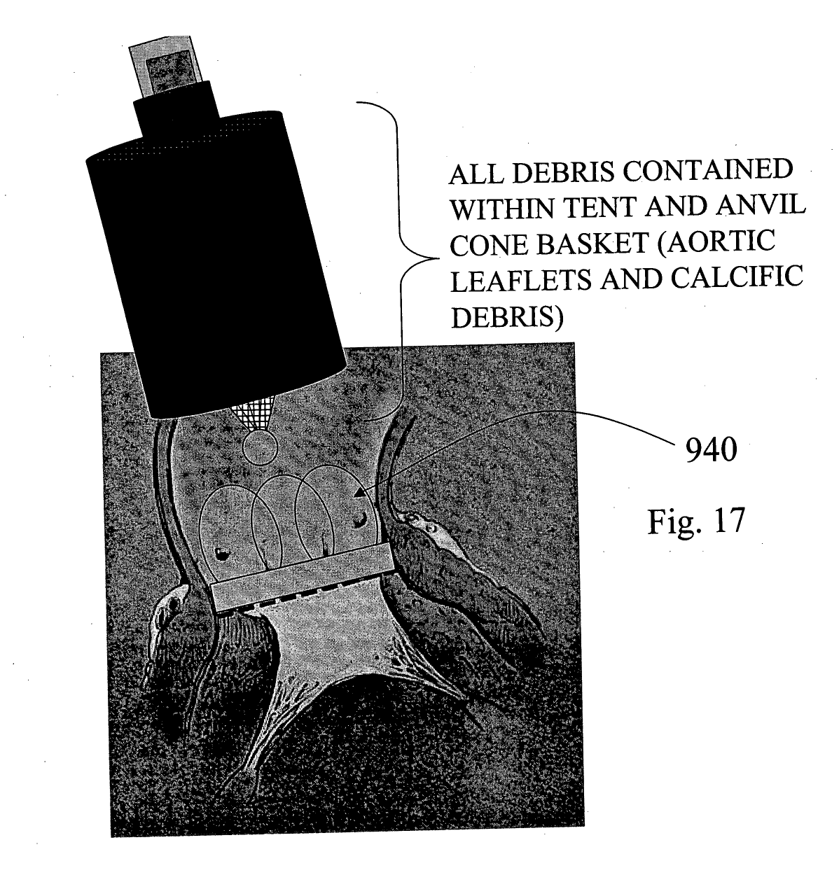

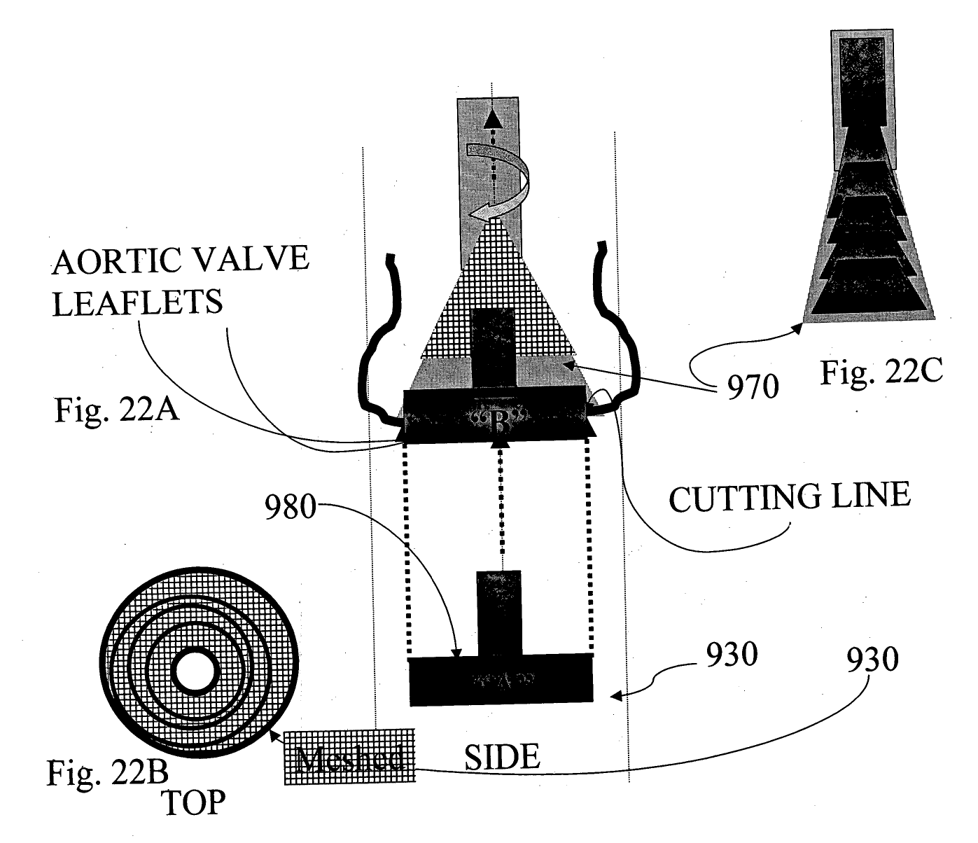

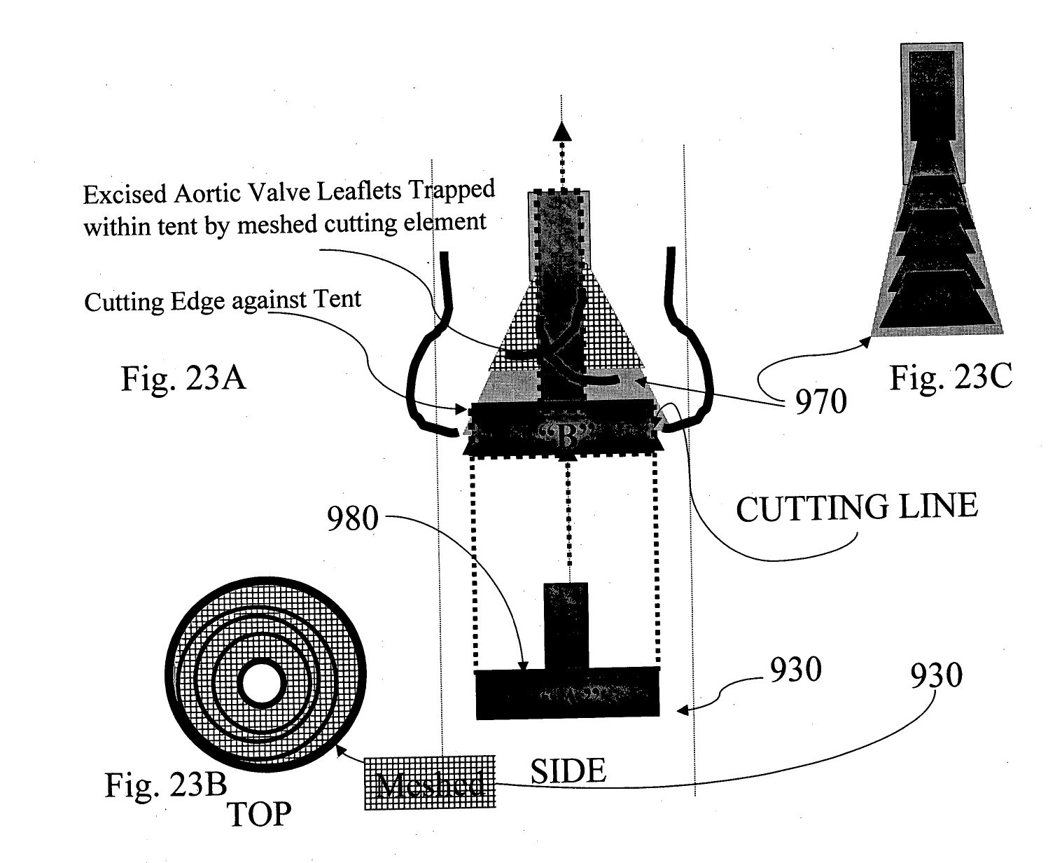

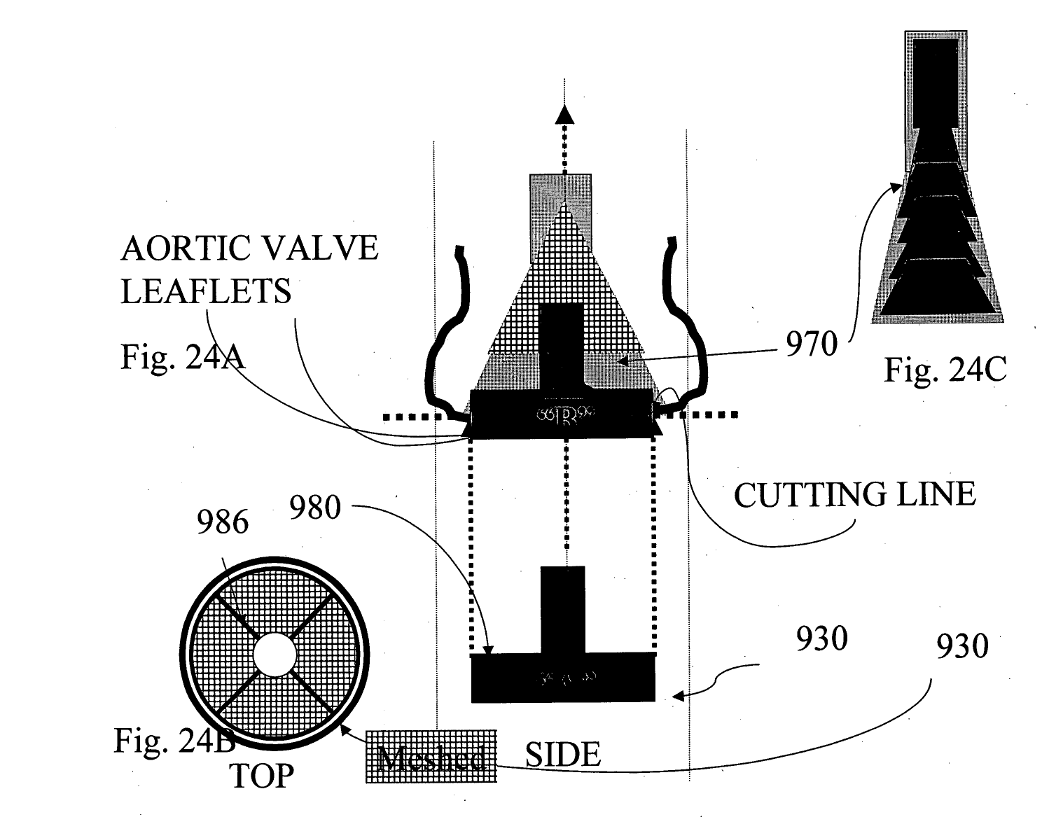

Referring now to FIG. 17, Stage IV of the method comprises extraction of assembly 910. After the aortic valve prosthesis 936 and fasteners 933 have been deployed, the cutting element 930 is turned in a clockwise or counter clockwise fashion with simultaneous proximal counter traction. This will result in excision of the three aortic leaflets L. The bioprosthetic aortic valve leaflets 940 are kept out of harms way of the cutting element by the pericardial metal ribbed tent 970 as described in more in FIGS. 22 and 23. In one embodiment of the present invention, once the valve is seated or anchored, the cutting element 930 is retracted up against the tent and by turning the cutting element this results in cutting the valve leaflets. Because the cutting element 930 may have a mesh undersurface, the leaflets are trapped within the tent above and the cutting element below. The trapped leaflets are then extracted within the entire assembly. Typically, cutting occurs after the valve prosthesis is attached to tissue. In some alternative embodiments, cutting may occur first, with valve attachment coming second. The aortic leaflets are retracted up into the pericardial tent 950 and other debris is collected in the aortic cone anvil. All components, i.e. aortic anvil 920, cutting element 930, pericardial tent 970 and fastening device are retracted as one unit into the Dacron™ tube.

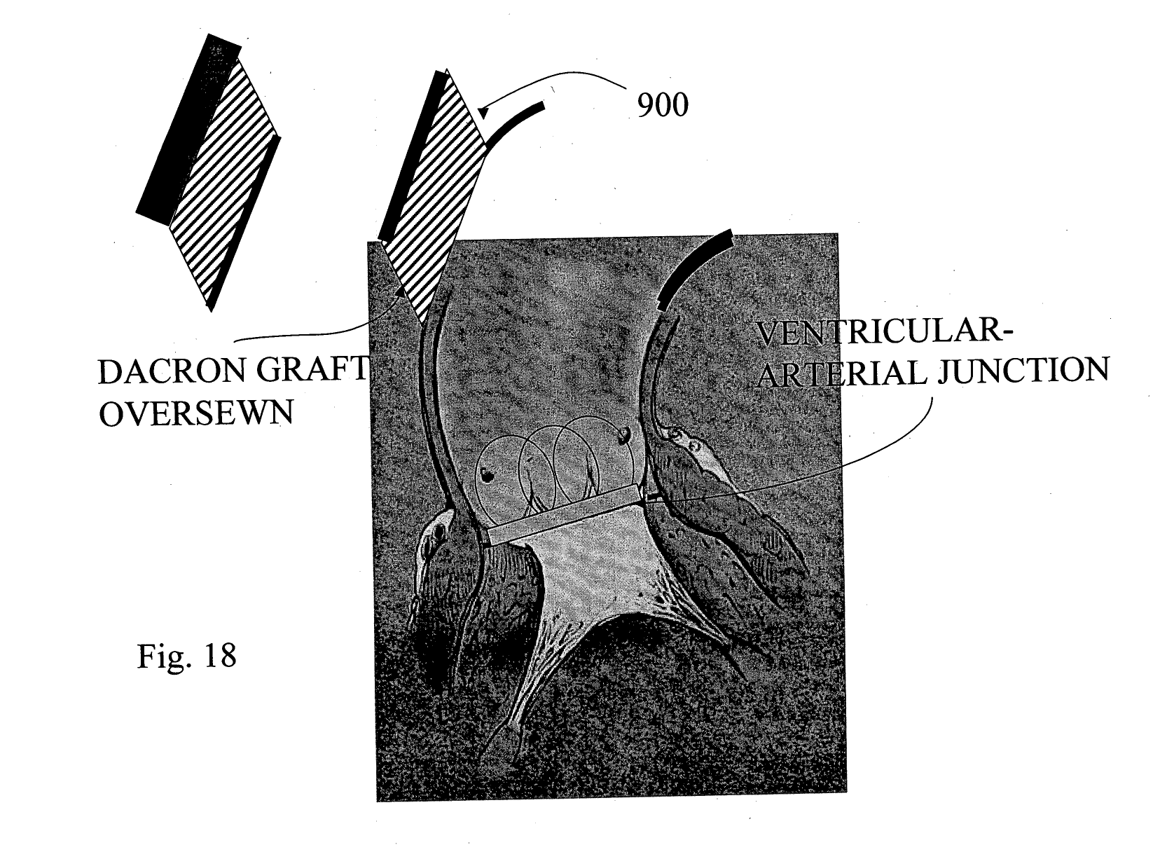

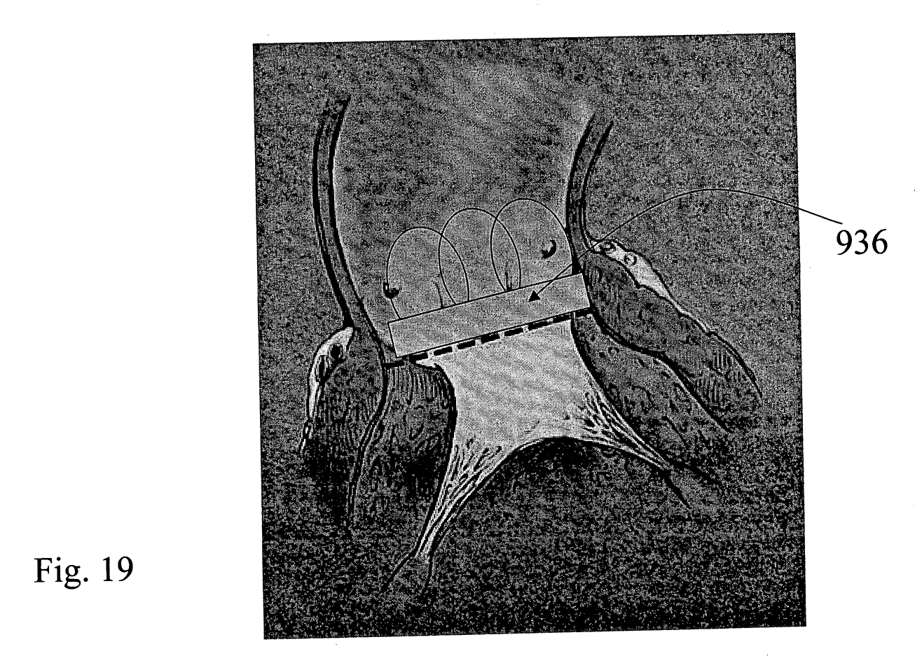

Referring now to FIGS. 18 and 19, Stage V of the method comprises closure of the aortic access. FIG. 18 shows that the Dacron™ graft 900 attached to the aorta is clamped and over sewn to close the access. FIG. 19 shows the completed procedure with the bioprosthetic aortic valve 936 in position (for ease of illustration, fasteners are not shown). At the conclusion of the procedure, an echocardiogram may be performed to assess valve function.

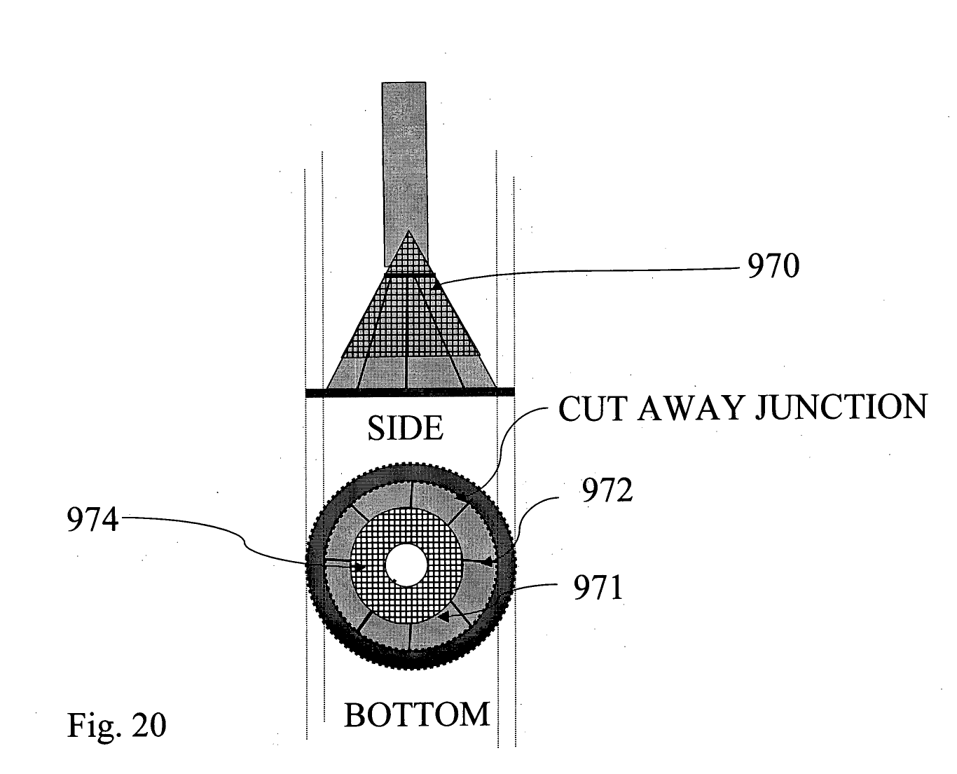

Referring now to FIGS. 20-26, embodiments of the assembly 910 used to perform the above procedure will now be described. One embodiment of the assembly 910 includes the following elements: aortic one shot fastener (previously described), pericardial tent 950, aortic leaflet cutting element 930 (two designs), and inverted umbrella aortic cone anvil 920 (previously described). The following will describe the components that make up the assembly 110 for deploying a stented bioprosthetic aortic valve 936 except for the aortic one-shot fastener and inverted aortic cone anvil, which have been described previously.

FIG. 20 shows one embodiment of a pericardial tent 970. The pericardial tent 970 of FIG. 20 is a cone shaped device of appropriate size that slides into the outflow tract of the prosthetic aortic valve 936. It is situated in this location to provide protection to the prosthetic valve leaflets during excision of the native aortic valve leaflets by the cutting element. The cone 970 may optionally be attached to the aortic valve prosthesis 936 on the inferior surface of the prosthetic annulus. When the cutting element 930 is used to cut the native aortic leaflets and the element 930 is extracted, the tent 970 would separate from its prosthetic attachment when the assembly 910 is withdrawn.

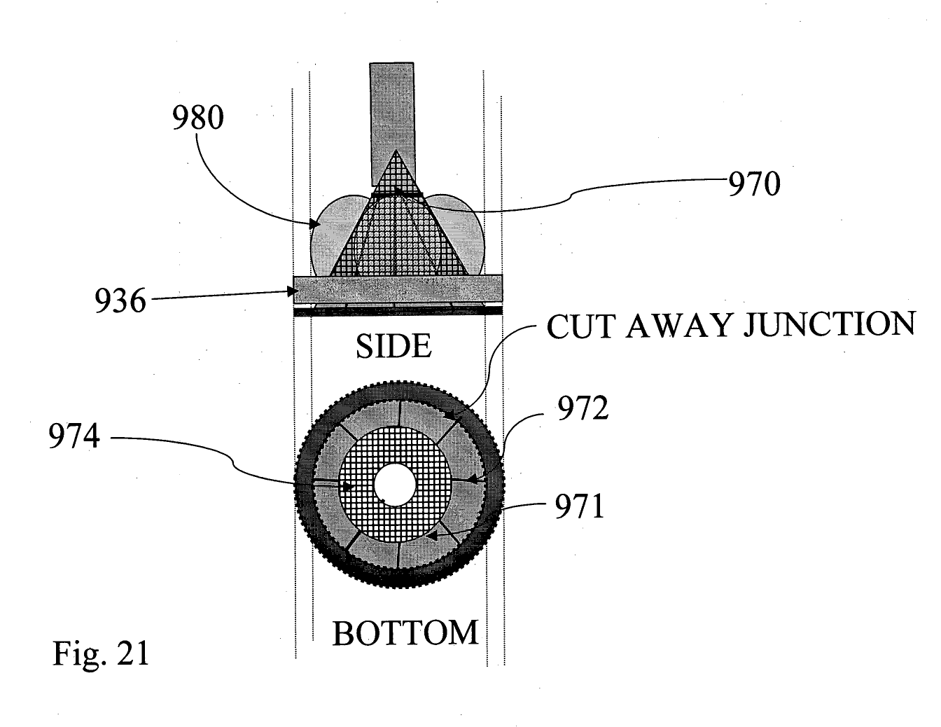

The tent 970 may optionally be constructed of a variety of materials including but not limited to pericardium 971, polytetrafluoroethylene (Gortex™) or a soft pliable rubber material. This material may be overlaid onto wire frame. The pericardial tent 970 may optionally include ribs or wires 972. The upper part 974 of the cone would be constructed of a soft mesh material to allow blood flow to pass through. FIG. 21 shows the pericardial tent 970 with the bioprosthesis 980 in place.

Referring now to FIGS. 22A-22C, one embodiment of the aortic leaflet cutting element 930 will be described. The cutting element 930 may be a circular cutting device used to excise the native aortic leaflets L. As a nonlimiting example, the element 930 may optionally be constructed of a flexible metallic material with a sharp cutting edge 980. The element 930 may be a component of the assembly 110 that would excise the valve when the assembly is about to be retracted (or at any other time as desired). As seen in FIG. 23A-23C, by applying proximal traction and a circular rotating motion on the cutting element handle, the valve leaflets L would be excised as the assembly 910 is retracted. The leaflets L may be captured between the pericardial tent 970 and cutting edge 980 of the cutting element 930. The engagement of the leaflets and the motion of the cutting edge will excise the leaflets L. As seen in FIG. 23B, the interior of the cutting element 930 may be meshed or otherwise configured to prevent the excised valve leaflets L from escaping into the blood vessel.

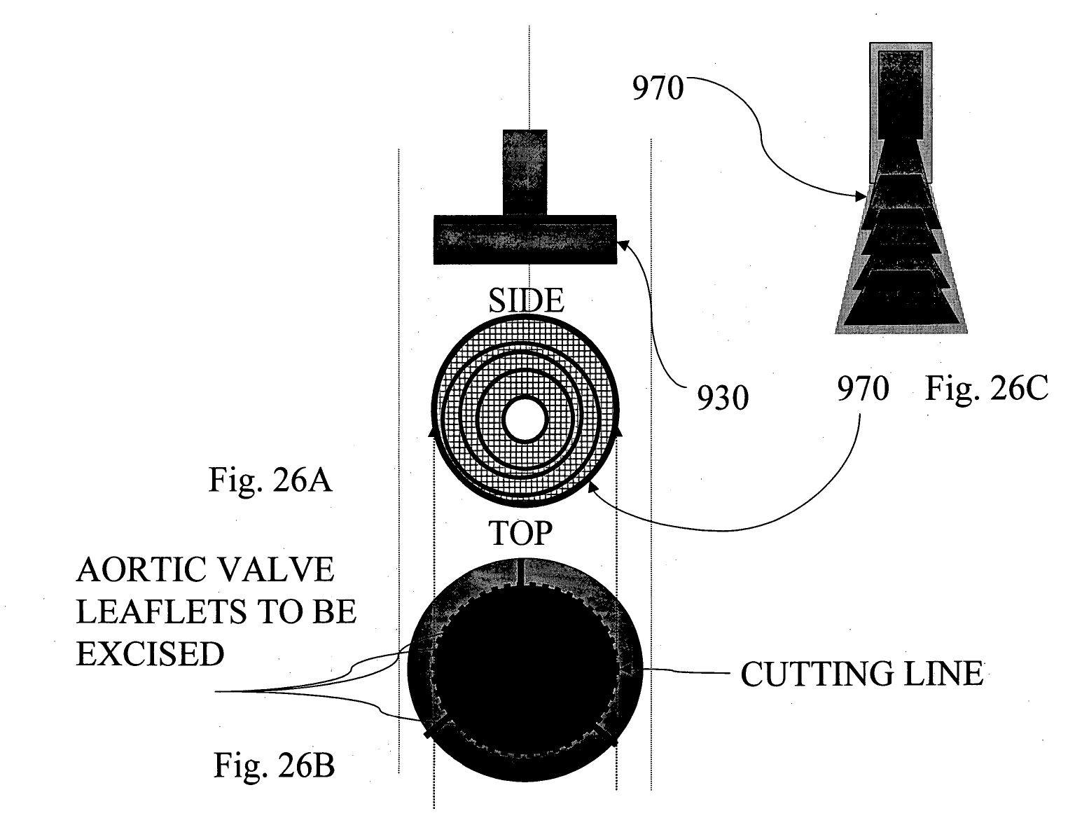

In the present embodiment, cutting occurs by rotating the cutting element 930 against the valve leaflets and the pericardial tent. FIG. 23 shows the cutting element 930 advance from positions “A” to “B”. By applying rotational motion on the cutting element against the tent, the valve leaflets are cut and become trapped within the cutting element and the tent. FIG. 23B Illustrates the mesh at the bottom of the cutting element and FIG. 23C shows how the cutting element collapses when retracted up into the tent with the leaflets confined to the assembly. FIGS. 24A, B and C illustrate the same embodiments as in FIGS. 24A, B and C (except for 24B) illustrates a different cutting element design. FIGS. 26A, B and C illustrate the three major components of the cutting element but specifically how they relate to the cutting of the aortic leaflets. For ease of illustration, the aortic valve is not shown in the previous FIGS. 23 and 24. It should be understood that some embodiments may use a device similar to that of FIG. 3A-3D to remove the leaflets. FIG. 2 are also relevant to supporting valve leaflets for placement of the prosthesis and/or valve leaflet removal.

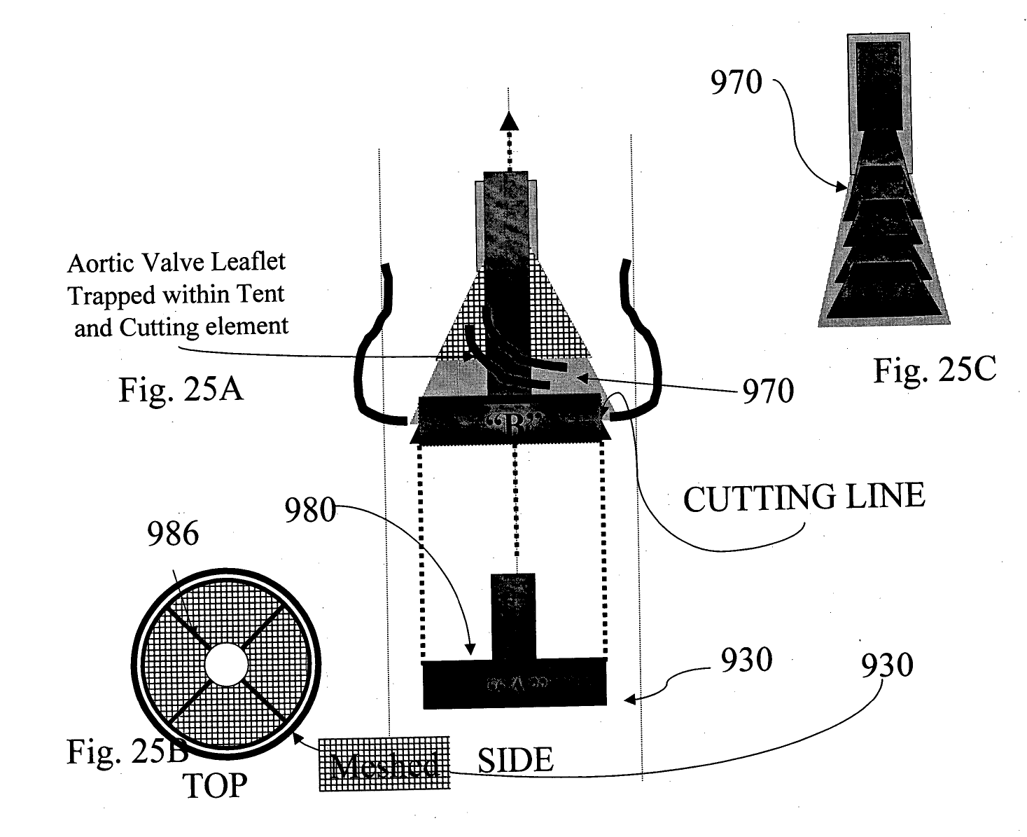

FIGS. 24 to 25 show another embodiment of the cutting element 930. As seen more clearly in FIGS. 24B and 25B, the meshed portion in the interior of the cutting element 930 has a ribs 986 in the mesh.

FIGS. 26A-26C show where the cutting elements will engage the valve leaflets L to excise them. Specifically, FIG. 26B shows the cutting line where the cutting element will excise the leaflets.

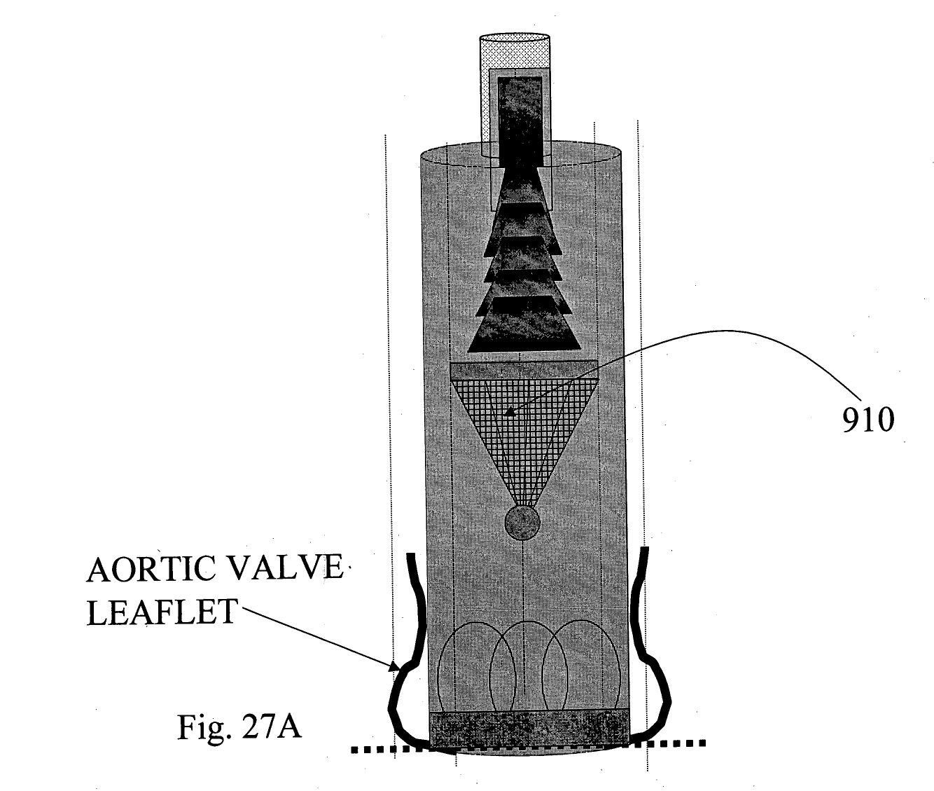

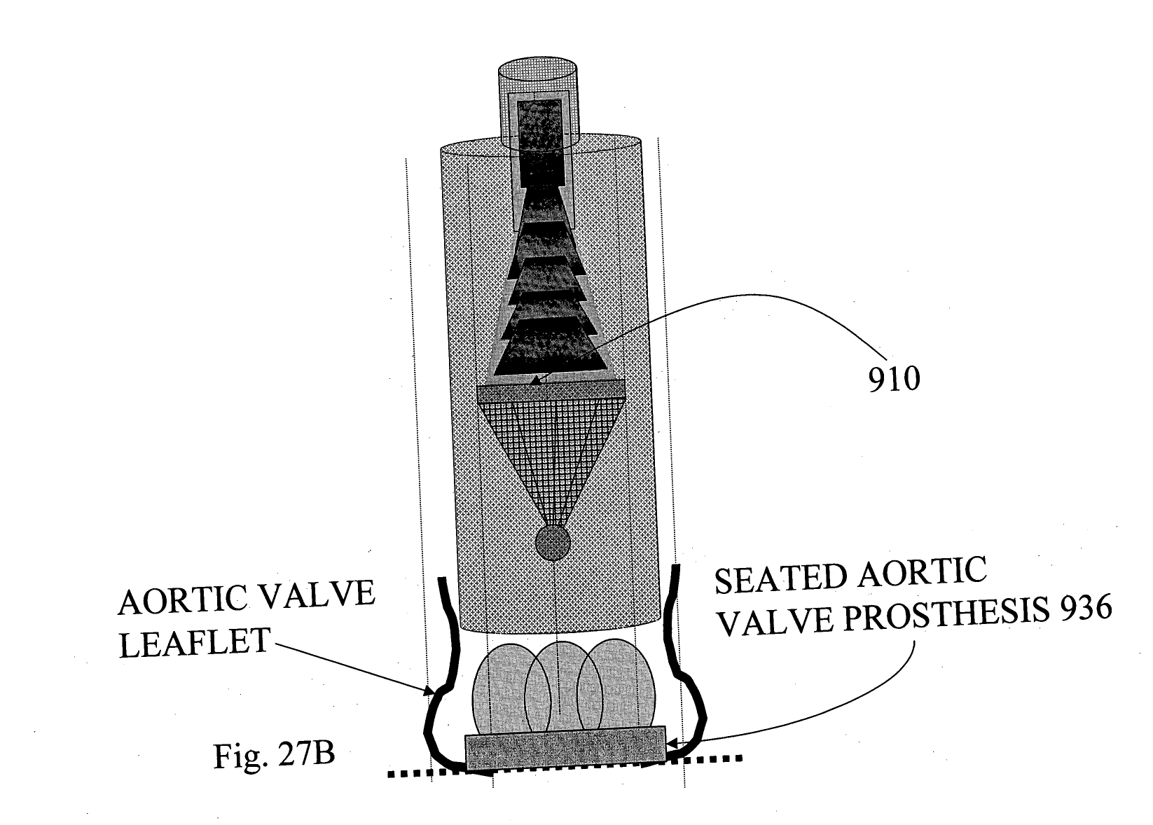

Referring now to FIG. 27A, the entire assembly 910 is shown positioned to engage the valve leaflets L. FIG. 27B shows the valve prosthesis seated in position and the assembly 910 being withdrawn.

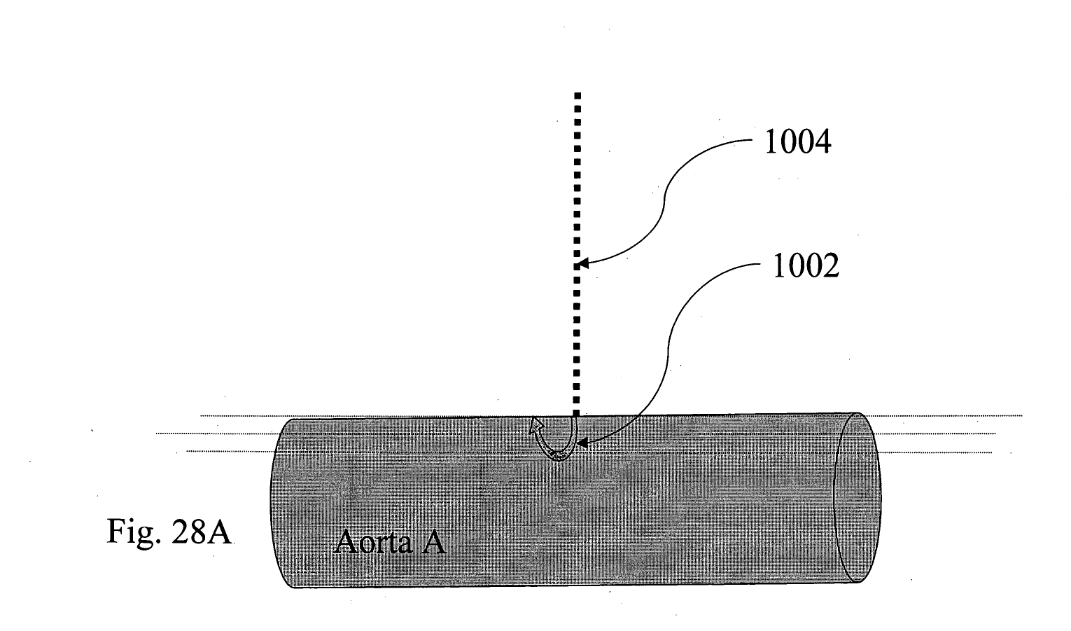

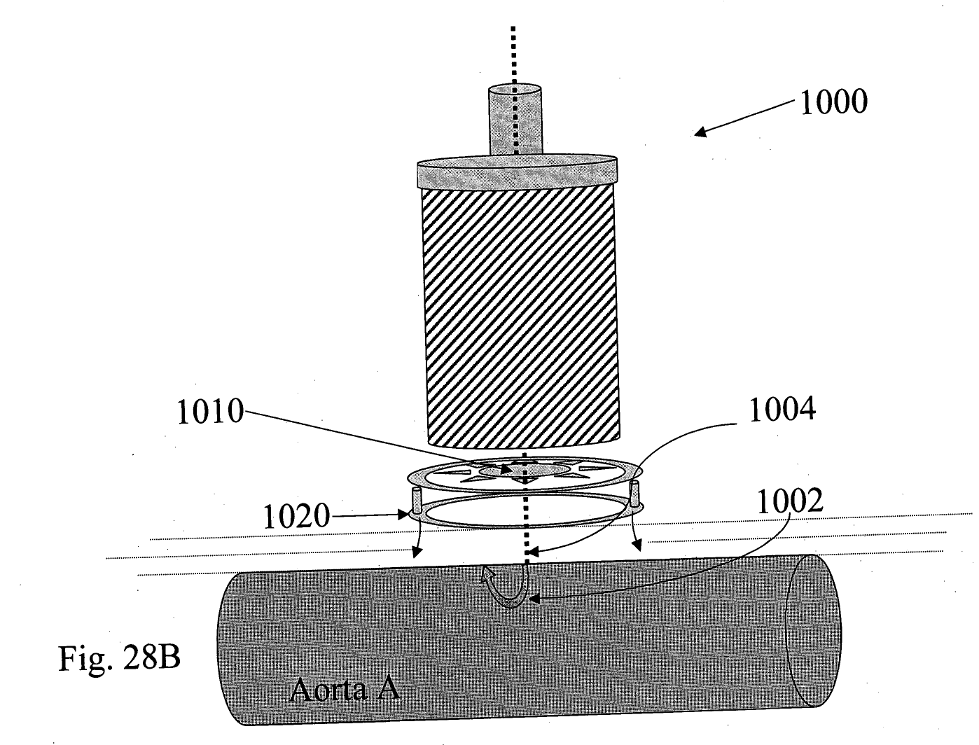

Referring now to FIGS. 28-37, a still further aspect of the present invention will now be described. This aspect of the present invention provides an end-to-side anastomosis device 1000 for attaching a graft to the aorta or other vessel. Referring now to FIG. 27, one embodiment of the method and device for providing graft attachment will now be described.

As seen in the embodiment of FIG. 28A, a needle 1002 is provided with a guide wire 1004 attached to the needle. The needle may have a variety of shapes and in some embodiments may be made of a shape memory material. In the present embodiment, the needle 1002 is curved. The needle 1002 is advanced into the aorta A as seen in FIG. 28A.

Referring now to FIG. 28B, the needle 1002 anchors the guide wire 1004 to guide the device 1000 to the target site on the aorta A. As seen in the FIG. 28B, the device 1000 includes a first portion 1010 of a tissue clamp and seating rings 1020 whose functions will be described in more detail below.

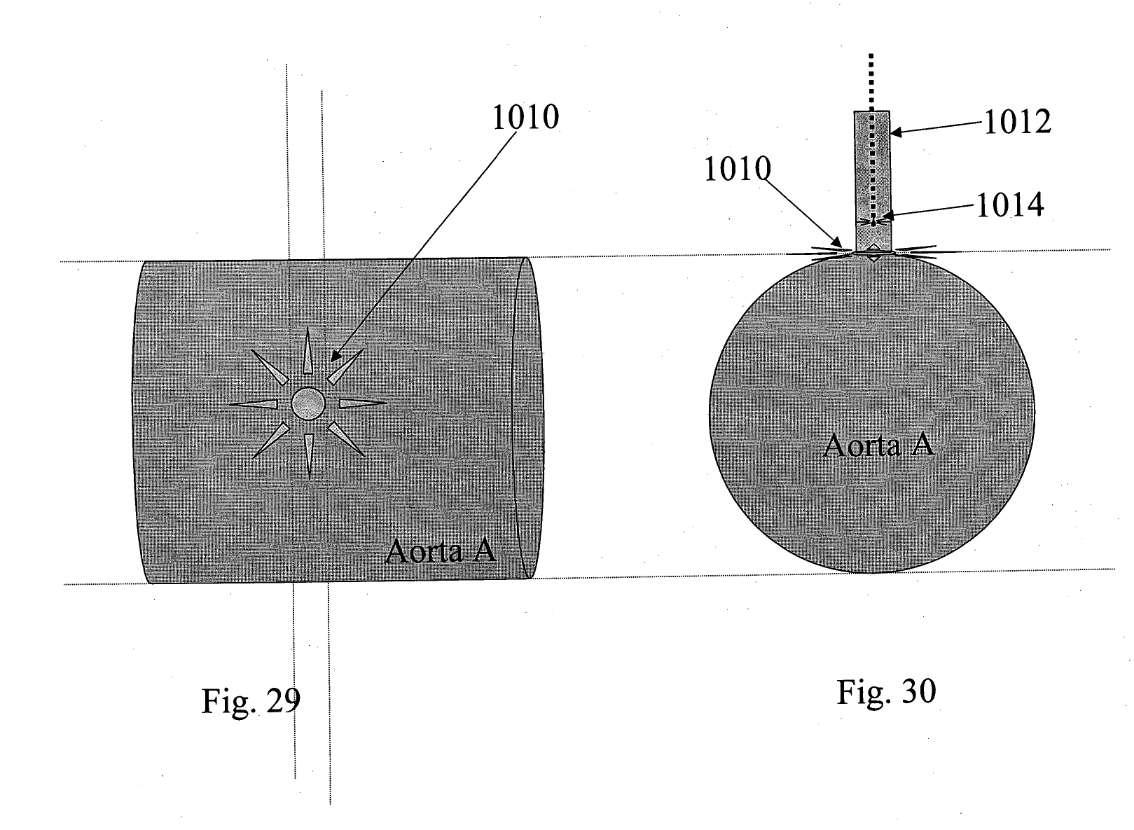

Referring now to FIGS. 29-30, the first portion 1010 of a tissue clamp is guided into place on the vessel surface, which in this case is on the aorta A. FIG. 29 is a top-down view of the first portion 1010 engaging the aorta A. For ease of illustration, the graft and other portions of the device 1000 are not shown. FIG. 30 is a side, cross-sectional view with the first portion 1010 shown seated against an outer surface of the aorta A. FIG. 30 also shows that the portion 1010 may be coupled to a hollow shaft 1012 that allows for the delivery of a second portion 1014 of the tissue clamp towards the aorta A. The second portion 1014 may have a variety of configurations. In the present embodiment, portion 1014 is a device expandable into an enlarged configuration. The enlarged configuration is similar in shape to that of the first portion 1012. In its unexpanded configuration, the second portion 1014 may be sufficiently sharp or have cutting surfaces sufficient to penetrate through the walls of the blood vessel. In some other embodiments, a stylet or trocar may be used to make a small penetration allowing the second portion 1014 to be delivered into the aorta A.

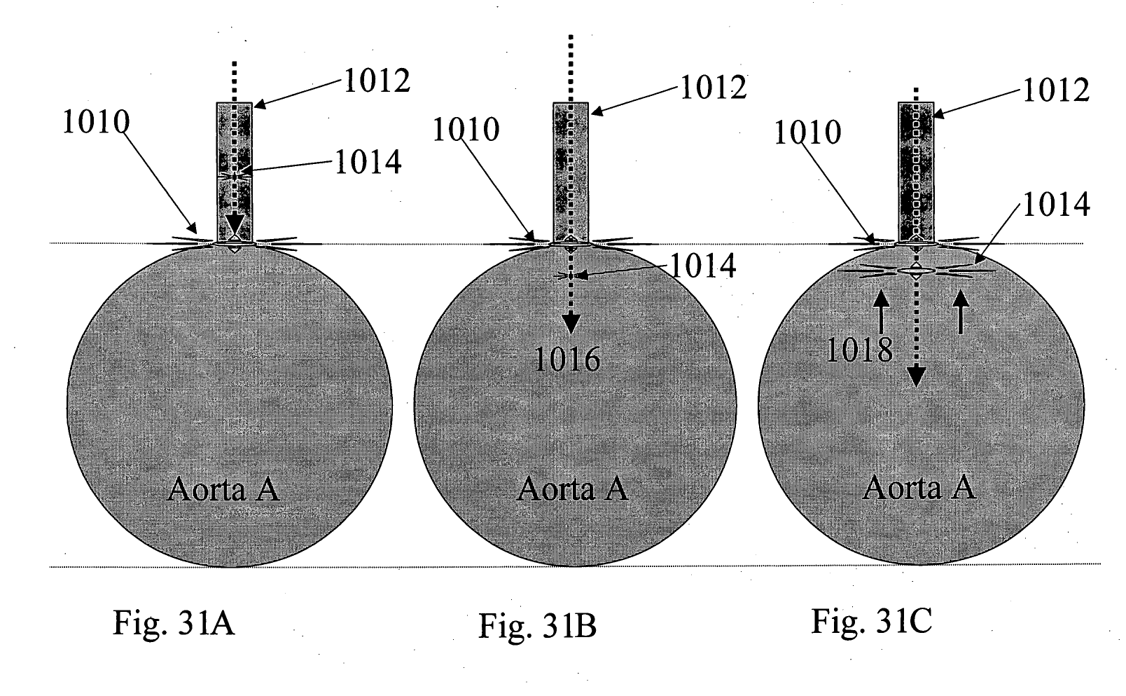

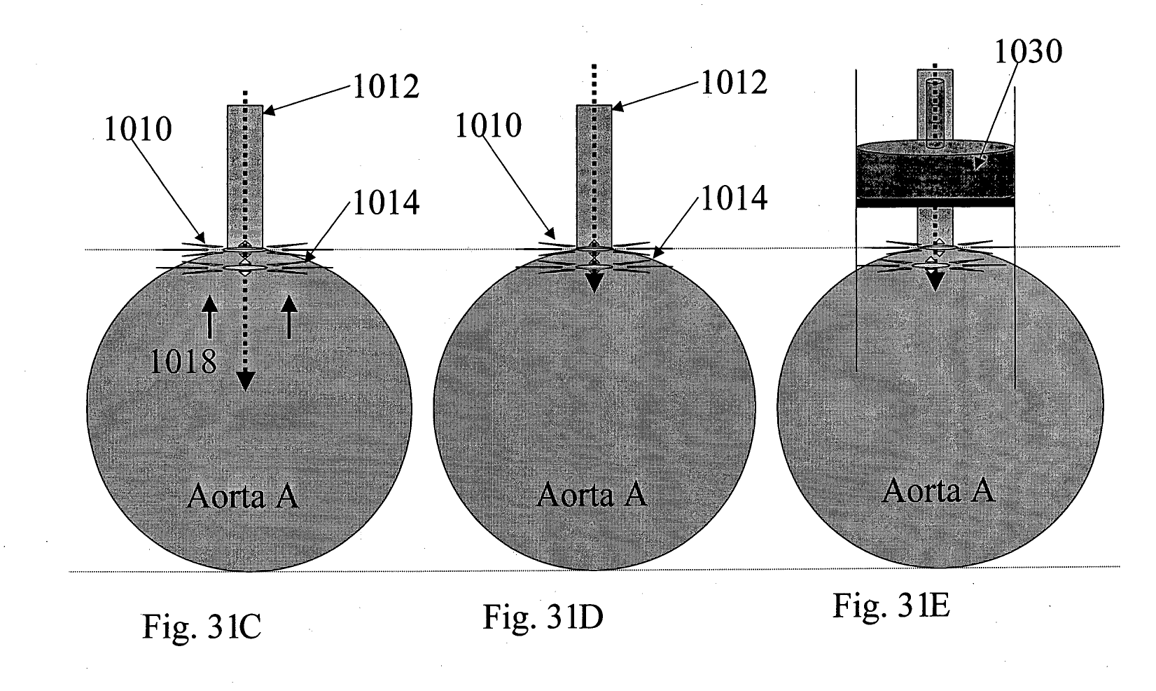

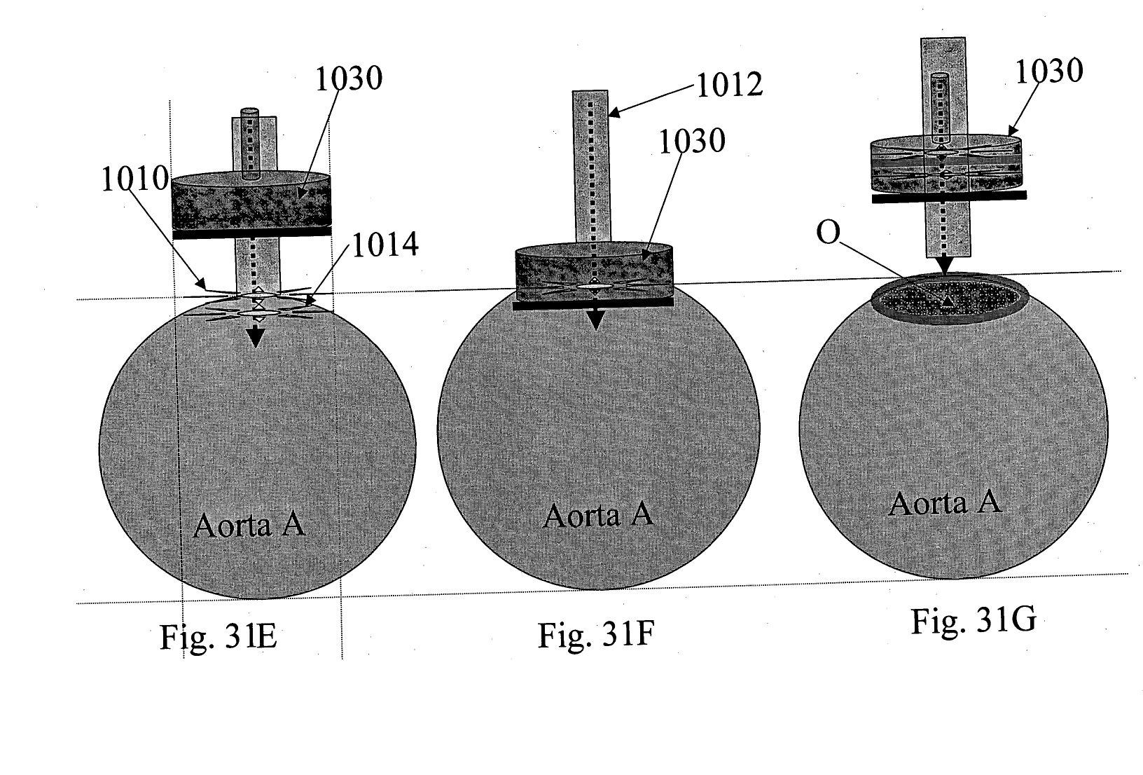

As seen in FIGS. 31A-31G, the steps of one embodiment for delivering the second portion 1014 of the tissue clamp is shown. FIG. 31A shows the portion 1014 being advanced in the hollow shaft 1012. FIG. 31B shows the portion 1014 penetrating through the wall of aorta A and into the interior as indicated by arrow 1016. FIG. 31C shows the second portion 1014 in an expanded configuration. The expansion allows the portion 1014 to engage a greater surface area of the tissue. This improves the engagement of the tissue against the first portion 1010. As mentioned, a variety of different configuration may be used for second portion 1014 including but not limited to those embodiments that expand using pneumatic techniques, electromechanical techniques, shape memory techniques, or mechanical techniques. The second portion 1014 may include a plurality of leaflets that may fold out or have struts/hinges that allow the device to expand. In one embodiment, a movable handle and/or shaft may be used to initiate expansion of the screen or portion 1014. FIG. 31C-31D shows that second portion 1014 may be drawn outward as indicated by arrows 1018 to engage the wall of the aorta A and clamp it against the first portion 1010.

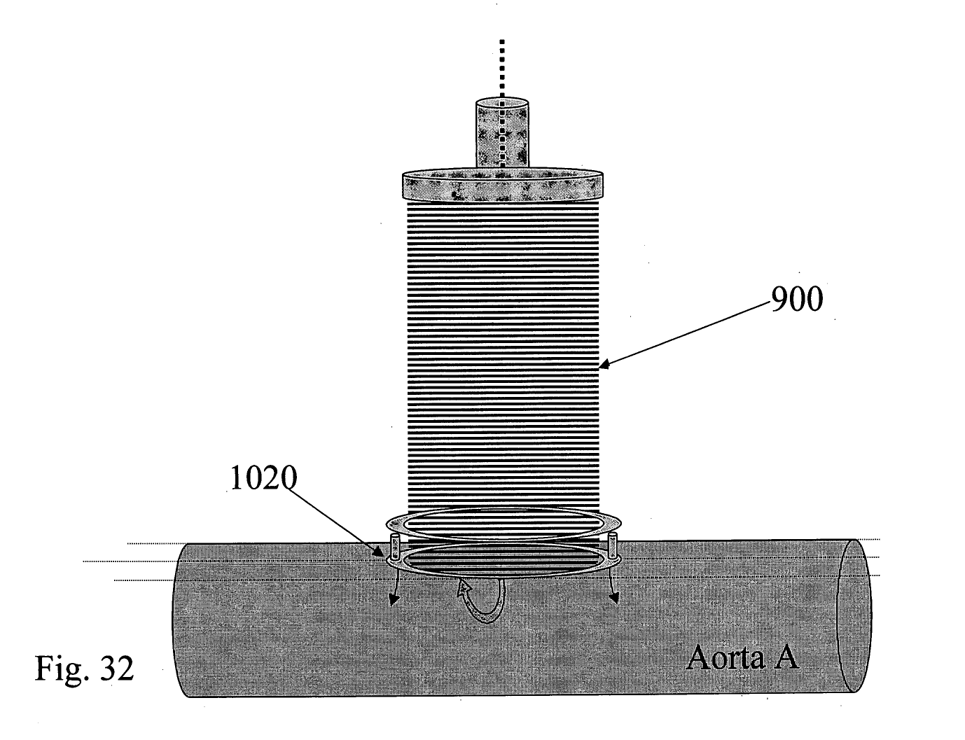

Referring now to FIGS. 31E-31G, a cutting device 1030 may be advanced to cut the tissue engaged by portions 1010 and 1014. FIG. 31E shows that this embodiment of the cutting device 1030 may be advanced coaxially along the shaft 1012 used by the second portion 1014 of the tissue clamp. It should be understood of course that other embodiments of a cutting device such as laser, ablation, or electromechanical may also be sufficient for use with the present invention. They may be arranged in circular, oval, square, rectangular, octagonal, polygonal, or other shapes/combination of shapes as desired. FIG. 31F shows the cutting device 1030 cutting through the tissue of the aorta A. The device 1030 may be designed to cut about the portion of tissue clamped between portions 1010 and 1014. FIG. 31G shows the retraction of device 1030 and the tissue clamped between the tissue clamp. The removal of tissue provides an opening O in the tissue that allows for devices such as the one-shot device described above in association with FIG. 14 to be advanced into aorta to provide treatment and/or valve replacement. This creation of an opening O is desirable in an off-pump valve replacement since it provides access to the blood vessel while a graft (as discussed in FIG. 32 below) prevents uncontrolled blood loss from the opening O.

As mentioned above, FIG. 32 shows one embodiment of graft that may be placed about the target site where the opening O will be created. The graft 900 may be used to provide access to the one-shot device of FIG. 14. As seen in FIG. 32, a seating ring 1020 may be used to secure the graft 900 to the aorta A.

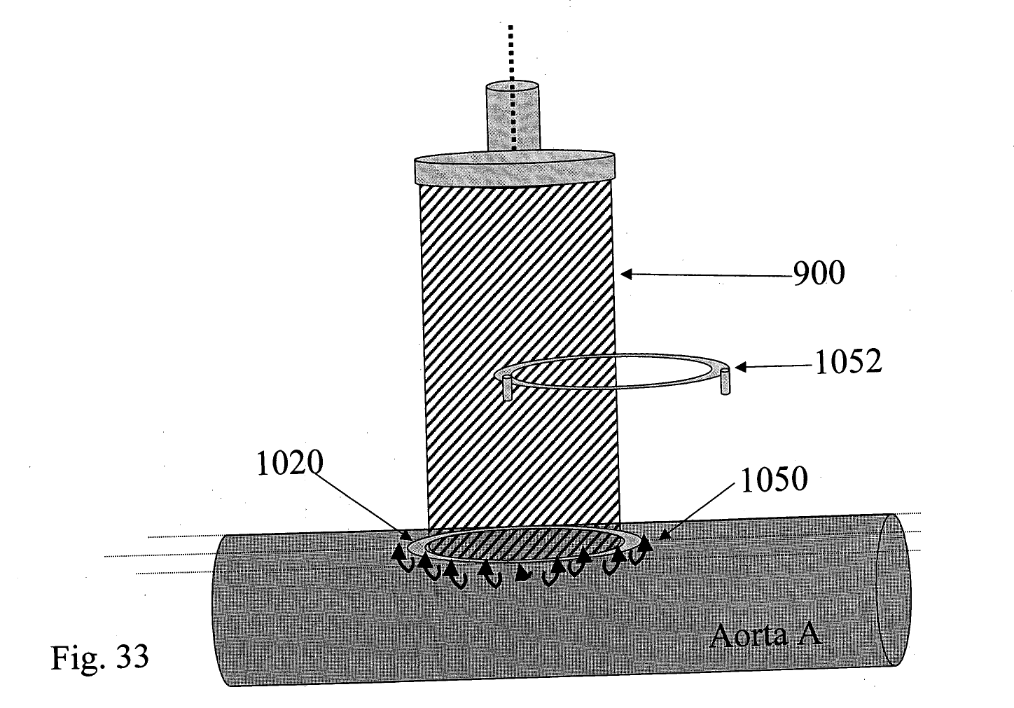

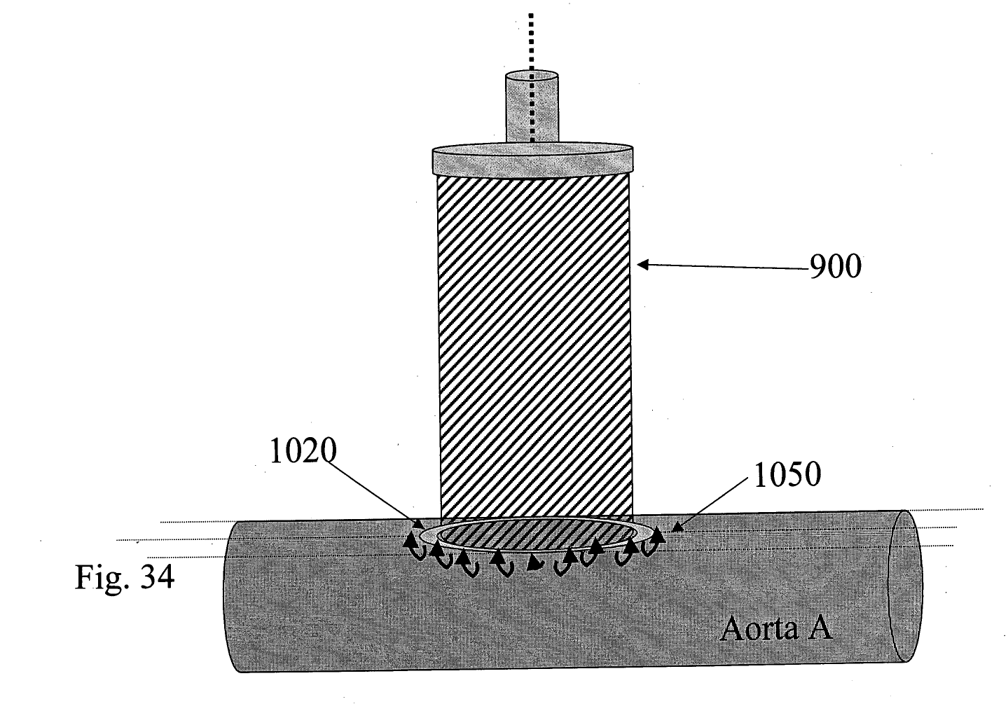

FIG. 33 shows that a plurality of fasteners 1050 may be used to secure the ring 1020 to the aorta A. The ring 1052 used to deliver the fasteners 1050 into the aorta A may be removed. FIG. 34 shows the ring 1052 removed and the fastener 1050 secured.

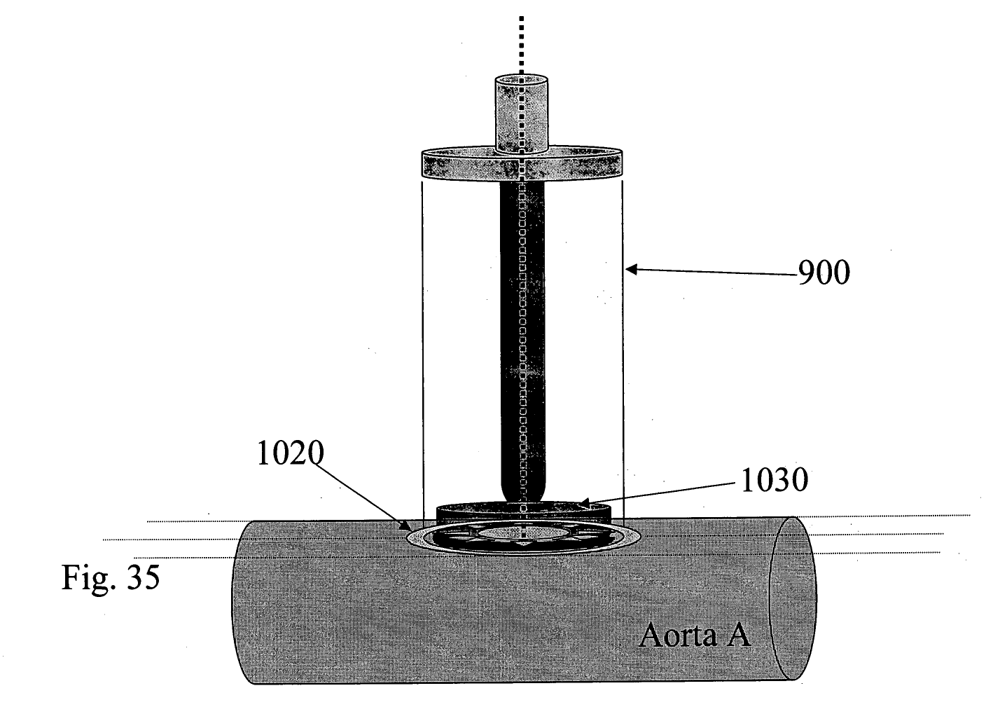

Referring now to FIGS. 35-37, various views of one embodiment of devices inside the graft 900 will now be described. FIG. 35 shows the cutting element 1030 in this cross-sectional view of the devices that may be inside the graft 900.

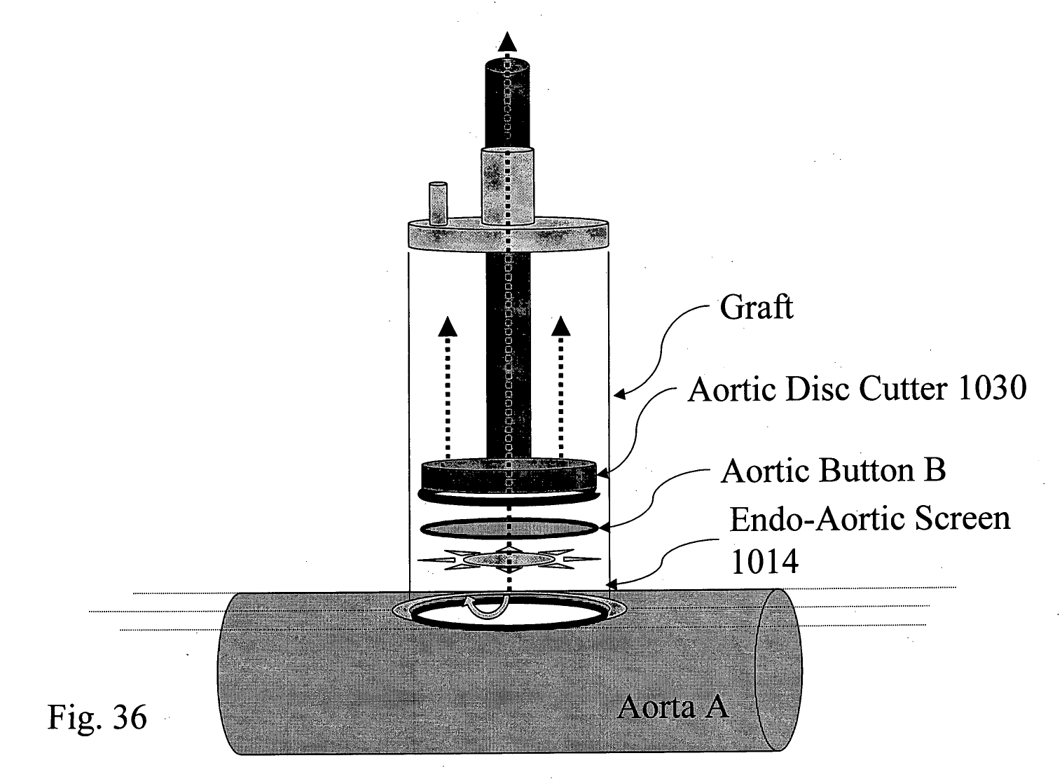

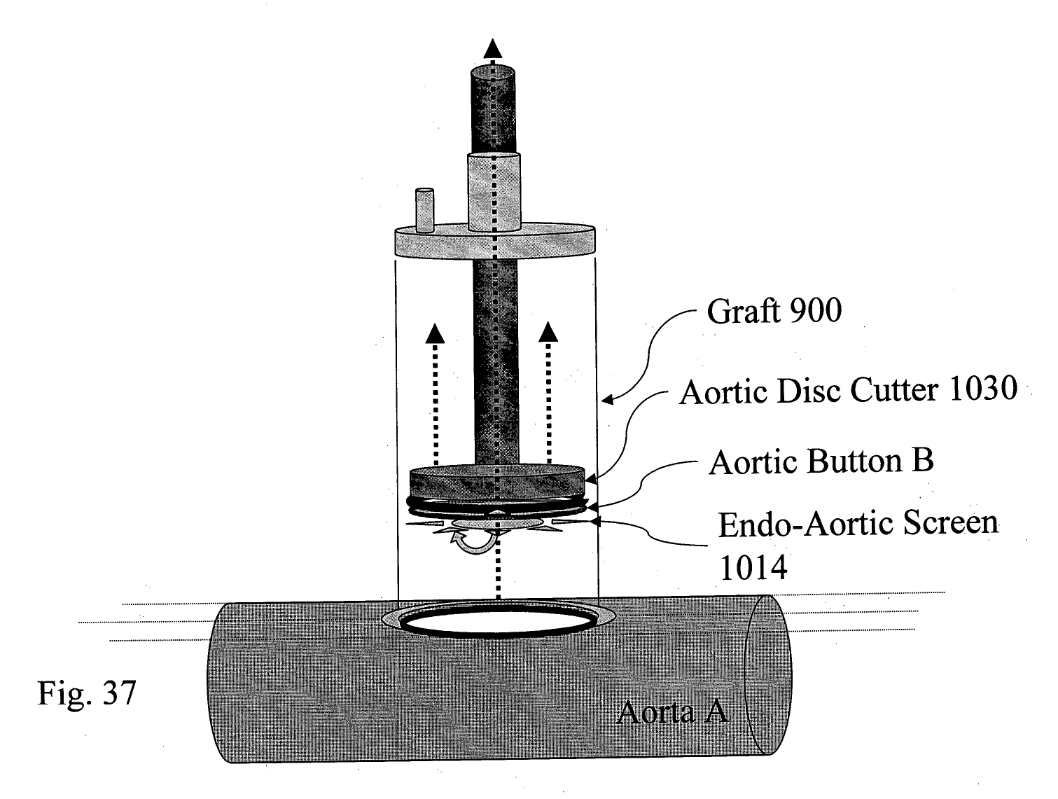

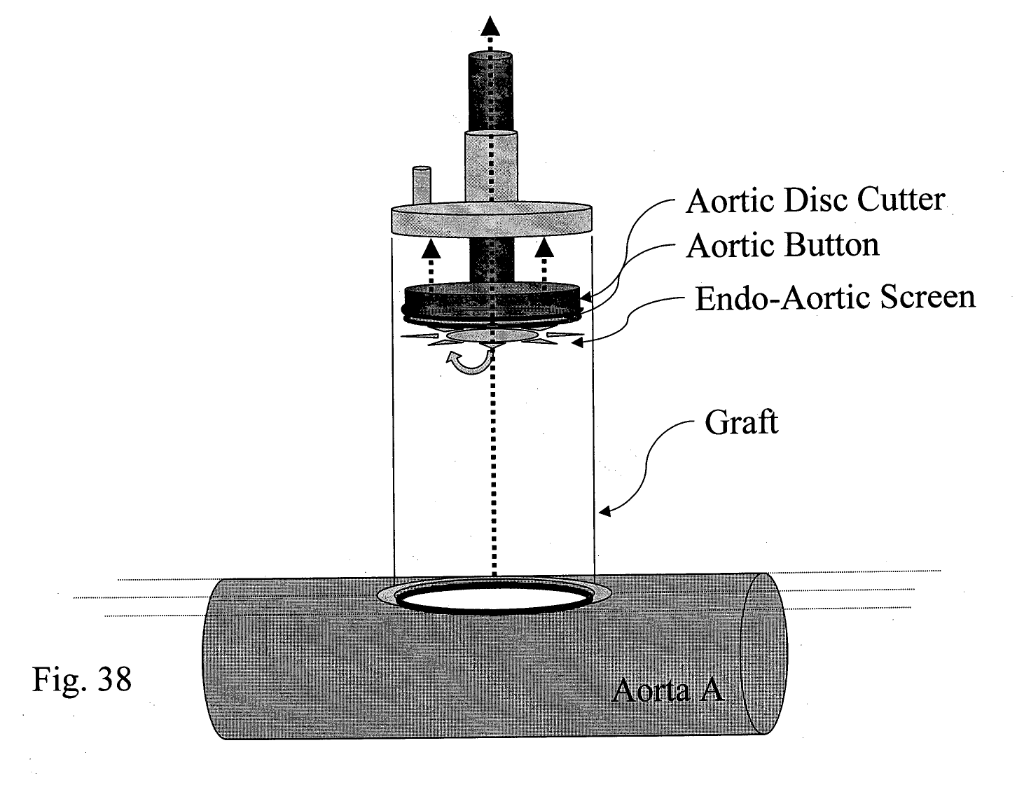

FIG. 36 shows an exploded view of the materials being removed from the aorta A. In this embodiment, the second portion 1014 may be described as a endo-aortic screen. The aortic button is the area of tissue being removed. This tissue may of course be used or reattached to the aorta A as desired. FIG. 37 shows the tissue or aortic button clamped against the aortic screen 1014. The present device may be used with an hemostatic cap on the proximal end. FIG. 38 shows a view from outside, showing a graft 900 with the hemostatic cap. After access is created, the graft may be coupled as shown in FIG. 15 above, or in some embodiments, the graft 900 also includes the one-shot device therein.

Referring now to FIG. 39, a still further embodiment of the present invention will now be described. The present invention of using a minimally invasive, off-pump technique allows for a significant reduction in the size of the entry incision used to access the aorta and insert a valve prosthesis. FIG. 39 shows the sternum S in a typically human adult. For the one embodiment of the present invention, the sternal incision 1060 may be about 3 inches long. The cut into the sternum may be about 4 inches long, which is then opened through the use of a spreader to provide access to the aorta inside the rib cage. The entire sternum S is not cut. Only an upper portion and this significantly reduces healing time and trauma to the patient.

FIG. 40 shows the aorta A and how a graft 1090 passing through the sternal incision 1060 and will eventually be attached to the aorta A via for a minimally invasive technique valve repair technique. It should be understood that graft 1090 may be just a lower portion coupled by a connector 1092 to an upper graft portion 1094. In some embodiment, the graft 1090 and 1094 are integrally formed and are not separate grafts. By way of example and not limitation, the connector 1094 may be press fit, thread to screw together, or keyed to interlock.

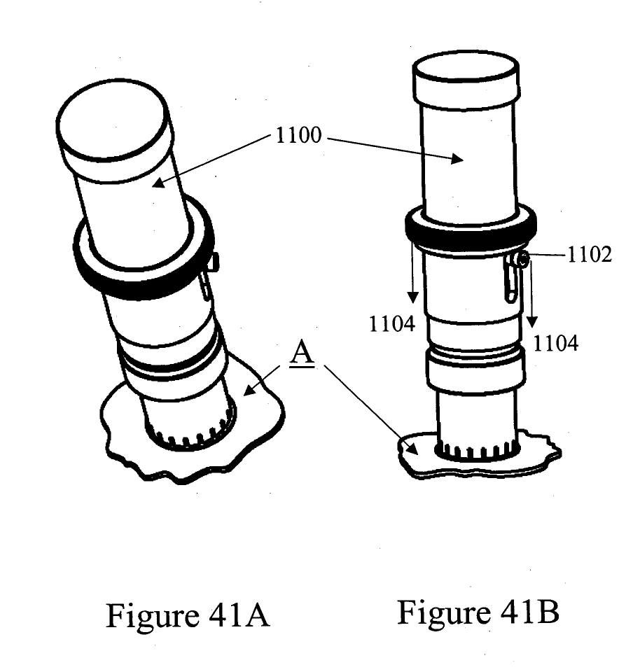

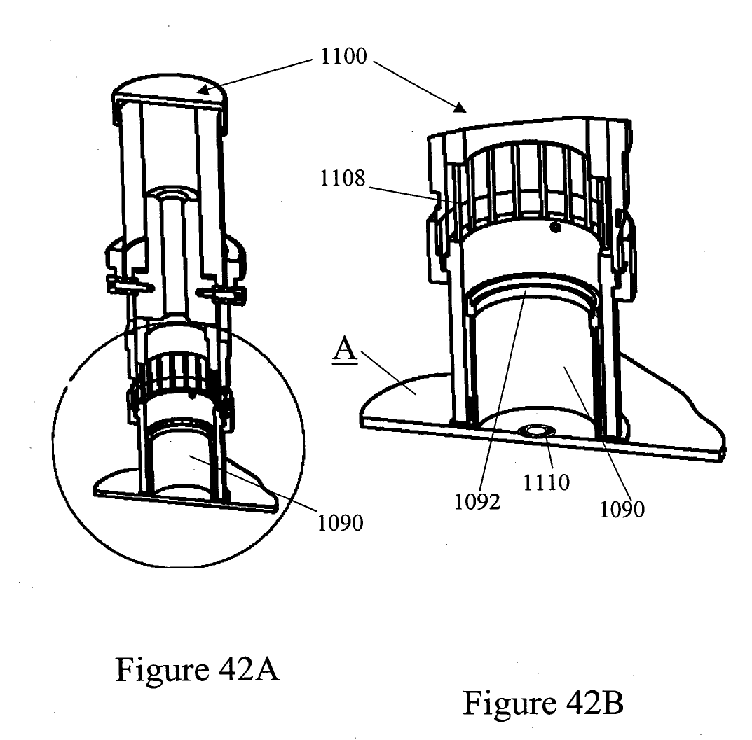

FIGS. 41A and 41B show various views of a multi-fire device 1100 for use in attaching a graft 1090 to the aorta A. The multi-fire device 1100 is similar to that described in commonly assigned, copending U.S. patent application No. ______ (40450-0008) filed Nov. 13, 2004 and fully incorporated herein by reference. The device 1100 is used to deploy a plurality of fasteners to attach the graft 1090 to the aorta A. The multi-fire device 1100 has a movable portion that drives the extensions 1102 to move downward as indicated by arrows 1104 and in so doing, move a plurality of push rods (see FIG. 42A) which ejects the fastners to attach the graft 1090 to the aorta A.

FIG. 42A shows a cross-sectional view of the device 1100. The graft 1090 is located inside the device 1100. As seen in FIG. 42B, when the push rods 1108 are moved downward, a plurality of fasteners will be ejected to attach graft 1090 to the aorta A. In some embodiments, an anchor disc 1110 may be adhered or sewn to the aorta A to help center the position of the device 1100. A guidewire (not shown) may be passed through the center of anchor disc 1110 (which may be doughnut shaped). The guidwire may be received through the center of device 1100 and guide the device into position.

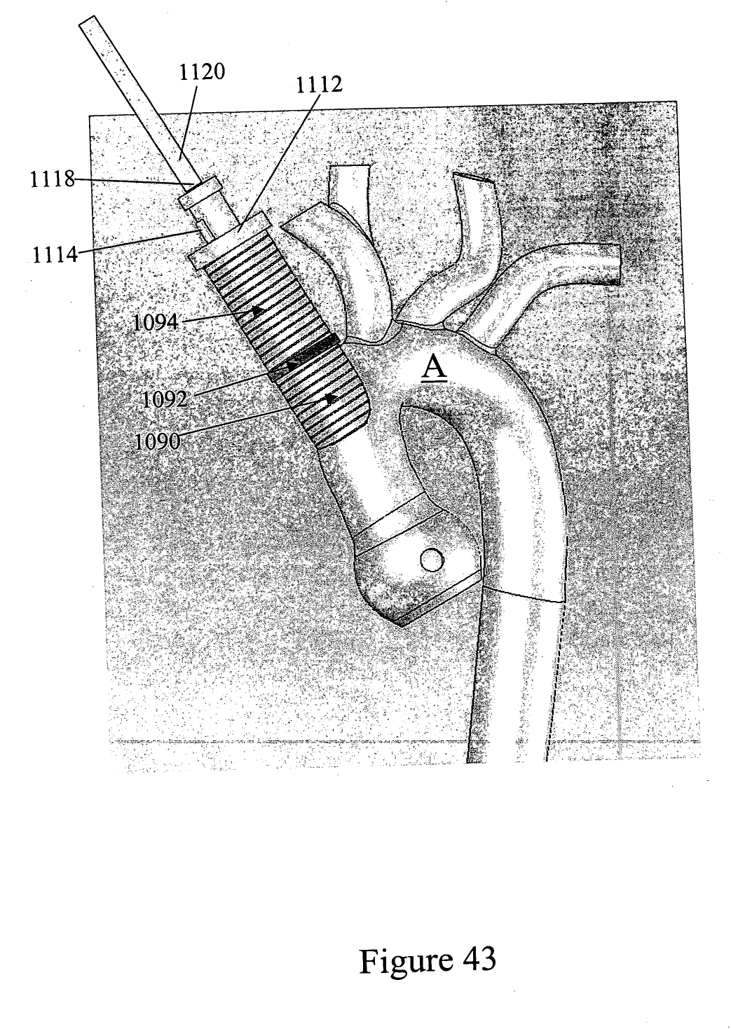

FIG. 43 shows that after graft 1090 is attached, a second graft 1090 with a cutter may be attached. The cutter is used to provide a larger opening in the aorta. The second graft 1090 may have a hemostatic cap 1112 with an air evacuation port 1114 at the proximal end of the graft 1090. The port 1114 may be used to bleed off any air that may remain in the graft 912. An opening 1118 may be included to allow a shaft or other extension to extend outward from the graft. The shaft of the cutter 1120 may be sized to maintain a seal with the opening 1118. An o-ring may also be used to maintain a seal with the shaft of the cutter 1120.

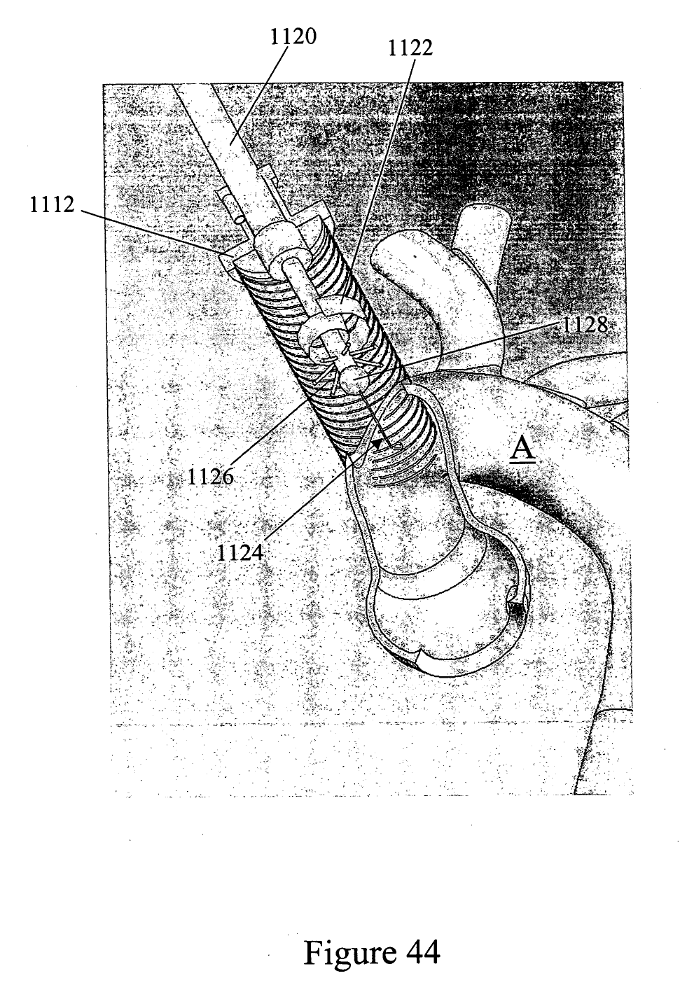

FIG. 44 shows a cross-sectional view of graft 1090 and 1092 with the cutter 1120 inside. In this present embodiment, the cutter 1120 has a spiral blade 1122. Other embodiments may have a round blade, a pizza cutter type blade configuration, a punch, or the like. In this embodiment, a guide wire 1124 has already penetrated through the wall of the aorta A. The guide wire 1124 will be used to guide the spiral blade 1122 to cut at the desired location. The supports 1126 are shown extended although they are typically in a retracted position prior to passing through the wall of the aorta A. The supports 1126 will act as an anvil against which the blade 1122 can exert cutting force.

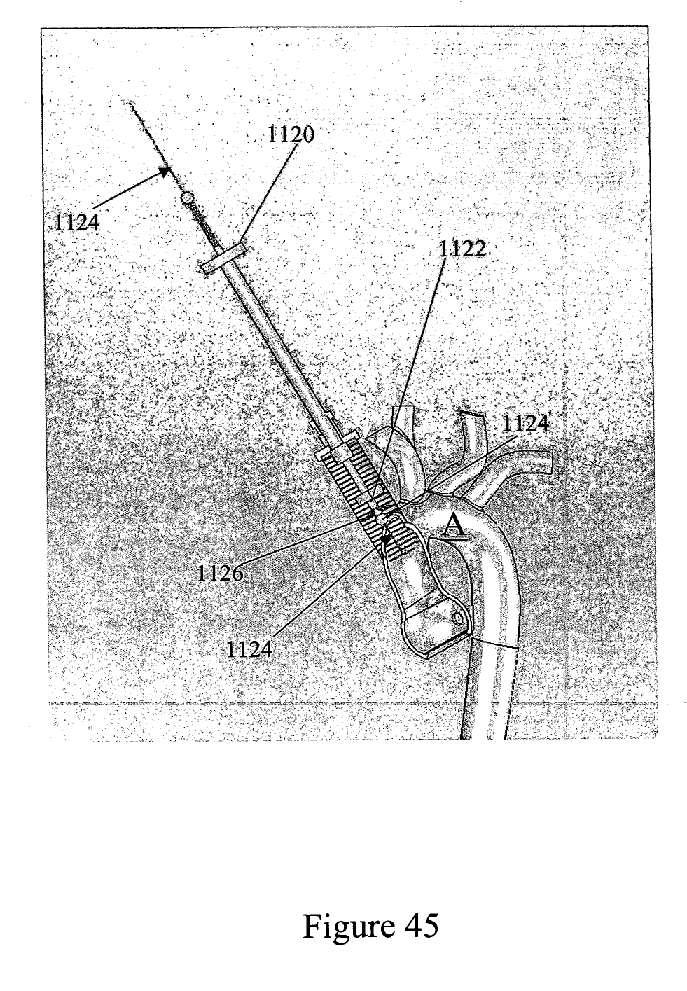

FIG. 45 shows the entire cutter device 1120 and how it is positioned inside the graft 1090 and that a proximal portion extends outward from the graft. The cutter 1120 will be moved forward towards the aorta A so that the distal ball portion 1128 will pass through the opening where the guide wire 1124 has pass through the aorta.

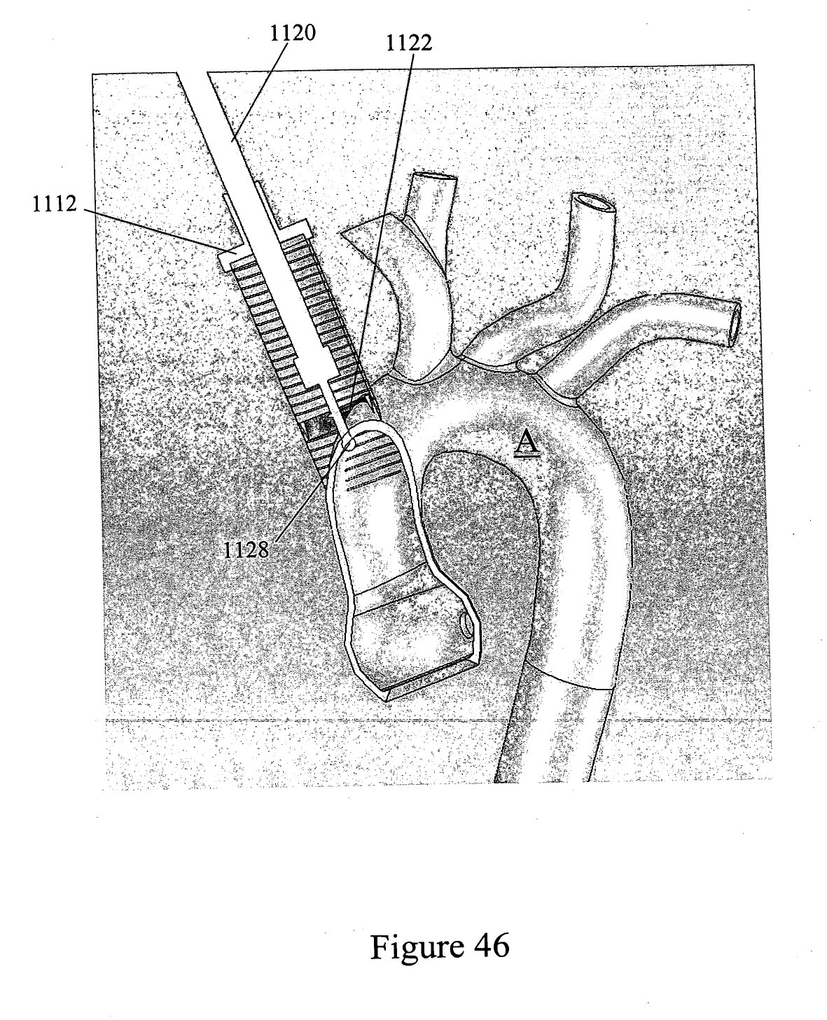

FIG. 46 shows how the cutter 1120 may use the blades 1122 to cut into the aorta to remove an area of the aortic wall. This area to be removed may be referred to as the aortic button B. The rotation of the cutter 1120 will allow the blades 1122 to cut into the tissue. The removal of a portion of the aortic wall will provide access for other devices into the aorta and into the heart.

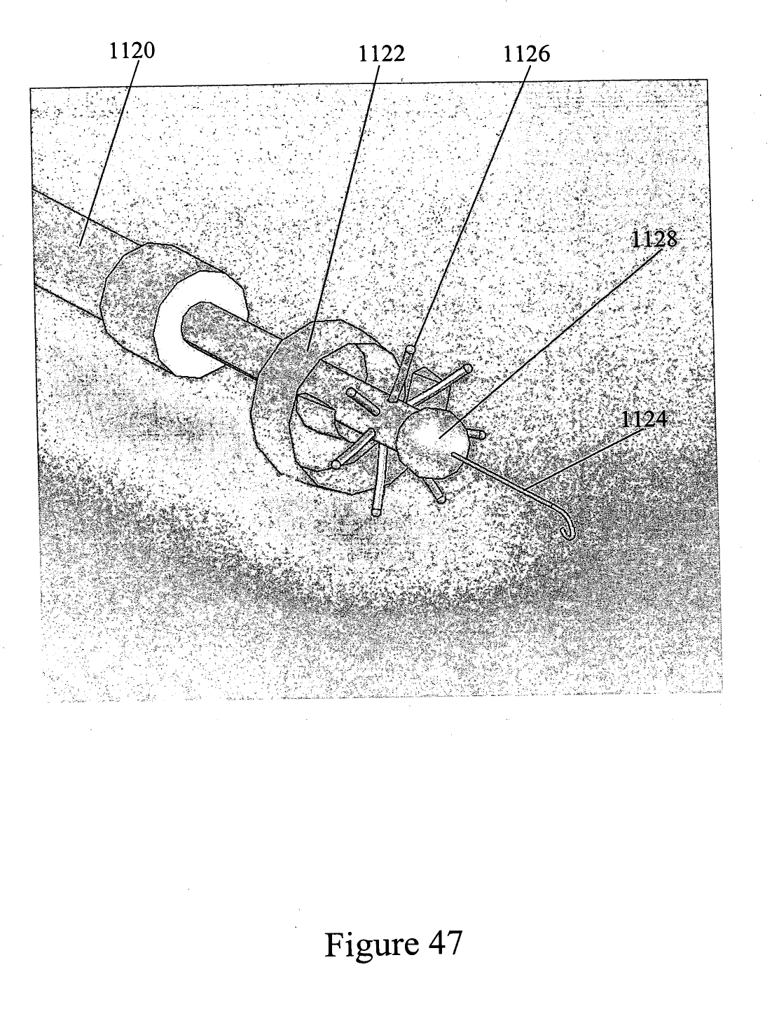

FIG. 47 shows a close-up perspective view of a distal end of the cutter 1120. The cutting blade 1120 is shown with the supports 1126 extended. In one embodiment, the tissue to be cut will be between these parts. The guide wire 1124 and the rounded front end 1128 are also clearly shown.

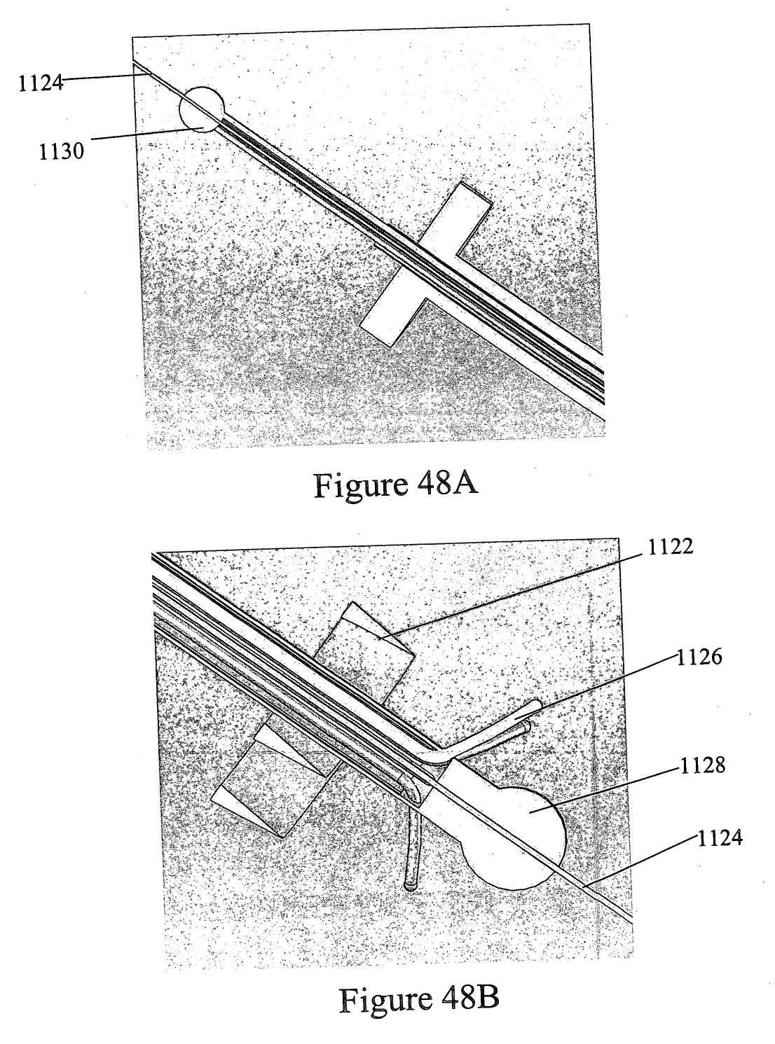

FIGS. 48A and 48B show cross-sectional views of the proximal and distal end of the cutter 1120. FIG. 48A shows that at the distal end of the cutter 1120, a shaft portion 1130 with a rounded end is movable and that the guide wire 1124 extends all the way through the cutter 1120. In the present embodiment, moving the shaft portion 1130 forward will extend the supports 1126 as shown in FIG. 48B. In one embodiment, the supports 1126 are shape memory device that will assume the bent configuration when they are extended outward from the device 1120.

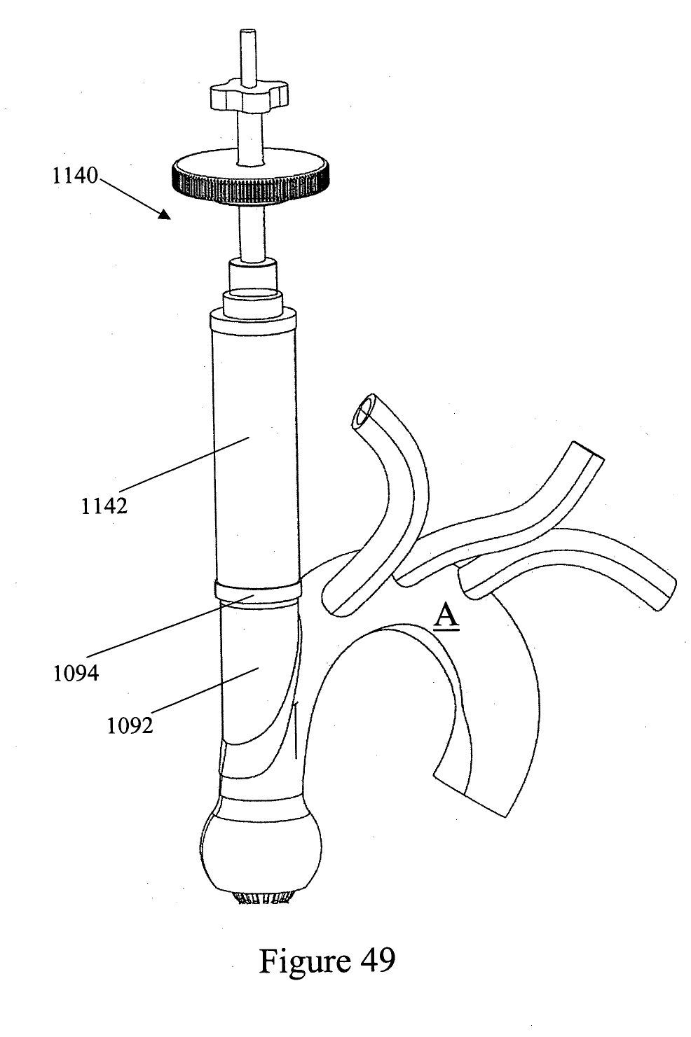

FIG. 49 shows that after the aortic button is removed as described above or in a manner similar to that shown in FIGS. 29-38, a multi-fire device 1140 with a valve prosthesis may be attached to the graft 1090. Prior to attaching the device 1140, the cutter 1120 and its associated graft 1090 may be removed. The cutter 1120 is withdrawn proximally and the graft 1090 below the connector 1094 is clamped to prevent the loss of blood during the exchange. The graft 1090 with the cutter 1120 is disconnected and a graft 1142 with the device 1140 is attached.

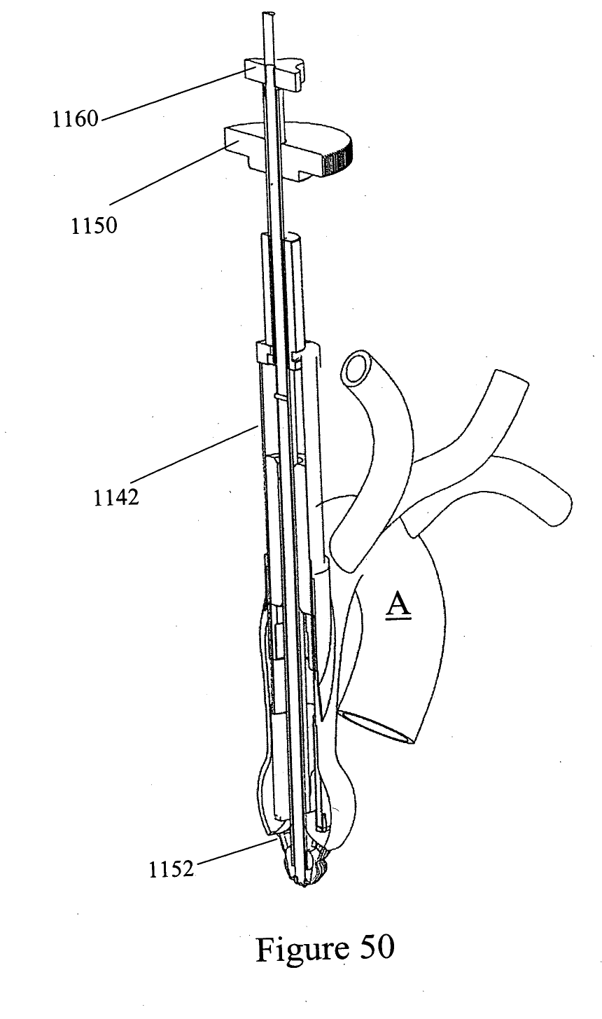

FIG. 50 shows a cross-sectional view of the device 1140 in position in the aorta A. The multi-fire device 1140 has a longer distal portion, but otherwise operates in a manner substantially similar to the device described in copending U.S. application Ser. No. ______ (Attorney Docket No. 40450-0008) fully incorporated herein by reference. When handle 1150 is retracted, the anvil 1152 will deploy to engage valve tissue. The forward or downward movement of handle 1160 will then move push rods that will deploy the fasteners to attach the valve prosthesis.

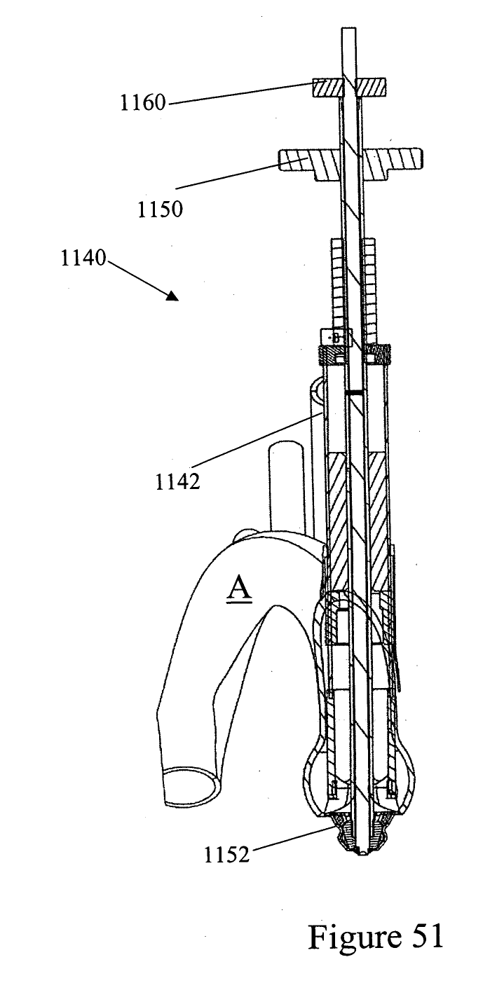

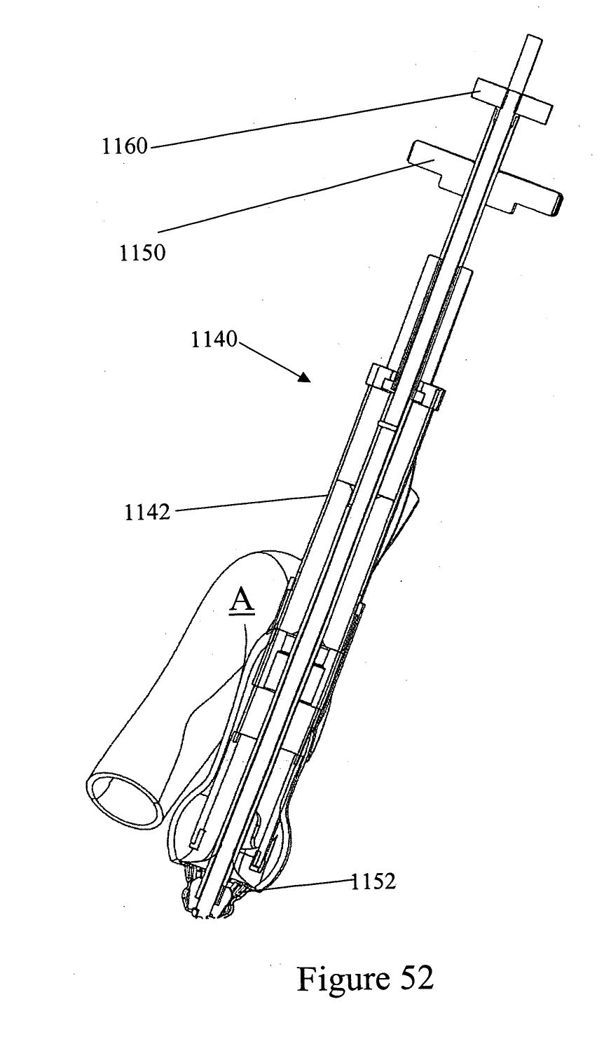

FIGS. 51 and 52 provide other sectional view of the device 1140. It should be understood that in some embodiments, the method may involve attaching a graft with a valve leaflet cutter and inserting a valve leaflet cutter to remove the valve leaflets prior to attaching the graft with the device 1140. Other embodiments may leave the existing leaflets in place and deploy the valve prosthetic with the original valve still there.

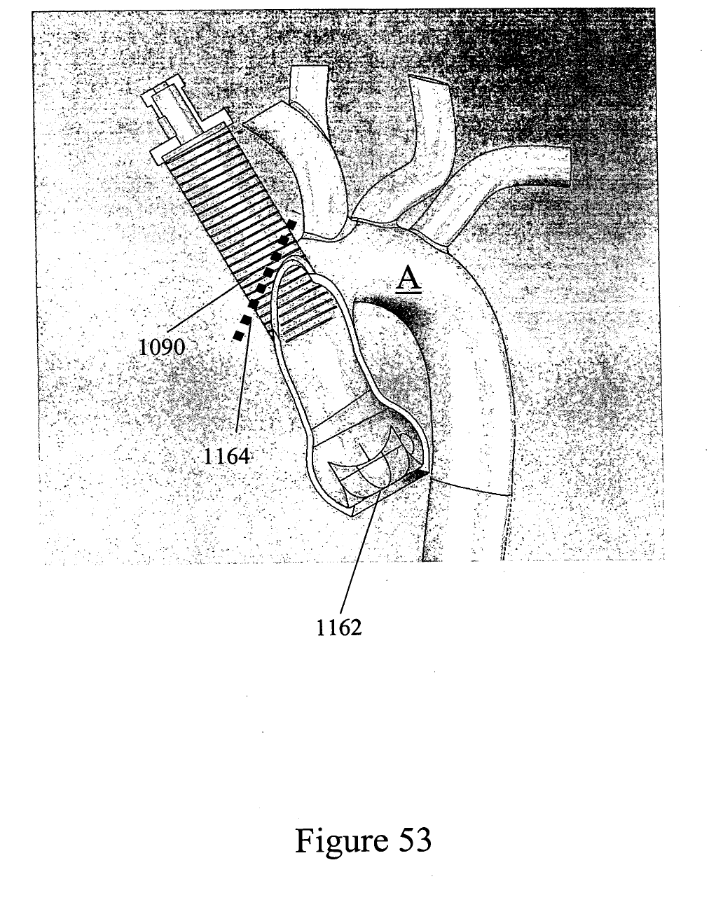

FIG. 53 shows the operation nearly completed. A valve prosthesis 1162 is shown in position. The graft 1090 may then be clamped off and then cut as indicated at line 1162. This will then be sealed to prevent blood loss through the area where the aortic button was removed. A portion of graft 1090 remains in place after the procedure is completed.

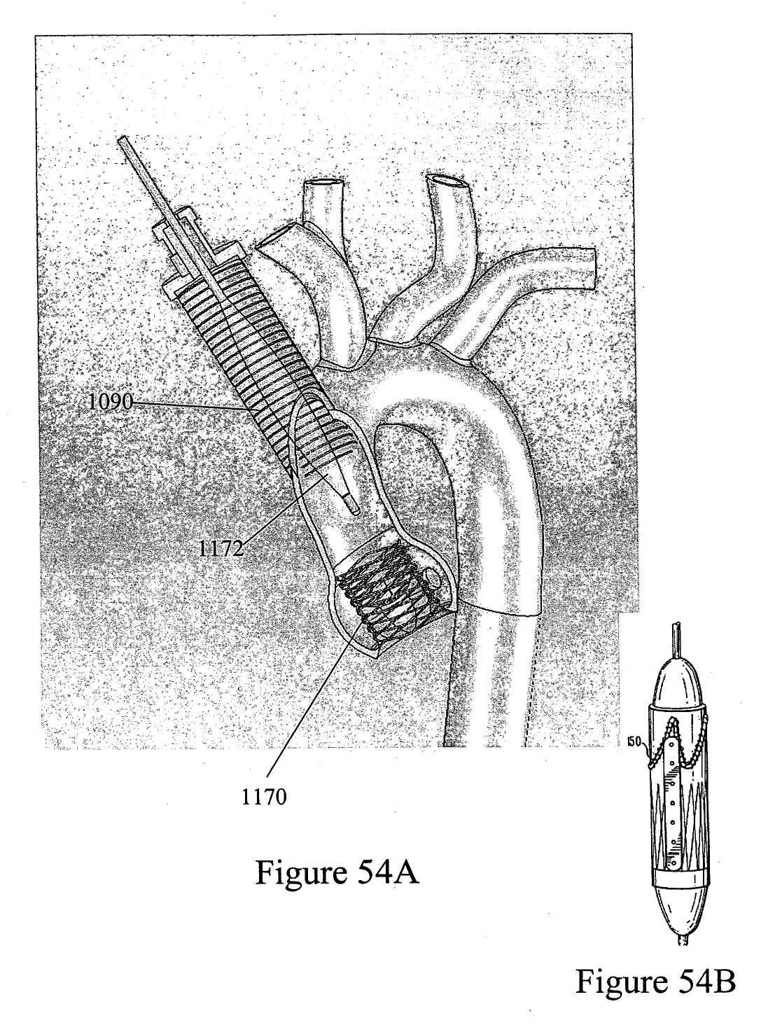

FIG. 54A shows that the present invention may be adapted for use with any type of valve delivery device. In FIG. 54A, an expandable valve prosthesis 1170 is shown to have been delivered in place by an expandable delivery device 1172. The graft 1090 again provides the blood containment capability to allow for off-pump procedures. the delivery device 1172 may be a balloon device that is expanded to expand prosthesis 1170. Other embodiments may use self expanding valves. FIG. 54B shows how the expandable valve may be mounted onto a delivery device. The device comprises a support stent, comprised of a deployable construction adapted to be initially crimped in a narrow configuration suitable for catheterization through the body duct to a target location and adapted to be deployed by exerting substantially radial forces from within by means of a deployment device to a deployed state in the target location, and a valve assembly comprising a flexible conduit having an inlet end and an outlet, made of pliant material attached to the support beams providing collapsible slack portions of the conduit at the outlet. The support stent is provided with a plurality of longitudinally rigid support beams of fixed length. When flow is allowed to pass through the valve prosthesis device from the inlet to the outlet, the valve assembly is kept in an open position, whereas a reverse flow is prevented as the collapsible slack portions of the valve assembly collapse inwardly providing blockage to the reverse flow.

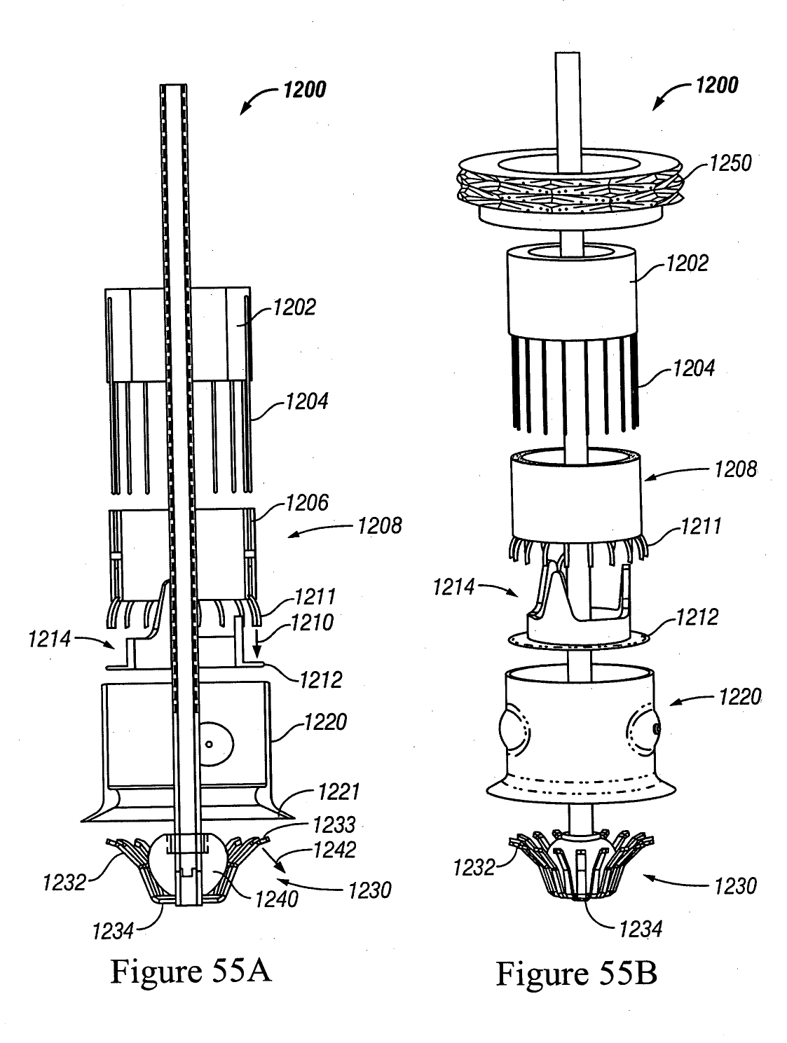

Referring now to FIGS. 55A and 55B, another embodiment of the present invention will now be described. FIG. 55A is a cross-sectional view of one embodiment of a delivery device 1200 according to the present invention. The device 1200 includes a plunger 1202 having a plurality of pushing elements 1204. These pushing elements 1204 will pass through passageways 1206 in the fastener housing 1208 to push the fasteners in the passageways 1206 outward in the direction indicated by 1210. The fasteners will then pass through a sewing ring 1212 of the prosthetic valve 1214. The prosthetic valve 1214 may be pre-loaded and positioned inside the blood vessel 1220 having the target tissue area. For the device of FIG. 55A, the valve prosthetic may be mounted along the inside surface of the fastener housing 1208. By way of example and not limitation, the fastener housing 1208 may have a circular, oval, polygonal, or other cross-sectional shape.

In one embodiment, the fastener housing 1208 may be advanced forward by a plunger or by user actuation to advance the sharpened guide tube 1211 to pierce the sewing ring 1212. After the tube 1211 pierces the sewing ring, the fastener may then be deployed. Some embodiments may actuate the fasteners without having the guide tubes 1211 penetrate the sewing ring. The use of a plunger will simultaneously eject a plurality of fasteners from the guide tubes 1211.

As seen in FIG. 55A, the delivery device 1200 may be used with another embodiment of the tissue engagement device 1230 which is made to expand and engage the tissue at 1221. A cut-out section of aortic valve tissue 1220 is drawn to show its relationship to the position of the tissue engagement device 1230. In the present embodiment, the tissue engagement device 1230 may have a plurality of fingers 1232 that act as support elements. These fingers 1232 are coupled to a central disc 1234. FIG. 55A shows the tissue engagement device 1230 in an expanded configuration. A shaped plunger member 1240 is inserted into the center of the plurality of fingers 1232 and the shaped plunger member 1240 has a circumference sufficient to deflect the fingers 1232 to a position where the fingers are pushed radially outward as indicated by arrow 1242. By way of example and not limitation, the shaped plunger member 1240 may be rounded as shown in FIG. 55A or it may be, but is not limited to, shapes such as spheres, cones, wedges, cubes, polygons, or any single or multiple combination of the above. As seen in this embodiment, the tissue engagement device 1230 is expanded by drawing the fingers 1232 around the ball or pushing the ball into the tissue engagement device 1230. Although not limited to the following, the fingers 1232 may be made from nickel titanium alloy, stainless steel or polymer. In other embodiments, the tissue engagement device 1230 may have a hinge configuration with parts that may be articulated to expand.

Hinged fingers when in its undeployed position will remain at its minimum radial position to allow passage through the prosthetic valve opening once the tissue engagement device is passed through the valve or the aorta. The articulating hinged fingers can then be deployed to a larger radial configuration to support the tissue at point 1221. In some embodiments, the expandable device will contact the device to hold it in position. The device may include a support surface 1233 to contact the tissue. In some embodiments, the support surface 1233 may be used to align or stop the fastener housing.

In some embodiments, the fingers 1232 may be coupled together by a mesh material such a DARON™, Dacron™, a firm rubber substance, GORTEX™, any combination of the above, or similar substances to capture debris that may be created by the valve repair procedure. In some embodiments, the fasteners will align to extend outward in the gaps between fingers 1232 so that the fingers do not interfere with deployment of the fasteners.

FIG. 55B shows an exploded perspective view of the embodiment of FIG. 55A. The FIG. 55B also shows that a handle 1250 may be included to facilitate the pushing of plunger 1202 to eject the fasteners and attach the prosthesis 1214 to target tissue. FIG. 55B shows the prosthetic valve 1214 on the inside of the fastener housing 1208. In this embodiment of the delivery device 1200, the fasteners will embed through the shoulder or sewing ring 1212 of the valve 1214.

As seen in FIG. 55B, the needles may pass through a straight portion when it exits. In such a configuration, it may be desirable to key the passageway and the cross-section of the fastener so that the fasteners will extend outward and curve in the desired direction. The present embodiment passes through the top of the shoulders or sewing rings and then hooks.

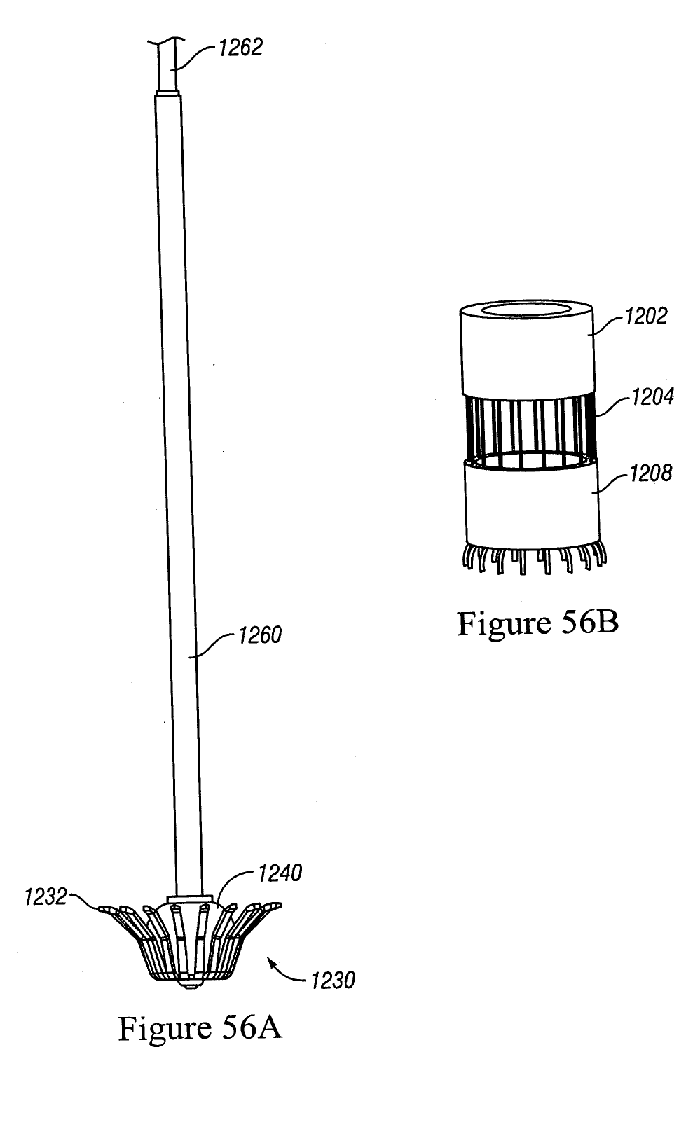

Referring now to FIG. 56A, one embodiment of the tissue engagement device 1230 is shown. In this embodiment, the shaped plunger member 1240 may be coupled to a shaft 1260. The shaft 1260 may be fixed along the longitudinal axis of the device 1200. In other embodiments, the shaft 1260 may be slidably mounted within the device 1200. The shaft 1260 may be slidably mounted over another shaft 1262 which is coupled to the tissue engagement device 1230. This allows the device 1230 to traverse. The shaped plunger member 1240 and the device 1230 may both translate or move relative to each other. This telescoping configuration allows the ball-shaped plunger member 1240 to be moved inside the tissue engagement device 1230 to expand the fingers 1232 outward. Other embodiments may have the shaft 1260 coupled to the device 1230 and the shaped plunger member 1240 coupled to shaft 1262.

Referring now to FIG. 56B, the plunger 1202 is shown with the fastener pushers 1204 engaging the fastener housing 1208. The fasteners are held inside the housing 1208 prior to being deployed for use. In one embodiment, the fasteners are made of pre-shaped superelastic nitinol material which is held in place within the fastener housing due to friction force exerted by the pre-shaped material.

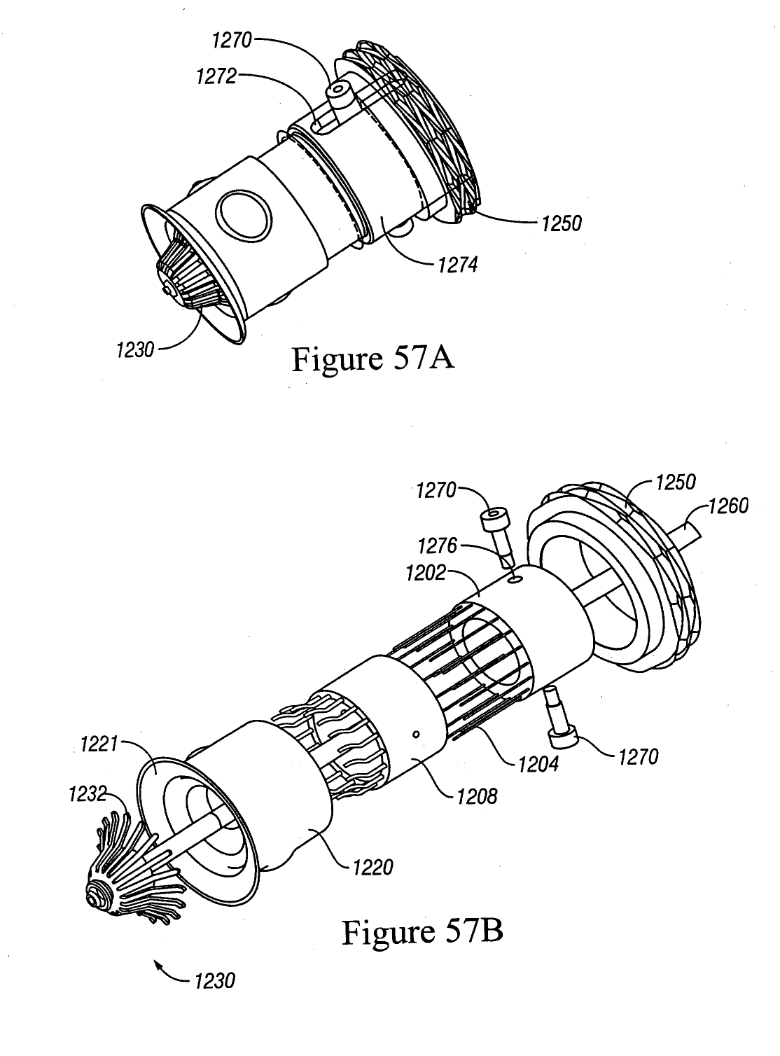

Referring now to FIGS. 57A and 57B, perspective view of the device 1200 are shown. FIG. 57A shows the device 1200 fully assembled and in a configuration where the plunger 1200 has been advanced towards a distal end of the device 1200 to deploy the fasteners. As seen in FIG. 57A, the handle 1250 may be used to push on pins 1270 to advance the plunger 1202. The pins 1270 may travel down a straight groove 1272 formed on an outer housing 1274. FIG. 57A also shows that for the present embodiment, the tissue engagement device 1230 may be sized to be deliverable into the blood vessel 1220.

FIG. 57B shows an exploded perspective view where the pins 1270 are shown to engage the plunger 1202 via holes 1276 formed in the plunger. In this view, the prosthetic valve is inside the cut-out aortic section, which is supported from the bottom with the tissue engagement device 1230 at location 1221 when the fasteners are deployed to engage the prosthetic valve into the aortic tissue 1220.

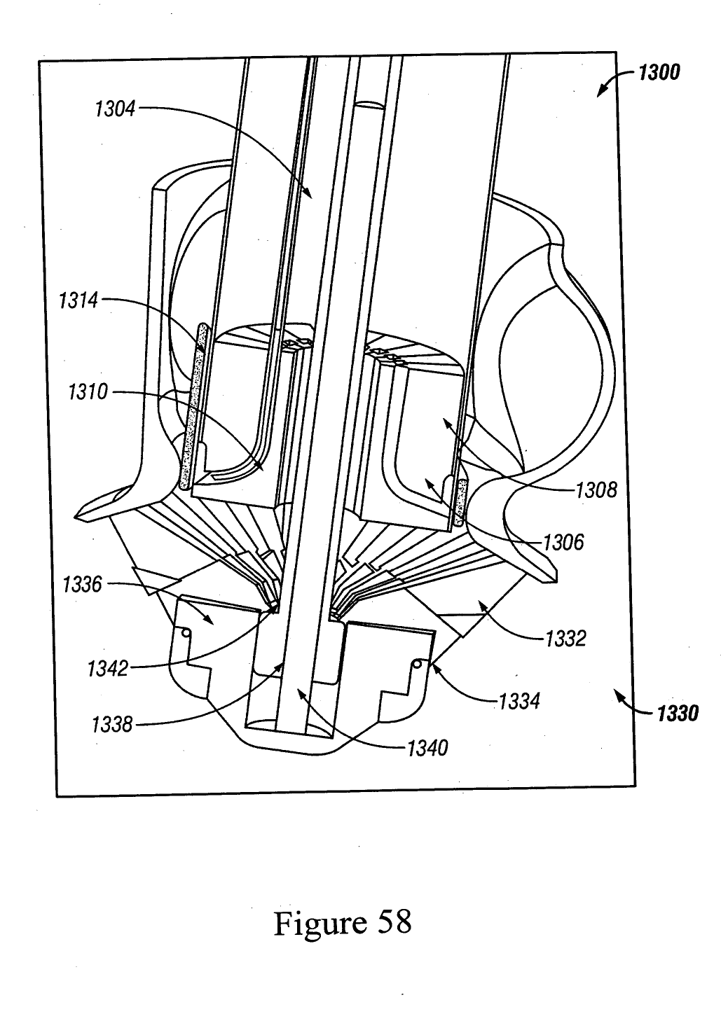

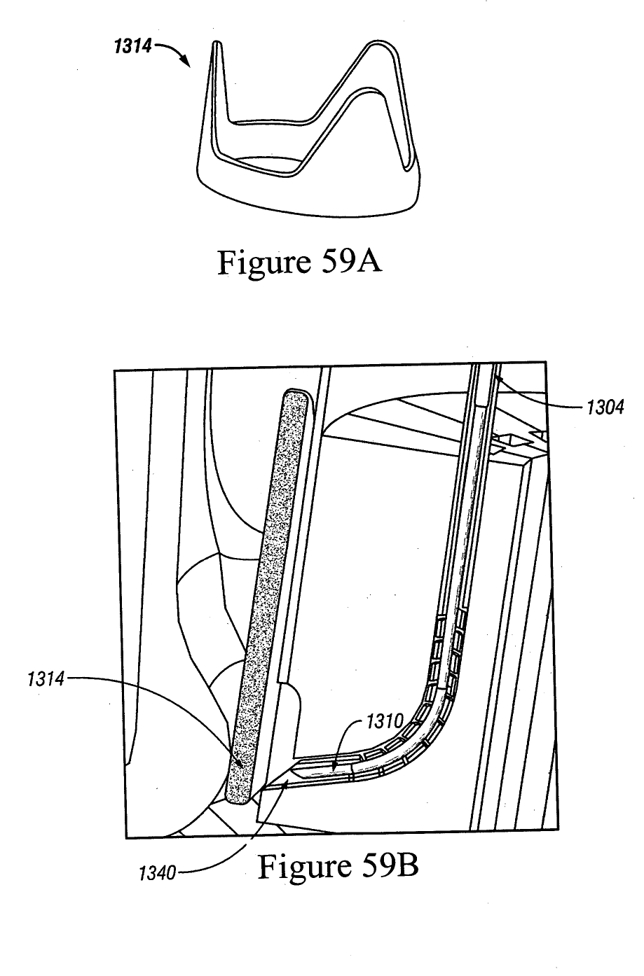

Referring now to FIG. 58, yet another embodiment of the present invention will now be described. FIG. 58 shows a cross-section view of a prosthetic delivery device 1300. The device 1300 may have a fastener housing 1308 with passageways 1306 for guiding the fastener 1310 in a desired direction. In this particular embodiment, the valve 1314 is mounted about the fastener housing 1308. As will be described in more detail in FIG. 59A, the fasteners 1310 will pass through the valve and then into the target tissue.

This embodiment uses a support device 1330 having a plurality of hinged fingers 1332 attached at a hinge point 1334 to a base 1336. A slider 1338 is moveable relative to base 1336 and is slidably mounted over the shaft 1340. The slider 1338 may be moved to engage an edge 1342 of the finger 1332 to urge the finger to a position that expands the device 1330. The fingers 1332 may be biased to retract as indicated by arrow 1334 to its original position to configure the device 1330 in a collapsed configuration. The fingers have may have a support surface near the distal end of each finger to facilitate contact with tissue and/or the prosthesis.

FIG. 59A shows a perspective view of a valve 1314 that does not include a sewing ring. The valve 1314 will be slidably mounted about the housing 1308.

FIG. 59B shows an enlarged cross-section view of the embodiment of device 1330 from FIG. 58. The fastener 1310 and push rod 1304 are more clearly shown. As seen in FIG. 59B, the fastener 1310 and push rod 1304 are actually housed inside a hollow piercing member 1340. The hollow piercing member 1340 may act as a guide tube and have a portion near the sharpened tip that is configured to be easily bendable. By way of example and not limitation, portions can be removed from the member 1340 to facilitate bending. The hollow piercing member 1340 may also be made from two pieces, which may then be integrated together. This allows for a more expensive sharpened tip portion coupled to a less expensive tube portion which can extend proximally to a plunger or other driver for actuation. There can be a mechanical stop to limit the travel of the plunger which actuates the member 1340. In some embodiments, a travel or 3-4 mm is sufficient for piercing through the valve prosthesis and into the tissue.

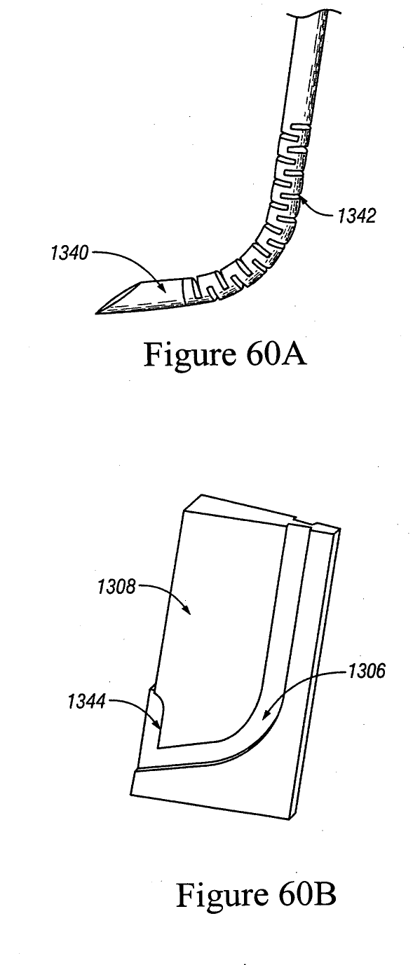

As seen more clearly in FIG. 60A, the hollow piercing member 1340 may be configured to curve within the passageway 1306 by having a plurality of cut-outs 1342 along the portion of the hollow piercing member 1340 that will curve with the passageway.

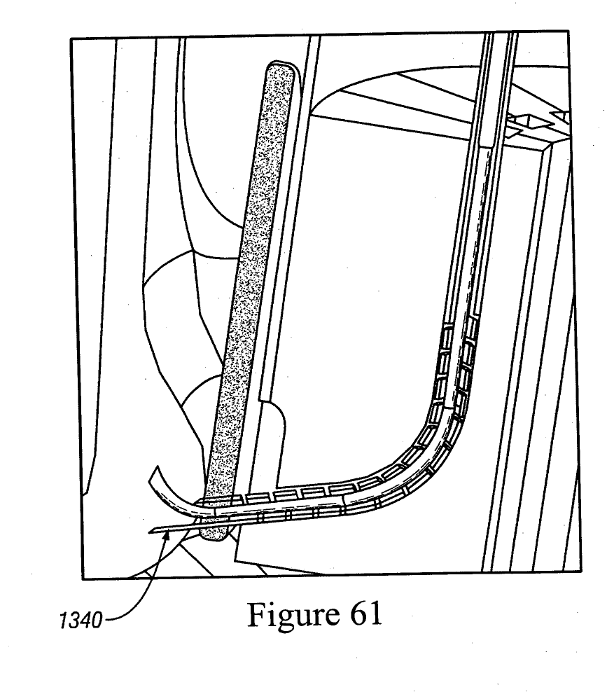

FIG. 60B shows how passageway 1306 is curved to guide the hollow piercing member 1340 and the fastener 1310. The fastener housing 1308 may include a cavity area near the exit of the passageway 1306. As will be seen more clearly in FIG. 61, this provides clearance for the fastener to pass through the valve material at one location and loop back through the valve at a second location.

Referring now to FIG. 61, one method of deploying a fastener 1310 will now be described. As seen in FIG. 61, the hollow piercing member 1340 is extended outward from the passageway 1306. By way of example and not limitation, the member 1340 may extend a distance of about 3 mm. In the present method, the member 1340 will piercing through the valve 1314 and into the target tissue. Once the member 1340 has reached a desired penetration depth, the fastener 1310 is then deployed. The hollow guide member 1340 guides the member through the valve 1314 and prevents fastener 1310 from curving too early. This allows the fastener 1310 to penetrate more deeply into the target tissue and provide a more secure anchor. As seen in FIG. 61, the fastener 1310 is beginning to curve and point back towards the valve 1314.

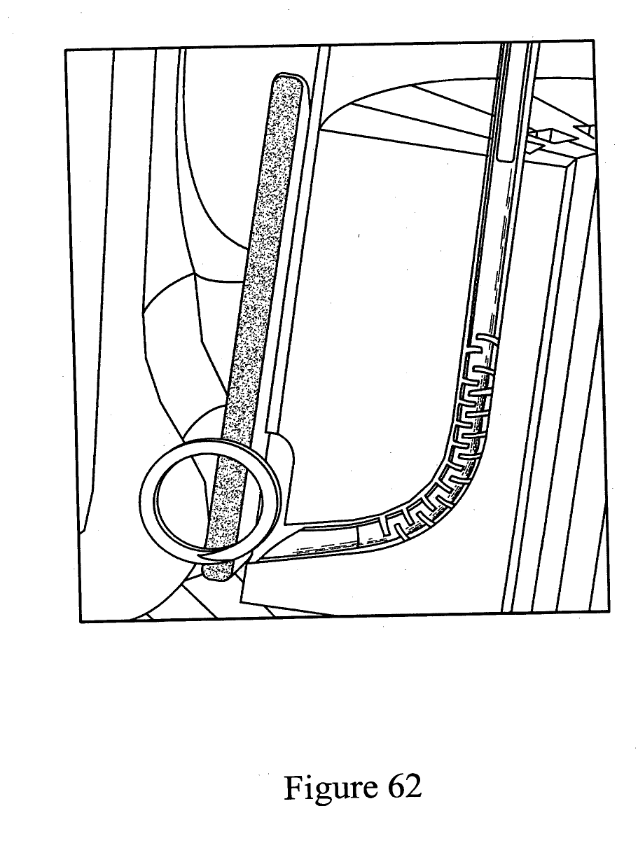

Referring now to FIG. 62, the fastener 1310 is shown in a curved configuration. The fastener 1310 is shown to have formed two loops, passing through the valve material four times. The cavity 1344 allows for the loops to be formed without interference from the housing 1308. The FIG. 62 also shows the fastener piercing the valve at two different locations as it loops through the valve prosthetic. Some embodiments may pierce at more than two different locations, depending on how many loops are formed and where the fastener reenters the valve prosthetic.

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. For example, with any of the above embodiments, a prosthetic valve or a graft may be premounted on to the apparatus. With any of the above embodiments, the apparatus may be configured to be delivered percutaneously or through open surgery. With any of the embodiments herein, the devices may be attached by a variety of techniques including sutures, preattached sutures and needles, shape memory clips that will engage tissue, anchors, other fastener device, or any combination of the above. It should be understood that the present invention may be adapted for use on other valves throughout the body. Embodiments of the present invention may be used with stented, stentless, mechanical, or other valves. Some embodiments may be used in open surgery or for off-pump, minimally invasive techniques. It should be understood that any of the devices disclosed herein may be adapted for use with the graft. It should be understood that any of the devices disclosed herein may be adapted for use with a blood flow containment device that will allow for off-pump valve replacement.

The publications discussed or cited herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed. The present application is related to copending U.S. Provisional Application No. 60/551,992 fully incorporated herein by reference. All publications mentioned herein are incorporated herein by reference to disclose and describe the structures and/or methods in connection with which the publications are cited.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

Expected variations or differences in the results are contemplated in accordance with the objects and practices of the present invention. It is intended, therefore, that the invention be defined by the scope of the claims which follow and that such claims be interpreted as broadly as is reasonable.

1. A method of valve replacement, the method comprising:
providing a valve excisor;
providing an apparatus having a valve prosthesis and a valve leaflet support, said apparatus having a first configuration and a second configuration;
accessing the aortic root without placing the patient on a heart-lung machine;
advancing the apparatus in the first configuration where the valve leaflet support is advanced through a valve, wherein the support is positioned below a valve annulus;
expanding said apparatus into a second configuration so that said support will engage said valve;
moving said valve leaflet support and valve excisor together to remove leaflets of the valve;
advancing penetrating members on said apparatus into valve tissue, said penetrating members being secured in said valve tissue and a valve prosthesis, and act as fasteners to hold said valve prosthesis in position.

2. The method of claim 1 wherein accessing the aortic root without placing the patient on a heart-lung machine further comprises attaching a graft to the aorta.

3. The method of claim 1 wherein accessing the aortic root without placing the patient on a heart-lung machine further comprises:
attaching a first graft to the aorta;
clamping said first graft;
attaching a second graft to the first graft, wherein the second graft includes a hemostatic cap on a proximal end to prevent excessive blood loss and said apparatus contained in the second graft, said apparatus movable out of said graft and into the aorta.

4. The method of claim 2 wherein said graft is compressed in an accordian-like fashion when the apparatus is advanced.

5. The method of claim 1 wherein said apparatus further comprises using a ratchet to move the valve support to engage it against said valve leaflets.

6. The method of claim 3 wherein said apparatus further comprises using a ratchet to move the valve support to engage it against said valve leaflets, said ratchet extending outward from the hemostatic cap.

7. The method of claim 1 further comprising injecting the patient with medication to cause temporary asystole.

8. The method of claim 1 further comprising injecting the patient with adenosine phosphate.

9. The method of claim 1 further comprising using a pericardial tent on said apparatus to shield leaflets on the valve prosthesis from being damaged by the cutting element.

10. The method of claim 1 wherein the valve excisor is turned with simultaneous proximal counter traction to remove the valve leaflets.

11. The method of claim 1 wherein the valve leaflets are captured between the pericardial tent and the cutting element.

12. The method of claim 1 wherein the apparatus is delivered minimally invasively.

13. The method of claim 1 wherein attaching comprises delivering a shape memory clip that holds the valve prosthesis to the tissue.

14. A valve delivery device comprising:
a heart valve prosthesis support having a proximal portion and a distal portion;
a plurality of fasteners ejectably mounted on the support;
a heart valve prosthesis being releasably coupled to said distal portion of said heart valve prosthesis support;
said heart valve prosthesis and support being configured for delivery to the heart through an aortotomy formed in the patient's aorta;
a valve excisor; and
an anvil movable along a longitudinal axis of the device to engage tissue disposed between the anvil and the valve prosthesis.

15. The device of claim 14 further comprising a pericardial tent positioned to capture valve leaflets between the tent and the valve excisor.

16. A valve delivery device comprising:
a heart valve prosthesis support having a proximal portion and a distal portion;
a plurality of fasteners ejectably mounted on the support;
a heart valve prosthesis being releasably coupled to said distal portion of said heart valve prosthesis support;
said heart valve prosthesis and support being configured for delivery to the heart through an aortotomy formed in the patient's aorta;
a valve cutting element;
a pericardial tent positioned to capture valve leaflets between the tent and the valve cutting element; and
a leaflet support movable along a longitudinal axis of the device to engage tissue disposed between the support and the valve prosthesis.

17. An end-to-side access device comprising:
a first portion of a tissue clamp;
a second portion of the tissue clamp, said second portion having a collapsed configuration and an expanded configuration;
a tissue cutter;
a hollow shaft;
wherein the second portion may be delivered through said hollow shaft into the vessel to engage an inner surface of the wall of the vessel while the first portion engages an outer surface of the wall;
wherein the tissue cutter is configured to cut the tissue engaged by the tissue clamp, said first portion and second portion slidable to remove tissue engaged between the tissue clamp from the vessel.

18. The device of claim 17 further comprising a graft housing said tissue cutter and tissue clamp.

19. The device of claim 18 further comprising a ring of fasteners used to secure the graft to the tissue.

20. The device of claim 18 further comprising a hemostatic cap on the graft.

21. A device for use with a heart surgery device in an off-pump heart procedure, the device comprising:
a blood containment device wherein one portion of the device is configured to be attached to a blood vessel while blood is flowing in the vessel;
a hemostatic cap located on another portion of the blood containment device, wherein the hemostatic cap allows a portion of the heart device to extend through the hemostatic cap without creating a leak; and
a vent allowing for air to be removed from the containment device.

22. The device of claim 21 wherein the blood containment device is sized to house at least a portion of a valve prosthesis delivery device therein.

23. The device of claim 21 further comprising a release device that allows the portion of the device with the hemostatic cap to be removed from the portion of the containment device attached to the blood vessel.

24. The device of claim 21 wherein the blood containment device is a graft.

25. The device of claim 21 wherein the blood containment device is a graft made of a pliable, flexible material that can be clamped to created a fluid seal.

26. The device of claim 21 wherein the blood containment device is attached to the blood vessel without substantial blood leakage and without creating a life-threatening loss of blood.

27. The device of claim 21 wherein the blood containment device houses a portion of a multi-fire valve prosthesis delivery device.

28. The device of claim 21 wherein the blood containment device has a diameter of less then 30 mm.

29. The device of claim 21 wherein the blood containment device has a diameter of less then 20 mm.

30. The device of claim 21 wherein the blood containment device has a diameter of less then 10 mm.

31. The device of claim 21 wherein the blood containment device is sized to fit inside a four inch incision into the sternum.

32. A method of valve replacement, the method comprising:
providing a valve prosthesis delivery apparatus;
attaching a blood containment device to the aorta to prevent substantial loss of blood when the aorta is cut to provide access to an interior of the heart;
cutting the aorta to access the aortic root without placing the patient on a heart-lung machine;
advancing the apparatus to the area of the diseased valve;
delivering the valve to the target site;
retracing the valve prosthesis delivery apparatus into the blood containment device; and
sealing off a portion of the containment device to allow the delivery device contained therein to be removed without significant blood loss.

33. The method of claim 32 wherein the blood containment device is sized to house at least a portion of a valve prosthesis delivery device therein.

34. The method of claim 32 further comprising an release device that allows a portion of the device with a hemostatic cap to be removed from the portion of the containment device attached to the aorta.

35. The method of claim 32 wherein the blood containment device is a graft.

36. The method of claim 32 wherein the blood containment device is a graft made of a pliable, flexible material that can be clamped to created a fluid seal.

37. The method of claim 32 wherein the blood containment device is attached to the blood vessel without substantial blood leakage and without creating a life-threatening loss of blood.

38. The method of claim 32 wherein the blood containment device houses a portion of a multi-fire valve prosthesis delivery device.
